(19) United States
(12) Patent Application Publication
(10) Pub. No.: US 2026/0021121 A1
(43) Pub. Date: Jan. 22, 2026

(54) CANNABIS COMPOSITIONS AND METHODS

(71) Applicant: AIP Asset Management Inc., Toronto (CA)

(72) Inventors: Guy Chamberland, Orleans (CA); Charles Campbell, Kanata (CA); Randy Ringuette, Nepean (CA); Jennifer Dorothy Bassett, Orleans (CA); Ofer Yifrach-Stav, Cote Saint Luc (CA); Andrien Rackov, Orleans (CA); Peter Ford, Orleans (CA); Diane Botelho, Orleans (CA)

(73) Assignee: AIP Asset Management Inc., Toronto (CA)

(21) Appl. No.: 19/064,863

(22) Filed: Feb. 27, 2025

Related U.S. Application Data
(63) Continuation of application No. 17/415,258, now abandoned.

(30) Foreign Application Priority Data
Dec. 18, 2018 (CA) 3027876

(51) Int. Cl. A61K 31/00; A61K 9/00; A61K 9/20; A61K 36/185

(52) U.S. Cl. CPC A61K 31/658; A61K 9/007; A61K 9/2095; A61K 36/3482

(57) ABSTRACT

Provided are pharmaceutical compositions comprising cannabis such that when combusted produces cannabidiolic acid (CBDA) for inhalation. Also provided are pharmaceutical composition for inhalation by a subject comprising tetrahy drocannabinol (THC), tetrahydrocannabinolic acid (THCA), cannabidiol (CBD), cannabidiolic acid (CBDA), and cannabigerol (CBG). Also provided are methods of enhancing cannabidiolic acid (CBDA) in a pharmaceutical composition for inhalation. Methods of use of embodiments of the pharmaceutical composition and kits comprising embodiments of the pharmaceutical composition are provided herein. Unit dosage form medicaments and compositions for vaporization by a subject comprising cannabinoids are also disclosed herein.

### RELATED APPLICATIONS

This application is a continuation of and claims priority under 35 U.S.C. § 120 to U.S. patent application U.S. Ser. No. 17/415,258, filed Jun. 17, 2021, which is a national stage filing under 35 U.S.C. § 371 of International PCT Application, PCT/CA2019/051833, filed Dec. 17, 2019, which claims priority to Canadian Application No. 3,027,876, filed on Dec. 18, 2018, each of which is incorporated herein by reference.

### FIELD OF INVENTION

The present invention relates to cannabis compositions and related methods of preparation and use. More specifically, the present invention relates to inhalation of medicinal components derived from cannabis.

### BACKGROUND OF THE INVENTION

Cannabis is a genus of flowering plants in the family Cannabaceae and may be known by other names, such as marijuana, weed or pot. Cannabis is typically consumed by users as a recreational drug often by smoking dried plant material or an extract. Other dosage forms include oral consumption (such as an oil extract or alcoholic tincture) and topical administration. While recreational use is still prohibited in many countries, medical use of cannabis is gaining traction around the world. Unfortunately, the criminalization of cannabis has greatly slowed its development as a therapeutic despite growing evidence of its medicinal properties.

Cannabis plants produce molecules classified as cannabinoids or phytocannabinoids, some of which are psychoactive, such as tetrahydrocannabinol (THC), and some are considered non-psychoactive, such as tetrahydrocannabinolic acid (THCA or THCa), cannabidiol (CBD), cannabinol (CBN) and cannabigerol (CBG). Cannabis plants may also produce non-cannabinolic compounds, such as terpenes (examples include myrcene, limonene, linalool, caryophyllene, humulene and others). The cannabinoids may be produced by the plant in an “acid” form which converts to a “neutral” form by a process called decarboxylation.

Cannabinoid acids, such as THCA and CBDA, are found in variable quantities in fresh, undried cannabis, but may progressively decarboxylate to their neutral forms with drying, such as THC and CBD, respectively. Decarboxylation may occur while heating such as when cannabis is smoked or cooked into cannabis edibles. Vaporization or combustion of cannabis often results in the complete conversion of cannabinoid acids to cannabinoids. Development of the best-known cannabinoid acid, THCA, as a useful therapeutic has been hindered due to the gradual or rapid decarboxylation of THCA to THC. Studies have shown that cannabinoid acids may have useful biological effects, such as immunomodulation, anti-inflammatory and others. There exists a need in the art for convenient delivery of cannabinoid acids and other medicinal components of cannabis to a patient.

There is a need in the art for effective methods to deliver the acid forms of cannabinoids, such as THCA and CBDA. There is also a need in the art for methods and compositions that provide a consistent, predictable dose to a patient.

### SUMMARY OF THE INVENTION

According to an embodiment of the present invention, there is provided a pharmaceutical composition comprising cannabis such that when combusted produces a cannabinoid acid, such as tetrahydrocannabinol acid (THCA), for inhalation. In some embodiments, a pharmaceutical composition comprising cannabis is disclosed such that when combusted produces cannabidiolic acid (CBDA) for inhalation.

According to an embodiment of the present invention, there is provided a pharmaceutical composition for inhalation by a subject comprising: tetrahydrocannabinol (THC); tetrahydrocannabinol acid (THCA); cannabidiol (CBD); cannabidiolic acid (CBDA); and cannabigerol (CBG).

In a further embodiment, the pharmaceutical composition comprises cannabis plant, plant matter or extracts of cannabis plants. The cannabis described herein may comprise Wilbur, Tamaracouta and Great Bear cultivars.

The present invention also contemplates pharmaceutical compositions in which the THC and THCA are present in the composition at a combined concentration of 1-30%. The THC and THCA are present in the composition at a combined concentration of 8-10%, such as 9.5%. In some embodiments, CBD and CBDA are present in the pharmaceutical composition at a combined concentration of 1-20%, for example 1-4% or 2.5%. In some embodiments, the CBDA in the pharmaceutical composition is at a concentration of 1-20%, for example 1-4% or 2.5%. In some embodiments, the CBG is present in the composition at a concentration of 0.1-2%, for example 0.4%.

According to an embodiment of the present invention, there is provided a pharmaceutical composition for inhalation by a subject comprising one or more cannabinoid in an acid form.

In some embodiments of the present invention, the pharmaceutical composition is generated from smoke. In some cases, the smoke comprises particulate matter. In some embodiments, the pharmaceutical composition is generated from vapor. In these embodiments and others, the vapor comprises particular matter. In a further embodiment, the particulate matter of the smoke or vapor may comprise the acid form of the one or more cannabinoid. The one or more cannabinoid may be tetrahydrocannabinolic acid (THCA) or cannabidiolic acid (CBDA).

According to an embodiment of the present invention, there is provided a pellet comprising compressed plant matter, the plant matter comprising: cannabidiol (CBD); and cannabidiolic acid (CBDA), wherein a least a portion of the CBDA remains in the acid form during consumption.

According to an embodiment of the present invention, there is provided a pellet comprising compressed plant matter, the plant matter comprising: tetrahydrocannabinol (THC); and tetrahydrocannabinolic acid (THCA), wherein a least a portion of the THCA remains in the acid form during consumption.

In some embodiments of the present invention, the plant matter is cannabis. In some embodiments, the pellet comprises CBD and CBDA at a combined content of 2-3%. The pellet may comprise tetrahydrocannabinol (THC), tetrahydrocannabinolic acid (THCA), and cannabigerol (CBG). In a further embodiment, the THC and THCA combined content is 8-11% and the cannabigerol (CBG) content is 0.05-0.48%. The THC and THCA combined content may be 9.5%, the CBD and CBDA combined content may be 2.5% and CBG content may be 0.4%. In further embodiments, the plant matter comprises Wilbur, Tamaracouta and Great Bear cultivars of cannabis.

According to an embodiment of the present invention, there is provided a pellet comprising cannabis plant matter, the cannabis plant matter formed into a pellet shape by compression. The pellets described herein may be formed by a manual tablet compressor. In further embodiments, the plant matter is compressed at a pressure of 500-1500 psi. In some embodiments, the pressure is 1000-1250 psi.

According to an embodiment of the present invention, there is provided a method of enhancing cannabidiolic acid (CBDA) in a pharmaceutical composition for inhalation.

According to an embodiment of the present invention there is provided a method of use of a cannabis pellet comprising a pharmaceutical composition, the pharmaceutical composition comprising cannabinoids in an acid form. Embodiments of the method comprise inserting the cannabis pellet into a bowl of a smoking apparatus, the smoking apparatus comprising a body defining the bowl in fluid communication with a breathing passage, igniting and combusting the cannabis pellet within the bowl sufficient to generate smoke, and inhaling the smoke through the breathing passage. Further embodiments of the method comprises smoking apparatuses that are a pipe, vaporizer, bong, rolling papers, huka, and others.

According to an embodiment of the invention, a kit is provided for providing a pharmaceutical composition for inhalation. Embodiments of the kit comprise
a) a pellet comprising compressed cannabis plant matter and the pharmaceutical composition, the pellet comprising tetrahydrocannabinol acid (THCA), cannabidiolic acid (CBDA), or both;
b) an air tight container for containing the pharmaceutical composition, the air tight container comprising an inert fluid;
c) one or more sets of instructions for practicing the any of the methods described herein, using any of any embodiments described herein, interpreting the data obtained by practicing any of the methods described herein,
d) a smoking apparatus comprising a body defining a receptacle for receiving the pharmaceutical composition in fluid communication with a breathing passage; or
e) a combination of any of a)-d)

According to an embodiment of the current invention, unit dosage form medicaments for vaporization by a subject are disclosed. The medicament comprises a non-combustible substrate and a cannabinoid in a neutral form embedded on the non-combustible substrate.

In some embodiments, the cannabinoid is a synthetic cannabinoid. In further embodiments, the cannabinoid is THC. In further embodiments, the cannabinoid is CBD. In yet further embodiments, the cannabinoid is THC and CBD. In a further embodiment, the non-combustible substrate is a steel mesh. In some embodiments, the medicament is free of any additional excipient, oil or carrier. In one or more embodiments, the unit dosage form is a single use dosage. In further embodiments, the cannabinoid comprises 0.1-50 mg of THC. In yet a further embodiment, the cannabinoid comprises 13.9-18.9 mg of THC. In some embodiments, the cannabinoid comprises 0.1-50 mg of CBD. In further embodiments, the cannabinoid comprises 4.2-5.5 mg of CBD. In these embodiments and others, the medicament is heated to a temperature of 180-230° C., such as 210° C.

Uses of embodiments of the unit dosage form medicament are disclosed herein. In some embodiments the use is such that vaporization of the unit dosage form medicament results in a therapeutically effective dose of 6.6-8.1 mg of THC. In some embodiments, the use is such that vaporization of the unit dosage form medicament results in a therapeutically effective dose of 2.8-3.5 mg of CBD.

According to an embodiment of the present invention, vapor compositions comprising a therapeutically effective dose of 6.6-8.1 mg of THC. In these embodiments and others, the vapor composition further comprises a therapeutically effective dose of 4.2-5.5 mg of CBD.

According to an embodiment of the present invention, compositions for vaporization by a subject are disclosed herein. The composition comprises a non-combustible substrate and a cannabinoid in a neutral form embedded on the non-combustible substrate.

In some embodiments, the cannabinoid is a synthetic cannabinoid. In these embodiments and others, the cannabinoid is THC, CBD, or a combination thereof. In some embodiments, the non-combustible substrate is a steel mesh. In further embodiments, the composition does not comprise an oil, wax or carrier. The composition may be in unit dose form. The composition may be in a single unit dose form. In some embodiments, the cannabinoid comprises 0.1-50 mg of THC. In these embodiments and others, the cannabinoid comprises 13.9-18.9 mg of THC. In some embodiments, the cannabinoid comprises 0.1-50 mg of CBD. In these embodiments and others, the cannabinoid comprises 4.2-5.5 mg of CBD. In a further embodiment, the composition is heated to a temperature of 180-230° C. In some embodiments, the composition is heated to a temperature of 210° C.

Uses of embodiments of the composition are disclosed herein. In some embodiments the use is such that vaporization of the unit dosage form medicament results in a therapeutically effective dose of 6.6-8.1 mg of THC. In some embodiments, the use is such that vaporization of the unit dosage form medicament results in a therapeutically effective dose of 2.8-3.5 mg of CBD.

According to an embodiment of the present invention, methods of preparation of a unit dosage form medicament or composition, comprising a non-combustible substrate and a cannabinoid in a neutral form embedded on the non-combustible substrate, are disclosed herein. The method comprises: disbursing a liquid composition onto the non-combustible substrate, the liquid composition comprising the cannabinoid dissolved in a solvent, and drying the liquid composition to remove the solvent and embed the cannabinoid onto the non-combustible substrate.

In some embodiments of the methods disclosed herein, the cannabinoid is a synthetic cannabinoid. In further embodiments, the method further comprises the step of dissolving the cannabinoid in a solvent to form the liquid composition. In yet further embodiments, prior to dissolving, the method further comprises mixing of a secondary cannabinoid with the cannabinoid. In some embodiments, the cannabinoid is THC and the secondary cannabinoid is CBD. In yet further embodiments, drying comprises drying in a vacuum oven. In some embodiments, one or more steps of the method are conducted in an oxygen-free environment. In a further embodiment, the one or more steps are conducted under nitrogen gas. In some embodiments, the solvent is ethanol. In some embodiments, the cannabinoid is one or more of THC or CBD. In one or more embodiments, the cannabinoid is THC and CBD.

According to an embodiment of the present invention, kits for providing a pharmaceutical composition for inhalation, are disclosed herein. The kit comprises
a) the unit dosage form medicament of any embodiment described hereinabove,
b) an air tight container for containing the unit dosage form medicament, the air tight container comprising an inert fluid;
c) a vaporizing apparatus comprising a body defining a receptacle for receiving the pharmaceutical composition in fluid communication with a breathing passage; or
d) a combination of any one of a)-c).

This summary of the invention does not necessarily describe all features of the invention.

### BRIEF DESCRIPTION OF THE DRAWINGS

These and other features of the invention will become more apparent from the following description in which reference is made to the appended drawings wherein:

FIG. 1 shows a tablet that formed under low pressure after use by a user.

FIG. 2 depicts a flow diagram of an example of the manufacturing process.

FIG. 3 depicts a schematic of the production of example cannabinoids and transformation pathways.

FIG. 4 depicts a schematic of a pipe smoking setup used to collect smoke for analysis.

FIG. 5 depicts cannabis pellets used for smoke and vapor experiments.

FIG. 6A depicts Mainstream smoke passing through impinger collection system.

FIG. 6B depicts a side intake of a Raydiator pipe fitted and blocked with a Teflon™ plug.

FIG. 7 depicts ash remaining from a cannabis pellet that is considered to have burned very well.

FIG. 8 depicts a visual comparison of a smaller 30 mL impinger (left) to a larger 60 mL impinger (right).

FIG. 9 depicts smoke collection using a filter pad. A 37 mm high-efficiency glass fiber filter pad, loaded in a cassette, was placed in line with 4 impingers, each containing 25 mL methanol. Air flow through the system was verified with a flow meter placed before the filter pad.

FIG. 10 depicts a filter pad covered in cannabis smoke particulate matter. The white edges of the filter are representative of the filter color prior to smoke collection.

FIG. 11A-C depict a mighty vaporizer FIG. 11A shows a fully assembled vaporizer. FIG. 11B shows a heating chamber, where plant material is inserted, and can be accessed by removing a mouthpiece. FIG. 11C shows ground-up cannabis loaded into capsules that fit into the vaporizer heating chamber.

FIG. 12 depicts an observed effect of heating pellet material at 195° C. for six minutes in vaporizer.

FIG. 13 depicts snug fitting and loose fitting intact pellets in the Mighty vaporizer heating chamber.

FIG. 14 depicts examples of clean filter pads and filter pads containing trapped vapor.

FIG. 15 depicts a Raydiator pipe.

FIG. 16 is a time-course graph depicting absolute particulate mass collected on filter cassettes from intact PPP001 pellets heated in a Mighty vaporizer.

FIG. 17 is a visual comparison of PPP001 and PPP011. FIG. 17A shows the top and side view of a drip dosing capsule, which is composed of a stainless steel drip pad placed inside an aluminum dosing capsule. FIG. 17B shows the PPP001 cannabis plant pellet packaged inside a blister pack. FIGS. 17C and 17D, show the drip dosing capsule and the plant pellet fit into the filling chamber of the Mighty Medic®.

FIG. 17E-G depict how to access the receptacle of the Mighty Medic®.

FIG. 18 is a depiction of vapor collection system for profiling of volatile organic compounds.

FIG. 19 is an overlay of PPP001 (plant pellet; black) and PPP011 (drip dosing capsule; red) Vapor Chromatograms.

FIG. 20 is a schematic of the Vapor Collection System used in Example 8.

FIG. 21 is a graph depicting absolute recovery in vapor vs loading dose for PPP011 (THC).

FIG. 22 is a graph depicting absolute recovery in vapor vs loading dose for PPP011 (CBD).

FIG. 23 is a graph depicting relative recovery in vapor vs loading dose for PPP011 (THC).

FIG. 24 is a graph depicting relative recovery in vapor vs loading dose for PPP011 (CBD).

FIG. 25 is a depicting of the determination of a safe loading volume for PPP011.

FIG. 26 is a graph depicting absolute particle mass collected on filter cassettes using two puffing topographies to vape PPP011.

### DETAILED DESCRIPTION

The following description is of a preferred embodiment.

Described herein are pharmaceutical compositions comprising one or more cannabinoids in their respective acid forms, such as cannabidiolic acid (CBDA), and tetrahydrocannabinolic acid (THCA). Such compositions may be suitable for inhalation by vaporization or combustion. In some cases, the compositions comprise cannabis plant matter. In some cases, the compositions comprise cannabinoids from a synthesized source. The compositions may comprise an extract of a cannabis plant by a suitable process and dried. The composition may be isolates (such as pure phytocannabinoids), extracts, pure synthetic chemical entities, plant matter or a combination thereof. The composition may be an extract mixed with, placed on, or infused/embedded with a combustible non-plant substrate, such as cellulose and others. The composition may be infused/embedded on a non-combustible substrate, such as a steel mesh or wool. The composition may be for use in a pipe for combustion or placed within a dosing cassette for a vaporizer.

Pharmaceutical compositions, compositions and/or unit dose form medicaments disclosed herein may be formulated for delivery to a subject. A person of skill in the art will understand that a subject may be any suitable person or animal that may benefit from inhalation/delivery of the embodiments disclosed herein. In some cases, the subject is human.

The combustible non-plant substrate may also be a non-ionic surfactant and/or one polyol. At least one oil may be used, for example ethyl oleate, ethyl linoleate, caproic acid, caprylic acid, capric acid, or lauric acid, or a combination thereof. At least one oil may be a natural oil or be derived from a natural oil. The natural oil may be coconut oil, palm kernel oil, palm oil, lemon oil, or sunflower oil, or a combination thereof. At least one non-ionic surfactant may be a polyethoxylated castor oil, polyoxyethylene sorbitan monolaurate, polyoxyethylene sorbitan monoleate, tocopheryl polyethylene glycol succinate, and mixtures thereof.

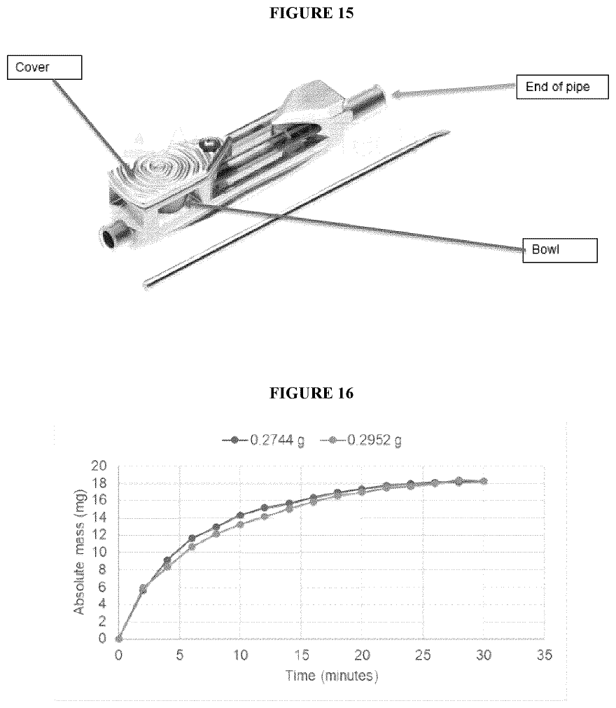

A pharmaceutical composition is disclosed that comprises cannabis such that when combusted produces a cannabinoid in its acidic form, such as tetrahydrocannabinol acid (THCA) or cannabidiolic acid (CBDA) for inhalation. The composition may be placed in a suitable apparatus, such as a pipe or vaporizer. The pipe may be made of a suitable material, such as glass or a metal (for example titanium or aluminum) or a combination. One example of a suitable pipe is the Raydiator pipe, see FIG. 15.

A pharmaceutical composition for inhalation may comprise a suitable cannabinoid in its acidic form, such as THCA or CBDA. Other components in the composition may include delta-9-tetrahydrocannabinol (A9-THC, THC), cannabidiol (CBD), CBN, CBC, CBL, CBE, CBT, CBGM, and cannabigerol (CBG), and their “-varin” homologues such as tetrahydrocannabivarin (THCV), cannabidivarin (CBDV), cannabichromevarin (CBCV), cannabigerivarin (CBGV), and cannabivarin (CBV). In some cases, the composition comprises cannabis, such as dried cannabis plant matter.

Cannabis plant matter may comprise various parts of the Cannabaceae plant, such as trichomes and flowers. Plant matter may include any part of the Cannabaceae plant that comprises any cannabinoids or non-cannabinoids. Cannabis plant matter may be a single cultivar or strain of the Cannabaceae plant or a combination of several strains of Cannabaceae. Such cultivars may come from the Cannabis Indica or Cannabis Sativa families. A person of skill in the art will understand that any cultivar of cannabis may be used that will provide compositions with the desired concentration of cannabinoids. For example, the cannabis plant matter may be a blend of Wilbur, Tamaracouta and Great Bear cultivars. Other possible cultivars include: Altair, Alyssa, Angie, Anka, CFX-1, CFX-2, CRS-1, CS, CanMa, Canda, Carmagnola, Carmen, Crag, Delores, Deni, ESTA-1, FINOLA, Fasamo, Fedrina 74, Felina 34, Ferimon, Fibranova, Fibriko, Fibrimon 24, Fibrimon 56, Georgina, GranMa, Grandi, Joey, Judy, Jutta, Katani, Kompolti, Kompolti Hibrid TC, Kompolti Sargaszaru, Laura Secord, Lovrin 110, Martha, Petera, Picolo, Quida, Silesia, UC-RGM, USO 14, USO 31, Uniko B, Victoria, X-59 (Hemp Nut), Yvonne, Zolotonosha 11, Zolotonosha 15, and others.

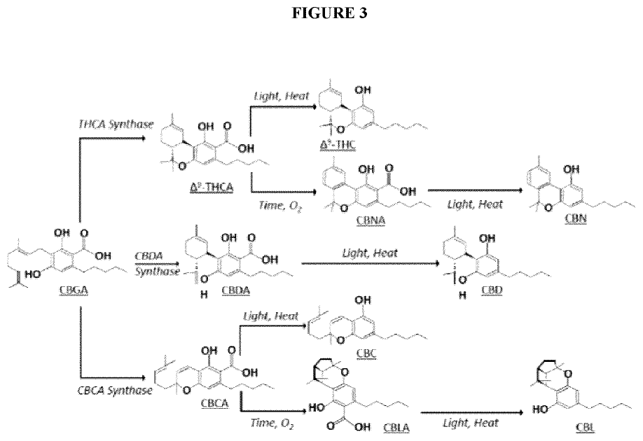

Referring to FIG. 3, cannabis plants may produce cannabinoids in an acidic or neutral form. Acidic, acid or charged form may refer to a cannabinoid prior to decarboxylation. The acid form of a cannabinoid may refer to a cannabinoid with at least one carboxylic acid moiety. The carboxylic acid moiety may result in the respective cannabinoid to have a negative charge at physiological pH. After decarboxylation, the cannabinoid loses a carboxylic acid moiety and the respective cannabinoid is then referred to as the neutral form. For example, FIG. 3 depicts the enzymatic production of THCA, cannabidiolic acid (CBDA), and cannabichromenic acid (CBCA) by the cannabis plant from a common precursor, cannabigerolic acid (CBGA). Not pictured is the production of additional cannabinoids in a neutral or acid form, such as cannabigerol (CBG). Cannabinoids in the acidic form may convert to the neutral form via non-enzymatic processes, such as light, heat, time or a combination. These non-enzymatic processes may be referred to as degradation.

Cannabinoids, such as THCA, may transform to an acidic form of another cannabinoid, such as cannabinol (CBD) via a non-enzymatic process. Cannabinoids may refer to any cannabinoid in the neutral or acidic form unless otherwise stated, for example THC (tetrahydrocannabinol), THCA (tetrahydrocannabinolic acid), CBD (cannabidiol), CBDA (cannabidiolic acid), CBN (cannabinol), CBG (cannabigerol), CBC (cannabichromene), CBL (cannabicyclol), CBV (cannabivarin), THCV (tetrahydrocannabivarin), CBDV (cannabidivarin), CBCV (cannabichromevarin), CBGV (cannabigerovarin), CBGM (cannabigerol monomethyl ether), CBE (cannabielsoin), CBT (cannabicitran) and others.

The pharmaceutical composition may have a suitable distribution of cannabinoids and non-cannabinoids in a neutral or acid form. The THC may be present in the composition at a concentration of 0.001-30%, or anywhere in that range for example 8-10% and 9.5%. THCA may present in the composition at a concentration of 1-20%, or anywhere in that range. CBD may be present in the composition at a concentration of 0.1-25% or anywhere in that range for example 2.5%. CBG may be present in the composition at a concentration of 0.01-5% or anywhere in that range, for example 0.4%. Cannabinoids in plant matter may have a lower cannabinoid content than an extract from plant matter. Extracts may be derived from plant matter or synthetic and may have concentrations of cannabinoids at near purity, such as 95% or higher. Pharmaceutical composition made of plant material may comprise cannabinoids with neutral forms below 10% of the total amount of cannabinoid in the neutral and acid forms. In some cases, degradation can alter the relative concentration of cannabinoids in the acidic or neutral form. Degradation may take the form of heat or light. Light or heat may convert the acidic form of a cannabinoid to its neutral form, see for example FIG. 3.

Suitable concentrations of cannabinoids may include any value or set of values within 0.001-100%, for example: 0.001, 0.002, 0.003, 0.004, 0.005, 0.006, 0.007, 0.008, 0.009, 0.01, 0.015, 0.02, 0.025, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99% and others.

Concentration or % may refer to w/w of the metabolite in the dried plant material or extract. Other concentrations than the concentrations disclosed are considered. Content and concentration may be used interchangeably. In some cases, a total or combined content may be disclosed. The total of combined content may refer to the total concentration of a cannabinoid in its neutral and acidic form, such as THC/THCA, and CBD/CBDA.

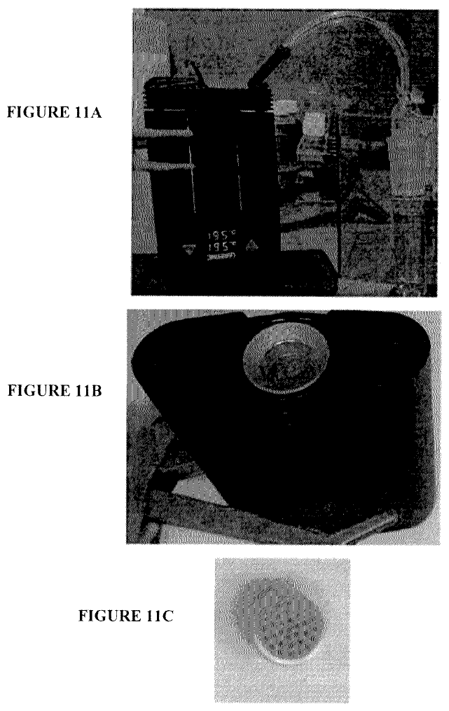

The pharmaceutical compositions, compositions and/or unit form medicaments, may be administered by inhalation. Inhalation or smoking of the pharmaceutical composition may occur by a suitable means, such as vaporization or combustion. Vaporization or combustion may be achieved with a smoking apparatus. For vaporization, a dry herb vaporizer such as the Mighty Vaporizer, may be used (FIGS. 11A-C). Combustion may be conducted using a smoking apparatus, for example a pipe such as a titanium Raydiator pipe. In use, the pharmaceutical composition may be inserted into the smoking or vaporizing apparatus. Subsequently, the user may heat or ignite the composition with a flame (from a lighter) or other heat source, and then begin to inhale the smoke or vapor. The patient may repeatedly inhale the smoke or vapor until the smoking apparatus no longer generates smoke or vapor. During combustion, the composition may be generated from smoke and particulate matter. If the composition is vaporized, the pharmaceutical composition may be delivered as particulate matter in the vapor.

Smoking or inhalation may include consumption of smoke derived from combustion, particulate matter or aerosols therein, and vapours through the respiratory system of a user. Aerosolization may be understood as the process or act of converting a physical substance into the form of particles small and light enough to be carried on the air. Any suitable aerosolization device may be used with the pharmaceutical composition, such as nebulizers, pressurized metered dose inhalers (MDIs), breath-activated inhalers (including dry powder inhalers), and other inhalation drug delivery systems. Consumption may be understood as the ingestion of the pharmaceutical composition.

The pharmaceutical compositions described herein may be contained in particulate matter generated by combustion or vaporization. The particulate matter may comprise acid forms of cannabinoids, such as THCA and CBDA. Inhalation of the particulate matter may result in pulmonary delivery of the active ingredients of the composition. Aerosolization of the composition by vaporization or combustion may result in small non-combusted (or non-vaporized) particulate matter being generated and carried with the smoke (or vapors) into the lungs of a user. The non-combusted material may contain the acid forms of the one or more cannabinoids. This may occur due to incomplete decarboxylation of the composition.

The pharmaceutical compositions disclosed herein may be compressed into a pellet. The pellet may comprise compressed plant matter, such as cannabis. In some cases, non-plant matter may be used. The plant matter may comprise cannabinoids in their acid form, such as THCA and CBDA. The plant matter may comprise cannabinoids in their neutral and acid forms, such as THC/THCA and CBD/CBDA. The compressed pellet may retain THCA during consumption by inhalation. The compression of the plant matter may prevent the decarboxylation of one or more cannabinoids during combustion or vaporization. Compression may assist in aerosolization of particulate plant matter and subsequent inhalation of cannabinoids in acid form by a user. In such cases, the compressed compositions may prevent or decrease the amount of combusted material per inhalation and increase the aerosolization of non-combusted material.

The content of cannabinoids in the pellet may be standardized. For example, the pellet may be prepared to achieve THC+THCA combined content of 9.5%, CBD and CBDA combined content of 2.5% and CBG content of 0.4%. One method to achieve standardization is to blend multiple strains/cultivars of cannabis. For example, Wilbur, Tamaracouta and Great Bear cultivars of cannabis may be blended to achieve desired concentrations of active ingredients.

The pellet may be manufactured by a suitable method. A flow diagram of an example of the manufacturing process is shown in FIG. 2. The active pharmaceutical ingredient, such as cannabis, may be first ground in a grinder to the desired consistency. In cases where a blend of cultivars is desired, the blending may occur before or after grinding. The plant matter may be processed such that consistent, uniformly sized particles are formed. This may assist with proper blending and formation of a homogenous blend.

After grinding and mixing, the plant matter is transferred to the pellet processing equipment. In some cases, a manual tablet compressor is used to compress the plant matter into a pellet. A range of pressures may be used for pellet compression, for example 500-1500 psi. In some cases, a tighter pellet and higher compression is needed, for example 1250 psi. A tighter, more compressed pellet may be more resistant to cracking or breaking during packaging or handling by a user. Tighter pellets may also generate more cannabinoids in the acidic form through increased aerosolization and particulate matter.

Pellet formation may occur in a temperature and humidity controlled manner. In some cases, the humidity of the environment surrounding the pellet compression apparatus, such as a manual tablet compressor, is controlled. In some cases, humidity is desirable to prevent drying of the ground plant matter. Drying of the plant matter may prevent proper clumping and pellet formation. Drying may also facilitate decarboxylation. Examples of a suitable range of humidity levels includes 5-30%, or any numbers within or outside the range for example 6-8%. Humidity may be within the range of 20-60%. Temperature during pellet formation may be controlled. High temperatures may generate resin or oil from the plant matter. In some cases, the resin or oil is undesirable due to poor pellet formation and increased decarboxylation. Temperature may be monitored by conversion of THCA or CBDA to THC and CBD, respectively. If more than 10% of the available acid forms converts, the pellet may be rejected. Suitable ranges of temperature during pellet formation includes 10-50° C. any number within that range, such as 10-25° C.

The plant matter may be compressed into a pellet shape. The pellet shape may resemble a flat disk. The pellet shape may have dimensions suitable to fit in the bowl of a vaporizer or combustion apparatus (such as a pipe, bong, and others). Other suitable shapes include pellets, discs, beads and others.

In an exemplary embodiment, a 280 mg dry compressed cannabis pellet is formed from a blend of three cultivars of cannabis (Wilbur, Tamaracouta and Great Bear). The cultivars are ground separately and combined into a homogenous blend to achieve a content of 9.5% THC and THCA combined, 2.5% CBD and CBDA combined, 0.4% CBG and various terpenes. The homogenous blend is placed into a manual tablet compressor and pressed into a flat disk pellet at 1000-1250 psi. The humidity in the facility during compression is controlled at 20-60% and the temperature at 15-25° C. If the pellet passes quality assurance, it is packed in an air free container, such as a blister pack. To prevent degradation, the pellet is stored in a vacuum sealed container, such as a blister pack, or under nitrogen. The pellet may be stored under nitrogen gas to prevent oxidation of any active pharmaceutical ingredients.

The pharmaceutical composition may be provided in a kit. The kit may comprise the pellet with the pharmaceutical composition. An air tight container for containing the pharmaceutical composition may be included. The air tight container may comprise an inert fluid, such as nitrogen gas. Instructions for use may be included. In some cases, a smoking apparatus comprising a body that defines a bowl for receiving the pharmaceutical composition in fluid communication with a breathing passage is included in the kit. The pellet may be shaped to fit within the bowl of the smoking apparatus, such as the Raydiator pipe (FIG. 15).

An exemplary method of use of the pellet is disclosed. A pellet comprising any pharmaceutical composition with one or more cannabinoids in an acid form may be inserted into a smoking apparatus. The smoking apparatus may comprise a body that defines a bowl in fluid communication with a breathing passage that terminates in an aperture. The pellet may be placed in the bowl of the smoking apparatus and ignited to combustion by a suitable means, such as a butane lighter. The pellet may be combusted sufficiently to generate smoke or vapor. The smoke or vapor may travel through the breathing passage into the lungs of a user. The combination of the grounded and compressed dried plant material in the pellet at a controlled compression pressure in the small space for combustion may lead to the delivery of the active pharmaceutical ingredients, including the acid forms of the cannabinols.

The smoking apparatus and pellet may act as a device that converts an inactive substance containing cannabinoids and terpenes into an aerosolized drug mixture of cannabinoids (in acidic and neutral forms) and terpenes to the patient. Delivery of a blend of cannabinolic acids and neutral substances with or without terpenes may create a drug substance for the treatment of pain, inflammation, psychiatric conditions, and others.

Described herein are unit dosage form medicaments and compositions comprising one or more cannabinoids on a non-combustible substrate. Unit dosage form medicaments will be understood by a person of skill in the art to refer to a discrete dosage form for a subject. Embodiments of the unit dosage form medicament may be for a single use by a subject. Unit dosage form medicaments, and other embodiments of the present invention, may offer several advantages, such as consistency of dose, control over dose, number of doses and scaling of doses for a subject. Embodiments of the unit dosage form medicaments may prevent repeat uses of the medicament. For example, embodiments comprising synthetic cannabinoids embedded on a non-combustible substrate will be suitable for a single use, because the cannabinoids may be substantially consumed after a single use in a medical device, such as a vaporizer. Such unit dosage form medicaments may also decrease the opportunity for a subject to abuse the medicament. Unit dosage form medicaments may decrease the potential for accidental overdose. The unit dosage form medicaments may also decrease degradation when packaged in single dose form. Degradation of dose may occur when multiple doses are in same container due to exposure to air and light after opening for prolonged periods.

Compositions and unit dosage form medicaments comprising non-plant derived cannabinoids are disclosed herein. A person of skill in the art will understand that synthetic cannabinoids refer to cannabinoids or cannabinoid-like entities that are from a synthetic source. The synthetic cannabinoids used in the unit dosage forms and compositions described herein, may be in the neutral form (for example THC and CBD) instead of the acid forms (for example THCA or CBDA). Such synthetic cannabinoids may be synthesized using good manufacturing practice (GMP) or other suitable protocols. In some cases, synthetic cannabinoids are considered semi-synthetic, such as when the starting material is isolated from a natural source. For example, the starting material may be the acid form (such as THCA) of the desired cannabinoid (such as THC). In other cases, synthetic cannabinoids are synthesized from pure starting materials. Using synthetically derived cannabinoids may offer several advantages. For example, cannabis plants may be contaminated with bacteria, fungi and other contaminants, which may negatively affect an immunocompromised individual when consuming the plant. Further, isolates from a plant extract comprising cannabinoids may be contaminated with small molecule contaminants, such as mycotoxins.

Methods for synthesizing neutral form cannabinoids, such as THC and CBD are known in the art. For example, suitable synthetic methods may be found in: CA Patent 3048298, US Patent application no. 2010/0210860, CA Patent 2469490, US Patent application no. 2017/0008868, U.S. Pat. No. 8,106,244, Stern, E.; Lambert, D. M.; Medicinal Chemistry Endeavors around the Phytocannabinoids, Chemistry & Biodiversity (2007), 4, 1707-1728, U.S. Pat. Nos. 7,186,850, 7,323,576, US Patent application no. 2015/0238428, Shah, V. J., Synthesis of cannabidiol stereoisomers and analogs as potential anticonvulsant agents. The University of Arizona, US Patent application no. 2012/0121621, World Health Organization 39^{th} ECDD (2017) Agenda item 5.1 on Cannabidiol (CBD), and the World Health Organization Expert Committee on Drug Dependence: Critical Review on Delta-9-tetrahydrocannbinol (2018), each of which are incorporated by reference in their entirety.

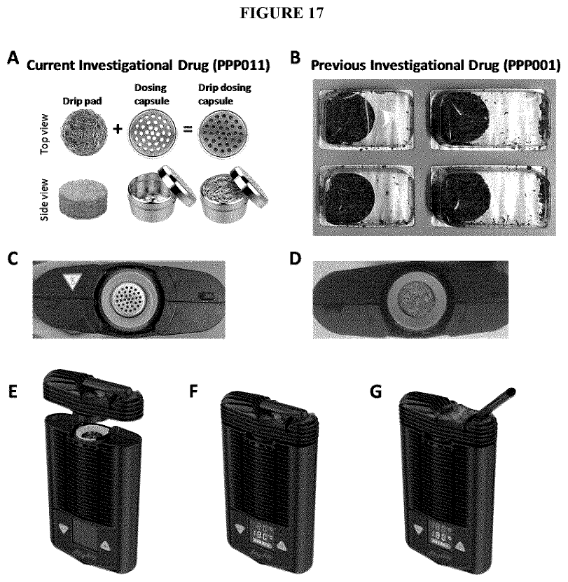

Disclosed herein are embodiments of the compositions and/or unit dosage form medicaments comprising cannabinoids and a non-combustible substrate for vaporization. A person of skill in the art will understand that a non-combustible substrate refers to a structure for embedding or infusing with the pharmaceutical composition (such as cannabinoids) that does not ignite or substantially degrade at vaporization or combustion temperatures. The non-combustible substrate may be placed in a housing before loading into a receptacle of a smoking or vaporizing apparatus. For example, a non-combustible substrate, such as a stainless steel mesh, may be placed in a dosing capsule before loading into a vaporizer, such as the Mighty Medic® vaporizer. The dosing capsule may have a suitable shape, defining apertures for vapor to pass, and an example of such is pictured in FIG. 17A. The dosing capsule may be any dosing capsule known in the art, such as those sold by Storz & Bickel.

The non-combustible substrate may be able to transmit heat and/or hot air for embedding the compositions. The substrate may have a suitable structure, such as a mesh that defines pores for embedding or infusing the compositions. Embedding or infusing will be understood by a person of skill in the art to refer to the interaction of the substrate and the compositions on the surface of the substrate and/or within the substrate, such as in the pores. Examples of suitable non-combustible substrates include substrates made of pure or alloy metal, such as steel mesh, aluminum mesh, stainless steel mesh (for example stainless steel 304 mesh), and others. Impregnated material may be used, for example bio-ceramics. Examples of suitable substrates are known in the art, for example: Dhand, R. Nebulizers that use a vibrating mesh or plate with multiple apertures to generate aerosol. Respiratory Care, 30 Nov. 2002, 47(12):1406-16; Dolovich, MB; Dhand, R. Aerosol drug delivery: developments in device design and clinical use. Lancet, 2011, 377(9770):1032-45, each of which are herein incorporated by reference.

Embodiments of the composition or unit dosage forms for vaporization as described herein may be free of any additional excipient, oil or carrier. A person of skill in the art will understand that “any additional excipient, oil or carrier” would refer to excipients, oils or carriers that are known in the art, and would not refer to the combustible/non-combustible substrate or active ingredients of the composition, such as the cannabinoids, which may themselves be an oil. A person of skill in the art will also understand that trace amounts of oil, carriers, such as solvent, would not be considered an “additional excipient, oil or carrier”.

Embodiments of the composition or unit dosage forms described herein may be vaporized in use. The cannabinoids may be dispersed onto a non-combustible substrate (such as a drip pad) and placed inside a dosing capsule, such as those shown in FIG. 17. The vaporizer may provide heat to the composition to vaporize the contents of the drip pad. Suitable temperatures may be used, such as 175-240° C., 180° C.-230° C., and in some preferred embodiments, 210° C. After the composition is heated to desired vaporization temperatures, the composition may be consumed (or nearly consumed) in a single dose. Re-use of the drip pad after the first vaporization for a second use may not provide any cannabinoids to the subject.

Suitable temperatures for heating the compositions described herein may be used, for example 175-250° C., or any value or set of values within that range. For example, temperatures may include 175, 175.5, 176, 176.5, 177, 177.5, 178, 178.5, 179, 179.5, 180, 180.5, 181, 181.5, 182, 182.5, 183, 183.5, 184, 184.5, 185, 185.5, 186, 186.5, 187, 187.5, 188, 188.5, 189, 189.5, 190, 190.5, 191, 191.5, 192, 192.5, 193, 193.5, 194, 194.5, 195, 195.5, 196, 196.5, 197, 197.5, 198, 198.5, 199, 199.5, 200, 200.5, 201, 201.5, 202, 202.5, 203, 203.5, 204, 204.5, 205, 205.5, 206, 206.5, 207, 207.5, 208, 208.5, 209, 209.5, 210, 210.5, 211, 211.5, 212, 212.5, 213, 213.5, 214, 214.5, 215, 215.5, 216, 216.5, 217, 217.5, 218, 218.5, 219, 219.5, 220, 220.5, 221, 221.5, 222, 222.5, 223, 223.5, 224, 224.5, 225, 225.5, 226, 226.5, 227, 227.5, 228, 228.5, 229, 229.5, 230, 230.5, 231, 231.5, 232, 232.5, 233, 233.5, 234, 234.5, 235, 235.5, 236, 236.5, 237, 237.5, 238, 238.5, 239, 239.5, 240, 240.5, 241, 241.5, 242, 242.5, 243, 243.5, 244, 244.5, 245, 245.5, 246, 246.5, 247, 247.5, 248, 248.5, 249, 249.5° C. and others.

In certain embodiments, a pharmaceutically acceptable carrier, diluent, or excipient is contemplated. Such carrier, diluent or excipient may include any suitable carrier, diluent, or excipient known to the person of skill in the art having regard to the teachings herein. Examples of pharmaceutically acceptable excipients may include, but are not limited to, cellulose derivatives, sucrose, and starch. The person of skill in the art will recognize that pharmaceutically acceptable excipients may include suitable fillers, binders, lubricants, buffers, glidants, dispersants, and/or disentegrants known in the art (see, for example, Remington: The Science and Practice of Pharmacy (2006)). Examples of pharmaceutically acceptable carriers, diluents, and excipients may be found in, for example, Remington's Pharmaceutical Sciences (2000-20th edition) and in the United States Pharmacopeia: The National Formulary (USP 24 NF19) published in 1999. The skilled person having regard to the teachings herein will be aware of suitable pharmaceutically acceptable carriers, diluents, and excipients appropriate for formulating polypeptides and/or nucleic acids as described herein to the lung of a subject in need thereof. In certain embodiments, the polypeptides and/or nucleic acids and/or compositions as described herein may be formulated with a propellant or carrier gas, which may or may not be pressurized, as a pharmaceutically acceptable carrier.

Other delivery methods are contemplated, such as those known in the art. The person of skill in the art having regard to the teachings herein will be aware of a variety of suitable approaches and techniques for formulating agents for throat or nasal administration to the lung of a subject (i.e. for pulmonary delivery). Delivery of agents to the lung has been the subject of significant study, as described in, for example, Bodier-Montagutelli, E., et al., 2018, Designing inhaled protein therapeutics for topical lung delivery: what are the next steps?, Expert Opinion on Drug Delivery, 15(8): 729-736; Labiris, N. R., et al., 2003, Pulmonary Drug Delivery. Part II: The role of inhalant delivery devices and drug formulations in therapeutic effectiveness of aerosolized medications, Br J Clin Pharmacol, 56:600-612; and Ibrahim, M., et al., 2015, Inhalation drug delivery devices: technology update, Med Devices (Auckl), 8:131-9, each of which are herein incorporated by reference in their entirety. In certain embodiments, compositions and/or unit dose form medicaments as described herein may be for delivery to the lung using a suitable drug delivery device. In certain embodiments, drug delivery devices may take the form of pulmonary devices such as, inhalers, nebulizers, aerosols, puffers, nasal sprays, or other suitable delivery device for administration to the lung. Examples of lung delivery devices are described in, for example, U.S. Pat. Nos. 5,983,893, 6,732,732, US20070295332, U.S. Pat. Nos. 5,007,419, 4,832,015, US20040244794, US20100065048, US20030235555, US20050201951, and US20090000615, each of which are herein incorporated by reference in their entireties. Liposomal cannabinoid delivery systems may be used, for example those described in EP1109533, which is incorporated herein by reference. Examples of suitable drug delivery devices are known in the art, for example: Dhand, R. Nebulizers that use a vibrating mesh or plate with multiple apertures to generate aerosol. Respiratory Care, 30 Nov. 2002, 47(12):1406-16; Dolovich, MB; Dhand, R. Aerosol drug delivery: developments in device design and clinical use. Lancet, 2011, 377(9770):1032-45, each of which are herein incorporated by reference.

Compositions and unit dosage form medicaments disclosed herein may have a suitable dose. Cannabinoid concentrations described herein may be mass of cannabinoids loaded onto the substrate before vaporization. Some embodiments may be described as the effective dose of cannabinoids, which refers to the mass of the cannabinoids in the vapor delivered to the subject. Compositions and unit dosage form medicaments may comprise 0.01-50 mg of THC and/or 0.01-50 mg of CBD embedded into the non-combustible substrate. In some cases, the substrate has 13.9-18.9 mg of THC and/or 4.2-5.5 mg of CBD embedded into its structure. When the composition is vaporized, the unit dosage form medicament or composition may result in a therapeutically effective dose of 6.6-8.1 mg of THC and/or 2.8-3.5 mg of CBD. Other suitable cannabinoids are contemplated, such as THCA, CBDA, CBG and others.

Suitable concentrations of cannabinoids, such as THC and CBD, may be used in the compositions herein, for example 0.01-100 mg, or any value or set of values within that range. For example, concentrations may include 0.01, 0.02, 0.03, 0.04, 0.05, 0.06, 0.07, 0.08, 0.09, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 mg and others.

Disclosed herein are vapor compositions comprising a therapeutically effective dose of cannabinoids. A vapor composition will be understood by a person of skill in the art to be the aerosolized/vaporized cannabinoids from the compositions described herein. In some cases, the vapor composition comprises 0.01-100 mg of THC and/or 0.01-100 mg of CBD. In further embodiments, the vapor composition comprises 6.6-8.1 mg of THC and/or 4.2-5.5 mg of CBD. Other suitable cannabinoids are contemplated, such as THCA, CBDA, CBG and others.

Suitable concentrations of cannabinoids, such as THC and CBD, may be used in the vapor compositions herein, for example 0.01-100 mg, or any value or set of values within that range. For example, concentrations may include 0.01, 0.02, 0.03, 0.04, 0.05, 0.06, 0.07, 0.08, 0.09, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1, 1.5, 2, 2.5, 3, 3.5, 4, 4.5, 5, 5.5, 6, 6.5, 7, 7.5, 8, 8.5, 9, 9.5, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 mg and others.

A suitable ratio of THC:CBD may be used in any of the unit dosage form medicaments and/or compositions described herein. In compositions where a euphoric effect is desired, the ratio of THC:CBD may be greater than 1, for example 2:1, 3:1, 4:1 (THC:CBD) and others. In some embodiments, an anti-inflammatory or non-euphoric effect is desired. Such embodiments may have a ratio of THC:CBD that is less than 1, for example 1:2, 1:3, 1:4 (THC:CBD) and others.

The compositions and/or unit dosage form medicaments may be formulated as a single use dosage. In such cases, the dosage of the formulations are intended for a single delivery to one subject. In some embodiments, cannabinoids are embedded on the non-combustible substrate and heated in a vaporizer at 210° C. for a length of time, for example 0.1-10 minutes, or 2 minutes. Heating in a vaporizer may result in vaporization of the entire concentration of cannabinoids on the substrate, and delivery of the therapeutic dose in the resulting vapor. The composition or medicament may then be consumed, and subsequent heating may not substantially produce any vaporized cannabinoids. Single use dosages may be packaged such that each dosage is individually contained in an air-tight container with an inert fluid, such as nitrogen. Examples of suitable packaging are detailed herein.

Methods of preparation of unit dosage form medicaments and/or compositions comprising acid and neutral cannabinoids embedded on substrates are described herein. Preparing the unit dosage form medicament and/or compositions comprises disbursing a liquid composition onto the non-combustible substrate and drying the liquid composition to remove the solvent and embed the cannabinoid onto the non-combustible substrate. In embodiments, where the cannabinoid is a synthetic neutral or acid form cannabinoid, the preparation may be conducted in an oxygen-free environment with an inert fluid, such as nitrogen gas. Reducing the amount of oxygen that interacts with the synthetic cannabinoid may decrease the amount of degradation of the synthetic cannabinoid. Such embodiments of the method may comprise conducting any one of the steps described herein under nitrogen gas or vacuum. For example, drying the liquid composition to remove the solvent may be achieved in a vacuum oven. The liquid composition may be formed by dissolving the one or more cannabinoid in a suitable solvent, such as ethanol, and transferred to the substrate.

An exemplary embodiment of a method of preparation of unit dosage form medicaments and/or compositions comprising synthesized neutral cannabinoids is described herein. The steps described herein are conducted in an atmosphere of nitrogen gas unless described otherwise. First, a solution of THC in a suitable solvent, such as ethanol or THC/heptanes is dried to give an oil or film of pure THC. Another cannabinoid in a solid or oil form, such as CBD, is then mixed with the pure THC to a desired concentration. The mixture of pure cannabinoids is then dissolved in a solvent, such as ethanol, to give the liquid composition. The liquid composition is disbursed onto the non-combustible substrate, such as steel mesh, and dried in a vacuum oven. The oven is backfilled with nitrogen gas once the drying is complete.

The resulting unit dosage form medicaments and/or compositions are then packaged in a suitable oxygen-free container with an inert gas, for example mylar bags, blister paks or a combination thereof. Other primary packaging, secondary packaging, scavengers or sensors as described herein may be used.

Kits for providing a pharmaceutical composition or unit dose form medicament for inhalation are disclosed herein. Contemplated kits may comprise:
a) the unit dosage form medicaments or compositions as described herein;
b) an air tight container for containing the unit dosage form medicament, the air tight container comprising an inert fluid;
c) a vaporizing apparatus comprising a body defining a receptacle for receiving the pharmaceutical composition in fluid communication with a breathing passage; or
d) a combination of any one of a)-c).

The air tight container may comprise one or more scavenging devices within the container. For example, an oxygen scavenging and/or humidity scavenging (desiccant) may be inserted into the container. The scavenging devices may act to increase the shelf-life of the labile contents of the container. The devices may be disposed of after the composition or drug product is removed together with packaging. The air tight container may comprise a sensor to detect the presence of moisture and/or oxygen. In some cases, the sensors and scavenging devices are integrated together and/or with the packaging. Such devices and sensors are known in the art, for example those produced by Harro Höfliger and Siebler-Romaco. Other examples are known in the art, such as: https://www.packagingdigest.com/desiccants/pmp-Dialing%20in%20stable%20packaging%20for%20sensitive%20drugs-100701

The present invention will be further illustrated in the following examples.

### Examples

### Example 1: Pellet Design and Formation

Each lot is transferred into the grinder and is ground until the desired consistency is achieved and material passes through a 6 mm sieve. There are no excipients used to make the pellet. The pellet is made by simply compressing the drug substance (dried ground blend of the 3 cultivars).

The investigational new drug PPP001, is a pellet that may contain 280 mg dry compressed cannabis standardized to 9.5% total/combined delta-9-tetrahydrocannibinol (delta-9-THC or THC) and tetrahydrocannabinolic acid (THCA), 2.5% cannabidiol (CBD), 0.4% Cannabigerol (CBG), and several terpenes (myrcene, limonene, linalool, caryophyllene, humulene). Total or combined THC and THCA % may be equal to THCA+THC, where the great majority of the content may be the naturally occurring form of THCA, In addition, the formulation may contain 1 other cannabinoid at a measurable dose and terpenes. The efficacy of the PPP001 product is associated to the combination of these ingredients.

The drug substance, or Active Pharmaceutical Ingredient (API), may consist of 3 cultivars of Cannabis in a fixed ratio to obtain the target potency of 9.5% THC, 2.5% CBD and 0.4% CBG. The 3 cultivars are: Wilbur, Tamaracouta, Great Bear.

A single PPP001 drug pellet is inserted into the PPP-titanium pipe. Subsequently, the patient ignites the PPP001 pellet with a flame (from a lighter) and then begins to inhale the smoke. The patient repeatedly inhales the smoke until the titanium pipe no longer generates smoke.

A blend of 3 cultivars of Cannabis designed to obtain the target amount of 9.5% delta-9-THC and 2.5% CBD (Table 1).

The pellet dosage form was selected for several reasons: 1. Help control the burn rate of the dried cannabis thereby controlling the amount of cannabinoids absorbed per inspiration. 2. Help reduce second hand smoke since the compressed pellet does not continue to burn when the subject stops inhaling. 3. Use of a fixed dose approach to standardize dosing and systemic exposure.

Various compression pressures (500, 750, 1000 and 1250 psi) were tested to achieve the desired resistance to finger manipulation by the patient and to allow combustion. The laboratory designed an in-process control that assesses the burn time and determines mathematically the amount of THC consumed per puff. An artificial lung or breathing apparatus that simulates a breath of 250 cc at 3 mmHg was developed. After 8 puffs or inhalations, only 160 mg of PPP001 pellet remained. After 15 inhalations there is virtually no residue of plant material left. This provides approximately 20 mg of plant material for each inhalation. Upon ignition the pellet burned from the outside in, and from the inside out, resulting in little or no plant material left following 15 inhalations.

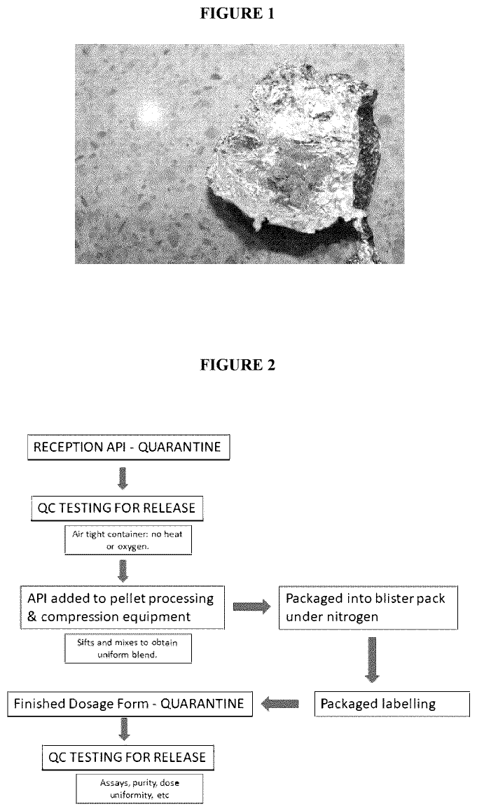

All ignition and inhalation were best performed at 1,000-1250 psi. The pellet itself was also robust, and less likely to break apart. FIG. 1 shows what happens to the lower (for example, 500 psi) pressure pellets when you try to pick up the pellet with fingers.

An assessment of the tablet-caplet making equipment revealed that these commercial systems generate too much heat for the compression of dried cannabis. The heat generated by the flow of the ground material and temperature at the surface of the compression piston causes the formation of a cannabis oil. Based on this information, Tetra BioPharma contracted the development of a pellet making system, based on the tablet-caplet production concept.

TABLE 1

| Component and Quality | | Strength (label claim) | |
|---|---|---|---|
| Standard (and Grade, if | | 9.5% THC, 2.5% CBD, 0.4% CBG with terpenes* | |
| applicable) | Function | Quantity per unit | % |
| Blend of 3 strains | Medicinal | 280 mg | 100% |
| | ingredient | | |
| Total | | 280 mg | 100% |

*myrcene, limonene, linalool, caryophyllene, humulene.

### Example 2: Description of Manufacturing Process and Process Controls (PPP001, Pellet)

FIG. 2 depicts a Flow diagram of the manufacturing process.

Detailed narrative description of the manufacturing process, including equipment type and working capacity, process parameters. A 280-mg pellet is made in a facility dedicated for processing and manufacturing of plant-based products. Qualified raw material is first loaded into a large-scale grinder, where it is processed into a uniform particle size and passed through a standardized mesh sieve. Moisture content of the ground mix is verified with a moisture analyzer and, if necessary, dried until it meets the predetermined specification for moisture content. The material is then mixed thoroughly using a blender, and the resulting API intermediate is packaged in quarantine pending QC testing and release. Released intermediate material is then loaded into a manual tablet press in a chamber designed to hold the quantity of the blend required to produce a 280-mg pellet. The material is then compressed into a pellet by direct compression and analyzed and verified for weight uniformity. The pellets are then packaged in blister packages and stored.

### Controls of Steps and Intermediates (PPP001, Pellet)

Summary of controls performed at the steps of the manufacturing process and on isolated intermediates: the manufacturing process of the PPP001 cannabis pellet is divided in two process sections: intermediate manufacturing process, and pellet manufacturing process. All raw materials and finished goods are qualified and released if conforming to pre-determined specifications to mitigate their direct impact on the critical quality attributes.

### Intermediate Manufacturing:

a. Step 1: Grinding
   i. Process control: verify particle size via sieve shaker with standardized-mesh sieves.
   ii. Process control: verify particle size via pellet compacity and friability via friability to manipulation; and via ignitability/combustibility via ignition/combustion tests.
b. Step 2: Drying
   i. Process control: verify humidity via loss on drying.
   ii. Process control: analysis of uniformity of pellet weights.
c. Step 3: Mixing of intermediate
   i. Process control: assay and uniformity of API (of samples taken from top, middle, and bottom) via assay.

### Pellet Manufacturing:

a. Step 1: Average die filling mass
   i. Process control: analysis of uniformity of pellet weights.
b. Step 2: Compression of pellet
   i. Process control: friability, ignition, combustion.

Manufacturing facility environmental conditions: Humidity: 35-65%; Temperature: 15-25° C.

### Humidity:

This step involves ensuring the correct humidity in the product used for the manufacturing (that the blend of ground dried cannabis is at the target humidity of 4-15%) to allow compression. This is more important for the placebo than the PPP001 drug.

Poor humidity control is detected in the intermediate via analysis of loss on drying, and in the finished product with the weight of the pellet. Inadequate control leads to rejection based on the 280-mg weight specification.

TABLE 2

Average weight: specification = Target
280 mg ±10% (range: 252-308 mg)
Uniformity - Weight variation (20 tablets):
specification = No pellets > ±10%

### Heat:

Heat should be avoided during the flow of the ground material and its compression. This aspect of the tablet/pellet making process is to ensure good dose uniformity. Heat generated by the compression process leads to the formation of a cannabis oil on the surface of the pellet and conversion from THCA/CBDA into THC/CBD. Inadequate control leads to rejection based on the change to THC relative to the amount analyzed specification.

The API and pellets must be stored in an air tight container (e.g., blister pack) and kept between 15-25 C to avoid degradation. Heat and or oxygen will convert the THCA into THC and this is readily detectable at finished product release testing step.

TABLE 3
Degradation: Assay/Quantification of D9-THC + D9-THC-acid: specification = Change in THCA to THC greater than 10%.
Degradation: Assay/Quantification of total CBD (CBD + CBDA): specification = Change in CBDA to CBD greater than 10%.

### Blending:

This step involves ensuring a homogenous blend in the product used for the manufacturing prior to compression. This step is also important for the placebo.

The dose uniformity studies show improvement subsequent to optimization of the blending process. The dose uniformity for the Phase 1 clinical batch had a % variability of 12.82% and 15.07% for THC and THCA, respectively. After optimizing the process, the dose uniformity for clinical batches was 5.59% and 5.13% for THC and THCA, respectively.

Inadequate control leads to rejection based on the dose uniformity specification. In fact, a stability batch was rejected post production based on this specification.

TABLE 4
Uniformity THC variation (20 pellets): specification = No pellet > ±10%
Uniformity THCa variation (20 pellets): specification = No pellet > ±10%
Uniformity CBD variation (20 pellets):
specification = NMT 5 pellets > ±10%
Uniformity CBDA variation (20 pellets):
specification = NMT 5 pellets > ±10%

### Example 3: Stability Data

A schematic depicting the production of exemplary cannabinoic acids and subsequent transformation to the related neutral form by conditions such as oxidation, heat and time is shown in FIG. 3. Table 5 shows 9-month stability data of a dried cannabis drug stored in a vault (not under nitrogen).

TABLE 5

| Analyte | Nov. 17, 2016 | Aug. 10, 2017 | % Change from Day 0 |
|---|---|---|---|
| D9-THC | 0.21 | 1.75 | ↑ 733% |
| D9-THCA | 9.68 | 6.02 | ↓ 37.8% |
| CBD | <0.05* | 0.68 | ↑ 1260% |
| CBDA | 3.07 | 2.03 | ↓ 33.9% |
| CBG | <0.05* | 0.12 | ↑ 140% |
| CBGA | 0.37 | 0.26 | ↓ 29.7% |
| Total D9-THC | 8.71 | 7.03 | ↓ 19.3% |
| Total CBD | 2.69 | 2.46 | ↓ 8.6% |
| Total CBG | 0.32 | 0.35 | ↑ 9.4% |

*assumed to be 0.05 for calculation.

### Example 4: Development of a Method for the Detection of Cannabinoids and Terpenes in Cannabis Smoke

Trapping Smoke from Cannabis Burned in a Pipe

A method was developed to capture smoke from cannabis pellets (PPP001) burned in a pipe (Raydiator Pipe). The method involved the use of impingers filled with applicable solvents for cannabinoid and terpene extraction. After pellets had been combusted and the smoke bubbled through solvent in the impingers, the solvent was analyzed for cannabinoids and terpenes using validated in-house analytical platforms. By extracting analytes from the smoke into a solution phase, the same methods used to analyze unburnt cannabis pellets could be applied. Cannabinoids were analyzed by high performance liquid chromatography with photodiode array detector (HPLC-DAD) and terpenes were analyzed by gas chromatography-mass spectrometry (GC-MS). The suite of cannabinoids and terpenes analyzed are given in Tables 1 and 2 respectively.

TABLE 6
Cannabinoids analyzed by HPLC
Cannabinoids

Delta 9-Tetrahydrocannabinol (Delta 9-THC)
Delta 9-Tetrahydrocannabinolic Acid (Delta 9-THCA)
Cannabidiol (CBD)
Cannabidiolic Acid (CBDA)
Cannabigerol (CBG)
Cannabigerolic Acid (CBGA)
Cannabinol (CBN)
Cannabichromene (CBC)
The reporting limit for cannabinoids is 0.001 mg/mL

TABLE 7
Terpenes analyzed by GC-MS
Terpenes

| | |
|---|---|
| Alpha pinene | Eucalyptol |
| Beta pinene | Trans-ocimene |
| Myrcene | Fenchol |
| Limonene | Borneol |
| Terpinolene | Valencene |
| Linalool | Cis-nerolidol |
| Terpineol | Trans-nerolidol |
| Caryophyllene | Guaiol |
| Humulene | Alpha-bisabolol |
| 3-carene | Sabinene |
| Cis-ocimene | |

The reporting limit for terpenes is 0.001 mg/mL

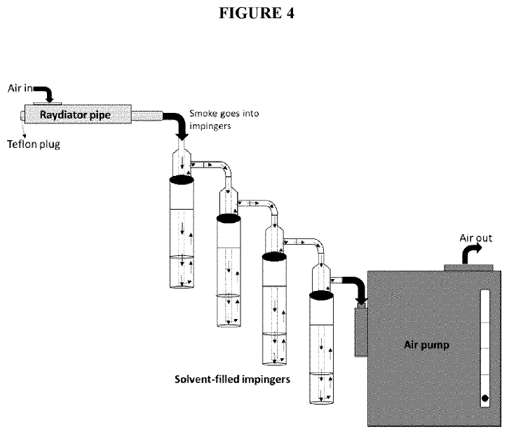

In order to trap cannabinoids and terpenes from smoke produced from the Raydiator pipe, the mouthpiece of the pipe was interfaced to several impingers which were connected in series and loaded with the suitable extraction solvent. A pump connected to the last impinger was used to draw smoke through the impinger setup. A schematic of the smoke collection setup is shown in FIG. 4. Smoke is drawn through several impingers connected in series to a pipe loaded with a cannabis pellet.

### Optimization of Cannabis Burning Protocol

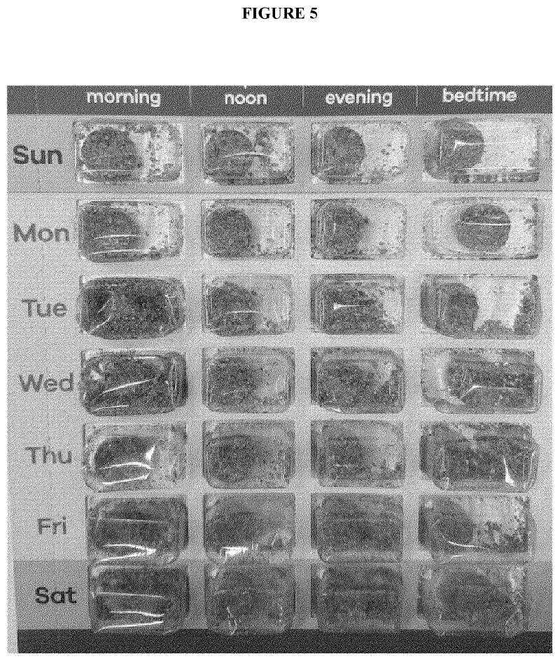

Prior to analyzing solvents for target analytes, several pellet-burning parameters were optimized. Many of the pellets received by RPC for testing were not fully intact (FIG. 5). Moreover, pellets that appeared intact upon first glance often fell apart during removal from the blister packaging, even with careful handling. Consequently, fragmented pellet material was used for both burning and vaporization experiments unless otherwise noted.

For vaporization experiments, the fragmented pellet material was loaded into the capsules that were provided with the Mighty vaporization device. Pellets from the same lot (lot #20180326@6) were used for all burn optimization experiments.

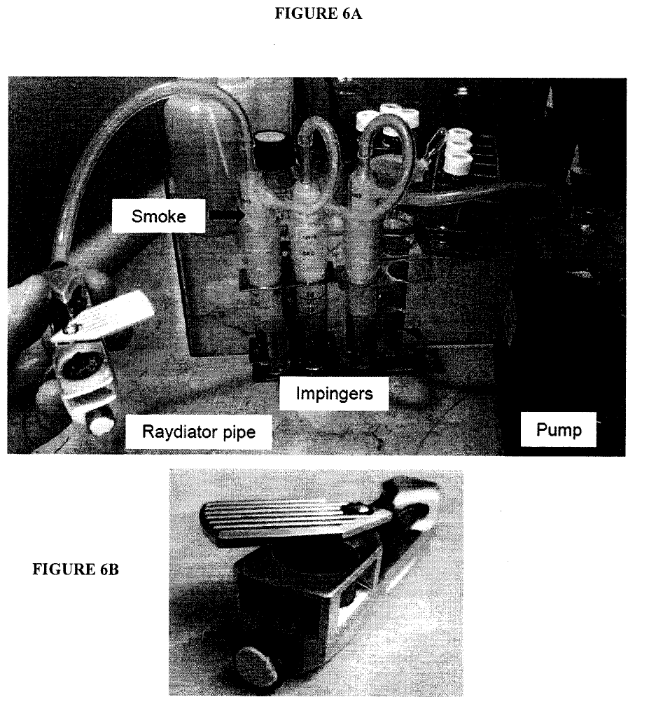

It was found that a flow rate of approximately 0.4 L/min was required for pellets to light in the pipe. At lower flow rates, there was insufficient draw through the impinger setup to successfully light the pellets. With a continuous pump flow (draw) rate of 0.4 L/min, pellets only needed to be lit once with a conventional flame lighter and typically burned very well, predominantly leaving only ash behind. Moreover, using a 0.4 L/min continuous draw generated predominantly mainstream smoke, with little or no sidestream smoke observed. Mainstream smoke can be seen migrating through the impinger platform in FIG. 6A. For all pipe experiments, a Teflon plug was used to block the side intake of the pipe (FIG. 6B).

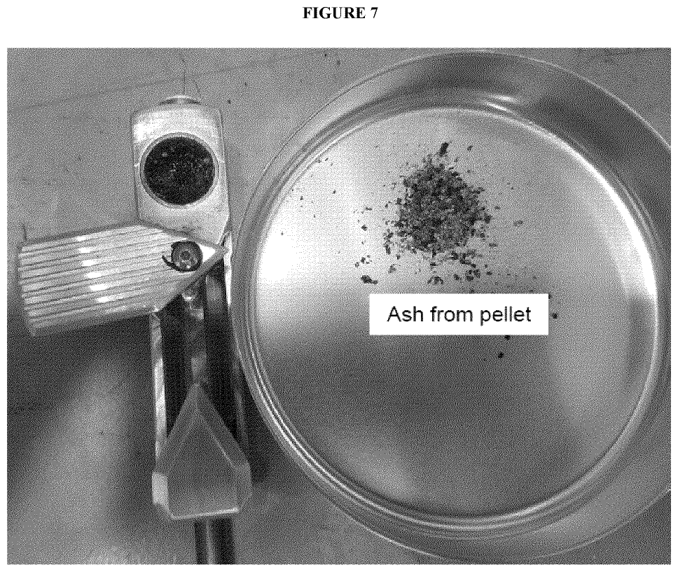

It was observed that pellets left exposed to ambient conditions (i.e. relative humidity of 40%-60%) for 24 hours did not burn as well as pellets that were removed from the blister packaging immediately prior to experiments. Pellets that burned very well had predominantly white ash remaining upon completion of the burn, with very little or no unburnt material observed (FIG. 7). For that reason, all smoke and vapor collection experiments were conducted within 30 minutes of pellet removal from the blister packaging.

It should be noted that the pump system used for these experiments was sufficient for a qualitative collection of smoke components. This is because the pump inadvertently had fluctuations in flow rate with no mechanism for mimicking a controlled puff scenario.

As mentioned previously, the purpose of experiments described in this report was to optimize a method for extraction of terpenes and cannabinoids, ultimately leading to future quantitative efforts. Controlled puff mimicking and quantitative experiments will be addressed in a subsequent project (Proposal No. AQS/18/05) via implementation of a SCIREQ inExpose smoking machine. Albeit, to obtain a “ball park” estimate of the efficiency of the extraction experiments conducted, cannabinoids and terpenes recovered from smoke and vapor experiments were compared to their respective values obtained from extracted plant material of the same lot. This approach is of course flawed since it assumes an unrealistic 100% conversion of analytes from plant material to smoke. Nevertheless, the calculations were conducted to provide some insight to levels observed in the smoke compared to direct extracts from the plant.

Cannabinoid and terpene values from pellet extracts (lot 20180326@6) were provided to RPC scientists by Tetra Bio-Pharma (Report ID: 282184-OAS). Report data from 282184-OAS is summarized in Tables 8 and 9.

TABLE 8
Cannabinoids from Extracted PPP001
Pellets (Lot 20180326@6, 282184-OAS)

| | Quantity in 280 mg unburnt |
|---|---|
| Analyte | pellet (% w/w) |
| Total Delta 9-THC (THC + THCA) | 8.81 |
| Total CBD (CBD + CBDA) | 2.61 |
| Total CBG (CBG + CBGA) | 0.37 |
| CBN | 0.15 |
| CBC | 0.07 |

TABLE 9
Terpenes from Extracted PPP001 Pellets
(Lot 20180326@6, 282184-OAS)

| | Quantity in 280 mg unburnt |
|---|---|
| Analyte | pellet (% w/w) |
| Linalool | 0.03 |
| Terpineol | 0.02 |
| Caryophyllene | 0.10 |
| Humulene | 0.04 |
| Fenchol | 0.01 |
| Borneol | 0.03 |
| Alpha-bisabolol | 0.02 |
| Total terpenes | 0.25 |

### Optimization of Impinger Solvent, Volumes, and Number of Impingers

Methanol was the first solvent evaluated for extraction of both cannabinoids and terpenes. Methanol was thought to be a good solvent because both the neutral and acidic forms of cannabinoids are known to be soluble in it. With respect to terpenes, which are non-polar compared to cannabinoids, methanol was considered less optimal. Albeit, initial extraction experiments investigated the feasibility of trapping both cannabinoids and terpenes with methanol.

Trial 1—Smoke Collection with 4 Impingers Containing Methanol

For Trial 1 experiments, smoke was collected using the parameters listed in Table 10. The relative recoveries from trapped smoke, calculated based on plant extract data (Table 8 and Table 9), are given in Table 11 (cannabinoids) and Table 12 (terpenes).

TABLE 10
Parameters for Trial 1

| | | |
|---|---|---|
| Mass of pellet | 347.7 | mg |
| Flow rate through pipe | 0.45 | L/min |

| | |
|---|---|
| Number of impingers | 4 |
| Solvent | Methanol |
| Solvent volume | 25 mL per impinger |

TABLE 11
Cannabinoid Results for Trial 1

| | Mass recovered from | |
|---|---|---|
| Analyte | smoke (mg)* | Relative recovery (%)** |
| Total Delta 9-THC (THC + THCA) | 11.73 | 38 |
| Total CBD (CBD + CBDA) | 3.43 | 38 |
| Total CBG (CBG + CBGA) | 0.43 | 33 |
| CBN | 0.70 | (134) |
| CBC | 0.33 | (134) |

*See Certificate of Analysis 282452-OAS (Appendix A).
**Calculated using unburnt pellet extract results as a benchmark.

TABLE 12
Terpene Results for Trial 1

| | Mass recovered from | |
|---|---|---|
| Analyte | smoke (mg)* | Relative recovery (%)** |
| Total Terpenes | 0.13 | 14 |
| Caryophyllene | 0.08 | 22 |
| Borneol | 0.05 | 48 |

*See Certificate of Analysis 282452-OAS (Appendix A).
**Calculated using unburnt pellet extract results as a benchmark.

As seen in Table 11, recoveries for total THC and total CBD were 38%, with THC followed by CBD as the most abundant cannabinoids recovered by mass. The relative THC recovery was about 14% lower than previously reported recoveries (52%) from cannabis cigarettes smoked under similar conditions (i.e. continuous pump draw of 0.5 L/min) [1]. Of the THC components recovered in the smoke, the majority (>99%) were in the neutral form, whereas for CBD components, 75% were in the neutral form. Temperatures during combustion should have been sufficiently high enough to convert all burned cannabinoids to their neutral (decarboxylated) forms. Since the combustion process is not 100% efficient, the cannabinoid acids detected may be from partially combusted material in the smoke particulate. Potential sources of analyte loss may include destruction from combustion process, adsorption on surfaces (i.e. tubing), and smoke escaping the trapping system. It was noted during these initial experiments that some smoke was still emanating from the last impinger. To improve efficiency of the impinger setup, subsequent experiments considered increasing solvent volume and/or adding impingers.

Interestingly, the relative recoveries of both CBN and CBC exceeded 100%. This may be due to bias in the calculation itself because of unreported CBNA and CBCA (acid forms of CBN and CBC). CBNA and CBCA are known to convert to CBN and CBC upon heating. Whereas THC, CBD, and CBG were analyzed for both the acid and neutral forms, this was not the case for CBN and CBC. Without also knowing the initial quantities of CBNA and CBCA in the dried pellet material, relative recovery values for CBN and CBC are not very meaningful. The relative recoveries for CBN and CBC are still included for completeness of the report but are placed in parentheses within tables throughout to distinguish them from the other cannabinoid recoveries.

With respect to terpenes (Table 12), only caryophyllene and borneol were present above the reporting limit and accounted for the entire 14% recovered. As expected, improvement of terpene recoveries most likely required a solvent less polar than methanol. Alternate solvents were explored in subsequent experiments which are presented below.

Cannabinoids in oil by HPLC

| | | | RPC Sample ID: | | | |
|---|---|---|---|---|---|---|
| | | | 282452-1 | 282452-2 | 282452-3 | 282452-4 |
| | | | Client Sample ID: | | | |
| | | | A01 | A02 | A03 | A04 |
| | | | Date Sampled: | | | |
| | | | 25 Jul. 2018 | 25 Jul. 2018 | 25 Jul. 2018 | 25 Jul. 2018 |
| | | | Matrix: | | | |
| Analysis | Units | RL | other | other | other | other |
| Delta 9-Tetrahydrocannabinol (Delta 9-THC) | mg/ml | 0.001 | 0.189 | 0.153 | 0.062 | 0.063 |
| Delta 9-Tetrahydrocannabinolic Acid (Delta 9-THCA) | mg/ml | 0.001 | [symbol] | [symbol] | ND | ND |
| Cannabidiol (CBD) | mg/ml | 0.001 | 0.047 | 0.039 | [symbol] | [symbol] |
| Cannabidiolic Acid (CBDA) | mg/ml | 0.001 | 0.012 | 0.007 | 0.002 | <0.001 |
| Cannabigerol (CBG) | mg/ml | 0.001 | 0.008 | 0.005 | 0.002 | 0.002 |
| Cannabigerolic Acid (CBGA) | mg/ml | 0.001 | <0.001 | ND | ND | ND |
| Cannabinol (CBN) | mg/ml | 0.001 | [symbol] | 0.009 | 0.004 | 0.004 |
| Cannabichromene (CBC) | mg/ml | 0.001 | [symbol] | 0.004 | 0.002 | 0.002 |
| Total Delta 9-Tetrahydrocannabinol (Delta 9-THC) | mg/ml | 0.001 | 0.191 | 0.153 | 0.062 | [symbol] |
| Total Cannabidiol (CBD) | mg/ml | 0.001 | 0.058 | 0.045 | 0.018 | 0.016 |
| Total Cannabigerol (CBG) | mg/ml | 0.001 | 0.008 | 0.005 | 0.002 | 0.002 |

This report relates only to the sample(s) and information provided to the laboratory.
Reported results are expressed on an as received basis.
RL = Reacting Limit,
ND = Not Detected [symbol]
indicates data missing or illegible when filed

Terpenes in cannabis oil

| | | | RPC Sample ID: | | | |
|---|---|---|---|---|---|---|
| | | | 282452-1 | 282452-2 | 282452-3 | 282452-4 |
| | | | Client Sample ID: | | | |
| | | | A01 | A02 | A03 | A04 |
| | | | Date Sampled: | | | |
| | | | 25 Jul. 2018 | 25 Jul. 2018 | 25 Jul. 2018 | 25 Jul. 2018 |
| | | | Matrix: | | | |
| Analytes | Units | RL | other | other | other | other |
| Alpha pinene | mg/mL | 0.001 | <0.001 | <0.001 | <0.001 | <0.001 |
| Beta pinene | mg/mL | 0.001 | <0.001 | <0.001 | <0.001 | <0.001 |
| Myrene | mg/mL | 0.001 | <0.001 | <0.001 | <0.001 | <0.001 |
| Limonene | mg/mL | 0.001 | <0.001 | <0.001 | <0.001 | <0.001 |
| Terpinolene | mg/mL | 0.001 | <0.001 | <0.001 | <0.001 | <0.001 |
| Linalool | mg/mL | 0.001 | <0.001 | <0.001 | <0.001 | <0.001 |
| Terpineol | mg/mL | 0.001 | <0.001 | <0.001 | <0.001 | <0.001 |
| Caryophylene | mg/mL | 0.001 | 0.002 | 0.001 | <0.001 | <0.001 |
| Humulene | mg/mL | 0.001 | <0.001 | <0.001 | <0.001 | <0.001 |
| 3-carene | mg/mL | 0.001 | <0.001 | <0.001 | <0.001 | <0.001 |
| Cis-ocimene | mg/mL | 0.001 | <0.001 | <0.001 | <0.001 | <0.001 |
| Eucalyptol | mg/mL | 0.001 | <0.001 | <0.001 | <0.001 | <0.001 |
| Trans-ocimene | mg/mL | 0.001 | <0.001 | <0.001 | <0.001 | <0.001 |
| Fenchol | mg/mL | 0.001 | <0.001 | <0.001 | <0.001 | <0.001 |
| Borneol | mg/mL | 0.001 | 0.001 | 0.001 | <0.001 | <0.001 |
| Valencene | mg/mL | 0.001 | <0.001 | <0.001 | <0.001 | <0.001 |
| Cis-nerolidol | mg/mL | 0.001 | <0.001 | <0.001 | <0.001 | <0.001 |
| Trans-nerolidol | mg/mL | 0.001 | <0.001 | <0.001 | <0.001 | <0.001 |
| Guaiol | mg/mL | 0.001 | <0.001 | <0.001 | <0.001 | <0.001 |
| Alpha-bisabolol | mg/mL | 0.001 | <0.001 | <0.001 | <0.001 | <0.001 |
| Sabinene | mg/mL | 0.001 | <0.001 | <0.001 | <0.001 | <0.001 |
| Total terpenes | mg/mL | 0.001 | 0.003 | 0.002 | <0.001 | <0.001 |

This report relates only the sample(s) and information promoted to the laboratory.
Reported results are expressed on an as received basis.
RL = Rapping Limit,
ND = Not Detected

Trial 2—Smoke Collection with 4 Impingers Containing Varying Solvents

In an attempt to improve both cannabinoid and terpene recoveries, another set of experiments (Trial 2) was designed in which 4 impingers were connected in series, with each impinge containing 50 mL of solvent. Each impinger was filled with a solvent of varying polarity and included the following: a) methanol; b) ethanol; c) 50:50 ethanol:hexane; d) 100% hexane.

The smoke collection parameters employed are those summarized in Table 13. Recovery results are presented in Tables 14 and 15.

TABLE 13
Smoke Collection Parameters for Trial 2

| | | |
|---|---|---|
| Mass of pellet | 306.3 | mg |
| Flow rate through pipe | 0.39 | L/min |

| | |
|---|---|
| Number of impingers | 4 |
| Solvents | Methanol |
| | Ethanol |
| | 50:50 ethanol:hexane mixture |
| | Hexane |
| Solvent volume | 50 mL per impinger |

TABLE 14
Cannabinoid Results for Trial 2

| | Mass recovered from | |
|---|---|---|
| Analyte | smoke (mg)* | Relative recovery (%)** |
| Total Delta 9-THC (THC + THCA) | 9.30 | 34 |
| Total CBD (CBD + CBDA) | 2.90 | 36 |
| Total CBG (CBG + CBGA) | 0.25 | 22 |
| CBN | 0.50 | (109) |
| CBC | 0.20 | (93) |

*See Certificate of Analysis 284316-OAS (Appendix B).
**Calculated using unburnt pellet extract results as a benchmark.

TABLE 15
Terpene Results for Trial 2

| | Mass recovered from | |
|---|---|---|
| Analyte | smoke (mg)* | Relative recovery (%)** |
| Total Terpenes | 0.10 | 13 |
| Caryophyllene | 0.10 | 33 |

*See Certificate of Analysis 284316-OAS (Appendix B).
**Calculated using unbumt pellet extract results as a benchmark.

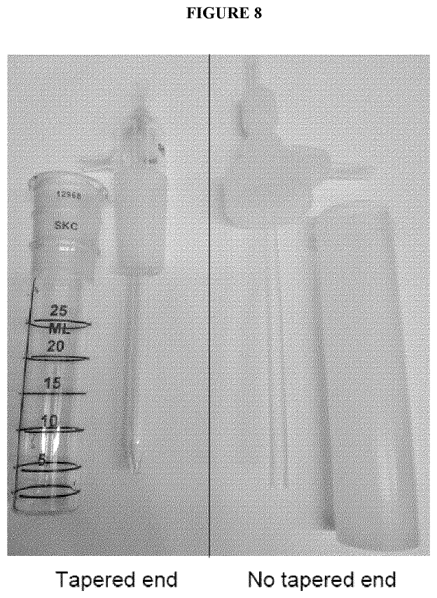

As seen in Tables 14 and 15, using larger impingers with more solvent did not improve recoveries of cannabinoids and terpenes compared to Trial 1. As a matter of fact, recoveries for all analytes were slightly lower. This may be due to a difference in impinger shape: the smaller impingers used in Trial 1 had stems with tapered ends, whereas the larger impingers used in Trial 2 did not have tapered ends (FIG. 8). Cannabinoids were present in all four impingers with recoveries comparable to the first trial. Because increasing impinger volume did not significantly improve recoveries, the smaller impingers (tapered stem ends) were used for all subsequent experiments. Use of smaller impingers containing less solvent may be advantageous since it reduces the degree to which trapped components are diluted by the solvent. As one can imagine, this is particularly important for the terpenes, which are only present in the cannabis pellets at low concentrations to begin with. Unlike in Trial 1, where caryophyllene and borneol were detected, only caryophyllene was detected in Trial 2, indicating that the increase in solvent volume diluted the terpene to a non-detectable quantity.

Cannabinoids in oil by HPLC

| | | | RPC Sample ID: | | | |
|---|---|---|---|---|---|---|
| | | | 284316-1 | 284316-2 | 284316-3 | 284316-4 |
| | | | Client Sample ID: | | | |
| | | | B01 | B02 | B03 | B04 |
| | | | Date Sampled: | | | |
| | | | 8 Aug. 2018 | 8 Aug. 2018 | 8 Aug. 2018 | 8 Aug. 2018 |
| | | | Matrix: | | | |
| Analytes | Units | RL | other | other | other | other |
| Delta 9-Tetrahydrocannabinol (Delta 9-THC) | mg/ml | 0.001 | 0.060 | 0.053 | 0.044 | 0.014 |
| Delta 9-Tetrahydrocannabinolic Acid (Delta 9-THCA) | mg/ml | 0.001 | ND | ND | ND | ND |
| Cannabidiol (CBD) | mg/ml | 0.001 | 0.015 | 0.013 | 0.011 | 0.004 |
| Cannabidiolic Acid (CBDA) | mg/ml | 0.001 | 0.010 | 0.002 | ND | ND |
| Cannabigerol (CBG) | mg/ml | 0.001 | 0.002 | 0.002 | 0.001 | ND |
| Cannabigerolic Acid (CBGA) | mg/ml | 0.001 | ND | ND | ND | ND |
| Cannabinol (CBN) | mg/ml | 0.001 | 0.001 | 0.004 | 0.003 | 0.001 |
| Cannabichromene (CBC) | mg/ml | 0.001 | 0.002 | 0.001 | 0.001 | ND |
| Total Delta 9-Tetrahydrocannabinol (Delta 9-THC) | mg/ml | 0.001 | 0.061 | 0.053 | 0.044 | 0.014 |
| Total Cannabidiol (CBD) | mg/ml | 0.001 | 0.024 | 0.015 | 0.011 | 0.004 |
| Total Cannabigerol (CBG) | mg/ml | 0.001 | 0.002 | 0.002 | 0.001 | ND |

This report relates only to the sample(s) and information provided to the laboratory.
Reported results are expressed on an as received basis.
RL = Reporting Limit,
ND = Not Detected

Terpenes in cannabis oil

| | | | RPC Sample ID: | | | |
|---|---|---|---|---|---|---|
| | | | 284316-1 | 284316-2 | 284316-3 | 284316-4 |
| | | | Client Sample ID: | | | |
| | | | B01 | B02 | B03 | B04 |
| | | | Date Sampled: | | | |
| | | | 8 Aug. 2018 | 8 Aug. 2018 | 8 Aug. 2018 | 8 Aug. 2018 |
| | | | Matrix: | | | |
| Analytes | Units | RL | other | other | other | other |
| Alpha pinene | % | 0.001 | <0.001 | <0.001 | <0.001 | <0.001 |
| Beta pinene | % | 0.001 | <0.001 | <0.001 | <0.001 | <0.001 |
| Myrcene | % | 0.001 | <0.001 | <0.001 | <0.001 | <0.001 |
| Limonene | % | 0.001 | <0.001 | <0.001 | <0.001 | <0.001 |
| Terpinolene | % | 0.001 | <0.001 | <0.001 | <0.001 | <0.001 |
| Linalool | % | 0.001 | <0.001 | <0.001 | <0.001 | <0.001 |
| Terpineol | % | 0.001 | <0.001 | <0.001 | <0.001 | <0.001 |
| Caryophylene | % | 0.001 | 0.002 | <0.001 | <0.001 | <0.001 |
| Humulene | % | 0.001 | <0.001 | <0.001 | <0.001 | <0.001 |
| 3-carene | % | 0.001 | 0.001 | <0.001 | <0.001 | <0.001 |
| Cis-ocimene | % | 0.001 | <0.001 | <0.001 | <0.001 | <0.001 |
| Eucalyptol | % | 0.001 | <0.001 | <0.001 | <0.001 | <0.001 |
| Trans-ocimene | % | 0.001 | <0.001 | <0.001 | <0.001 | <0.001 |
| Fenchol | % | 0.001 | <0.001 | <0.001 | <0.001 | <0.001 |
| Borneol | % | 0.001 | <0.001 | <0.001 | <0.001 | <0.001 |

This report relates only to the sample(s) and information provided to the laboratory.
Reported results are expressed on an as received basis.
RL = Reporting Limit,
ND = Not Detected

Terpenes in cannabis oil

| | | | RPC Sample ID: | | | |
|---|---|---|---|---|---|---|
| | | | 284316-1 | 284316-2 | 284316-3 | 284316-4 |
| | | | Client Sample ID: | | | |
| | | | B01 | B02 | B03 | B04 |
| | | | Date Sampled: | | | |
| | | | 8 Aug. 2018 | 8 Aug. 2018 | 8 Aug. 2018 | 8 Aug. 2018 |
| | | | Matrix: | | | |
| Analytes | Units | RL | other | other | other | other |
| Valencene | % | 0.001 | <0.001 | <0.001 | <0.001 | <0.001 |
| Cis-nerolidol | % | 0.001 | <0.001 | <0.001 | <0.001 | <0.001 |
| Trans-nerolidol | % | 0.001 | <0.001 | <0.001 | <0.001 | <0.001 |
| Guaiol | % | 0.001 | <0.001 | <0.001 | <0.001 | <0.001 |
| Alpha-bisabolol | % | 0.001 | <0.001 | <0.001 | <0.001 | <0.001 |
| Sabinene | % | 0.001 | <0.001 | <0.001 | <0.001 | <0.001 |
| Total terpenes | % | 0.001 | 0.002 | <0.001 | <0.001 | <0.001 |

This report relates only to the sample(s) and information provided to the laboratory.
Reported results are expressed on an as received basis.
RL = Reporting Limit,
ND = Not Detected

Trail 3—Effects of Solvents on Terpene Recoveries from a Gas Standard

To improve on low terpene recoveries, two subsequent experiments were designed to look at the effects of solvent (Trial 3) and temperature (Trial 4) on terpene recoveries. These experiments were conducted using a terpene gas standard that contained 10 ppmv of each of the following components: a) Alpha pinene; b) Beta pinene; c) 3-carene; d) Limonene. A known volume of terpene gas standard was bubbled through an impinger containing each of three solvents in order of decreasing polarity. The solvents were ethanol, 50:50 ethanol:hexane, and hexane (Table 16). By knowing the volume and concentration of the standard used, recovery of terpenes trapped in each impinger could be calculated (Table 17).

Both the 50:50 mixture and pure hexane yielded equivalent recoveries of total terpenes. These results were in contrast to pure ethanol, for which no terpenes were reported. Based on these results, hexane was selected as the optimal solvent for all subsequent terpene trapping experiments.

TABLE 16
Terpene Gas Standard Collection Parameters for Trial 3

| | |
|---|---|
| Total mass of terpenes from | 0.512 mg (ethanol) |
| standard | 0.528 mg (50:50 ethanol:hexane) |
| | 0.522 mg (hexane) |
| Flow rate through impinger | 0.078 L/min |
| Number of impingers | 1 |
| Solvents | Ethanol |
| | or |
| | 50:50 ethanol:hexane mixture |
| | or |
| | Hexane |
| Solvent volume | 25 mL per impinger |

TABLE 17
Total Terpene Recoveries for Trial 3

| Solvent | Total terpene recovery (%)* |
|---|---|
| Ethanol | 0 |
| 50:50 ethanolhexane mixture | 19 |
| Hexane | 19 |

*See Certificate of Analysis 286508-OAS Rev01 (Appendix C).

Terpenes in cannabis oil

| | | | RPC Sample ID: | | |
|---|---|---|---|---|---|
| | | | 286508-1 | 286508-2 | 286508-3 |
| | | | Client Sample ID: | | |
| | | | C01 | C02 | C03 |
| | | | terpenes in | terpenes in | terpenes in |
| | | | ethanolhexane | ethanol | hexane |
| | | | Date Sampled: | | |
| | | | 23 Aug. 2018 | 23 Aug. 2018 | 23 Aug. 2018 |
| | | | Matrix: | | |
| Analytes | Units | RL | other | other | other |
| Alpha pinene | % | 0.0001 | 0.0002 | <0.0001 | 0.0002 |
| Beta pinene | % | 0.0001 | 0.0002 | <0.0001 | 0.0002 |
| Myrcene | % | 0.0001 | <0.0001 | <0.0001 | <0.0001 |
| Limonene | % | 0.0001 | <0.0001 | <0.0001 | <0.0001 |
| Terpinolene | % | 0.0001 | <0.0001 | <0.0001 | <0.0001 |
| Linalool | % | 0.0001 | <0.0001 | <0.0001 | <0.0001 |
| Terpineol | % | 0.0001 | <0.0001 | <0.0001 | <0.0001 |
| Caryophylene | % | 0.0001 | <0.0001 | <0.0001 | <0.0001 |
| Humulene | % | 0.0001 | <0.0001 | <0.0001 | <0.0001 |
| 3-carene | % | 0.0001 | <0.0001 | <0.0001 | <0.0001 |
| Cis-ocimene | % | 0.0001 | <0.0001 | <0.0001 | <0.0001 |
| Eucalyptol | % | 0.0001 | <0.0001 | <0.0001 | <0.0001 |
| Trans-ocimene | % | 0.0001 | <0.0001 | <0.0001 | <0.0001 |
| Fenchol | % | 0.0001 | <0.0001 | <0.0001 | <0.0001 |
| Borneol | % | 0.0001 | <0.0001 | <0.0001 | <0.0001 |

This report relates only to the sample(s) and information provided to the laboratory.
Reported results are expressed on an as received basis.
RL = Reporting Limit, ND = Not Detected

Terpenes in cannabis oil

| | | | RPC Sample ID: | | |
|---|---|---|---|---|---|
| | | | 286508-1 | 286508-2 | 286508-3 |
| | | | Client Sample ID: | | |
| | | | C01 | C02 | C03 |
| | | | terpenes in | terpenes in | terpenes in |
| | | | ethanolhexane | ethanol | hexane |
| | | | Date Sampled: | | |
| | | | 23 Aug. 2018 | 23 Aug. 2018 | 23 Aug. 2018 |
| | | | Matrix: | | |
| Analytes | Units | RL | other | other | other |
| Valencene | % | 0.0001 | <0.0001 | <0.0001 | <0.0001 |
| Cis-nerolidol | % | 0.0001 | <0.0001 | <0.0001 | <0.0001 |
| Trans-nerolidol | % | 0.0001 | <0.0001 | <0.0001 | <0.0001 |
| Guaiol | % | 0.0001 | <0.0001 | <0.0001 | <0.0001 |
| Alpha-bisabolol | % | 0.0001 | <0.0001 | <0.0001 | <0.0001 |
| Sabinene | % | 0.0001 | <0.0001 | <0.0001 | <0.0001 |
| Total terpenes | % | 0.0001 | 0.0004 | <0.0001 | 0.0006 |

Trial 4—Effect of Temperature on Terpene Recoveries from a Gas Standard

Using hexane as the desired solvent, Trial 4 experiments investigated the effect of temperature on terpene trapping efficiency. A known volume of terpene gas standard was again bubbled through an impinger containing hexane at either room temperature or submersed in a cryogenic bath (Table 18). Recoveries of total terpenes were calculated and are shown in Table 19.

TABLE 18
Terpene Gas Standard Collection Parameters for Trial 4

| | |
|---|---|
| Total mass of terpenes from | 0.526 mg (Room) |
| standard | 0.529 mg (Cryogenic) |
| Flow rate through impinger | 0.079 L/min |
| Number of impingers | 1 |
| Impinger temperature | Room (24° C.) |
| | or |
| | Cryogenic (−70° C.) |
| Solvent | Hexane |
| Solvent volume | 25 mL per impinger |

TABLE 19
Total Terpene Recoveries for Trial 4

| Temperature of solvent in impinger | Total terpene recovery (%)* |
|---|---|
| Room, 24° C. | 19 |
| Cryogenic, −70° C. | 24 |

*See Certificate of Analysis 289829-OAS (Appendix D).

Only a modest increase in terpene recovery was observed when the terpene gas standard was bubbled through an impinger under cryogenic conditions. Given the added complexity of the cryogenic trapping system, room temperature conditions were deemed suitable for trapping of terpenes.

Terpenes in cannabis oil

| | | | RPC Sample ID: | |
|---|---|---|---|---|
| | | | 289829-1 | 289829-2 |
| | | | Client Sample ID: | |
| | | | F01 (Cannabis | F02 (Cannabis |
| | | | Extract in | Extract in |
| | | | Hexanes) | Hexanes) |
| | | | Date Sampled: | |
| | | | 19 Sep. 2018 | 19 Sep. 2018 |
| | | | Matrix: | |
| Analytes | Units | RL | other | other |
| Alpha pinene | mg/mL | 0.001 | 0.002 | 0.003 |
| Beta pinene | mg/mL | 0.001 | 0.002 | 0.002 |
| Myrcene | mg/mL | 0.001 | <0.001 | <0.001 |
| Limonene | mg/mL | 0.001 | <0.001 | <0.001 |
| Terpinolene | mg/mL | 0.001 | <0.001 | <0.001 |
| Linalool | mg/mL | 0.001 | <0.001 | <0.001 |
| Terpineol | mg/mL | 0.001 | <0.001 | <0.001 |
| Caryophylene | mg/mL | 0.001 | <0.001 | <0.001 |
| Humulene | mg/mL | 0.001 | <0.001 | <0.001 |
| 3-carene | mg/mL | 0.001 | <0.001 | <0.001 |
| Cis-ocimene | mg/mL | 0.001 | <0.001 | <0.001 |
| Eucalyptol | mg/mL | 0.001 | <0.001 | <0.001 |
| Trans-ocimene | mg/mL | 0.001 | <0.001 | <0.001 |
| Fenchol | mg/mL | 0.001 | <0.001 | <0.001 |
| Borneol | mg/mL | 0.001 | <0.001 | <0.001 |
| Valencene | mg/mL | 0.001 | <0.001 | <0.001 |
| Cis-nerolidol | mg/mL | 0.001 | <0.001 | <0.001 |
| Trans-nerolidol | mg/mL | 0.001 | <0.001 | <0.001 |
| Guaiol | mg/mL | 0.001 | <0.001 | <0.001 |
| Alpha-bisabolol | mg/mL | 0.001 | <0.001 | <0.001 |
| Sabinene | mg/mL | 0.001 | <0.001 | <0.001 |
| Total terpenes | mg/mL | 0.001 | 0.004 | 0.005 |

This report relates only to the sample(s) and information provided to the laboratory. [symbol]
ported results are expressed on an as received basis. [symbol]
= Reporting Limit, ND = Not Detected [symbol]
indicates data missing or illegible when filed

### Trial 5—Collection of Cannabis Smoke Using an Optimized Impinger Trapping System

Results from the first 4 trials were used to optimize the impinger trapping system for cannabis smoke. Favorable results were obtained for cannabinoids when using methanol as a solvent, although even with four impingers, breakthrough (i.e. analytes escaping from the last impinger) was still observed. For this reason, a fifth impinger was added to the series in an attempt to improve cannabinoid trapping. Given that concentrations of trapped cannabinoids were well above reporting limits, volumes of 25 mL were maintained.

With respect to terpenes, experiments with the terpene gas standard (Table 19) suggested that non-polar solvents such as hexane were a better solvent choice than more polar solvents such as ethanol or methanol. Consequently, terpene extraction from cannabis smoke should be conducted separately from cannabinoid extraction. Moreover, because the concentration of trapped terpenes was very close to the reporting limits when 25 mL of solvent was used, lower volumes of solvent (10 mL per impinger) were explored for terpene trapping in order to maximize detection of the analytes. Furthermore, terpenes were only observed in the first 2 impingers in previous experiments, thus a total of 3 impingers was deemed feasible to trap terpenes.

To summarize, the optimized collection platform for cannabis smoke consisted of two independent trapping systems at room temperature: one for trapping of terpenes and the other for trapping of cannabinoids. More specifically, five impingers in series (each containing 25 mL of methanol) for trapping of cannabinoids and three impingers in series (each containing 10 mL of hexane) for trapping of terpenes were used (Table 20).

In order to maximize recoveries, all impingers and tubing were rinsed with the applicable solvent from the impingers. Recoveries for cannabinoids and terpenes are presented in Tables 21 and 22.

TABLE 20
Smoke Collection Parameters for Trial 5

| | |
|---|---|
| Mass of pellets | 256.0 mg (for cannabinoid trapping) |
| | 273.6 mg (for terpene trapping) |
| Flow rate through pipe | 0.53 L/min for cannabinoid trapping |
| | 0.54 L/min for terpene trapping |
| Number of impingers | 5 for cannabinoid trapping |
| | 3 for terpene trapping |
| Solvents/volumes | Methanol, 25 mL per impinger, for cannabinoid trapping |
| | Hexane, 10 mL per impinger, for terpene trapping |

TABLE 21
Cannabinoid Results for Trial 5

| | Mass recovered from | |
|---|---|---|
| Analyte | smoke (mg)* | Relative recovery (%)** |
| Total Delta 9-THC (THC + THCA) | 10.78 | 48 |
| Total CBD (CBD + CBDA) | 2.68 | 40 |
| Total CBG (CBG + CBGA) | 0.33 | 34 |
| CBN | 0.98 | (254) |
| CBC | 0.25 | (140) |

*See Certificate of Analysis 293474-OAS (Appendix E).
**Calculated using unburnt pellet extract results as a benchmark.

TABLE 22
Terpene Results for Trial 5

| | Mass recovered from | |
|---|---|---|
| Analyte | smoke (mg)* | Relative recovery (%)** |
| Total Terpenes | 0.02 | 3 |
| Caryophyllene | 0.01 | 4 |
| Alpha-bisabolol | 0.01 | 18 |

*See Certificate of Analysis 293474-OAS (Appendix E).
**Calculated using unburnt pellet extract results as a benchmark.

As seen in Table 21, five impingers increased the recoveries for THC and CBD compared to Trials 1 and 2. Moreover, a recovery of48% for THC is consistent with recoveries reported in literature (52%) when cannabis cigarettes are smoked under similar conditions (i.e. continuous pump draw of 0.5 L/min) [1]. Some THC was still detected in the fifth impinger, however the quantity of THC detected accounted for less than 6% of the recovered mass.

Despite improvement in cannabinoid recoveries, the total terpene recoveries (Table 22) were lower than previous trials, with only caryophyllene and alpha-bisabolol being detected. One possible explanation for this may be that the combustion process is converting some terpenes into other molecular components, such as methacrolein [2]. Interestingly, methacrolein was detected as a volatile organic compound constituent in cannabis smoke generated from PPPOO1 pellets in previous experiments (see RPC report AQS-J1314). Also, it could be possible that slight differences in flow rates between each of the trials could be influencing the burn temperature and trapping efficiency. For example, a higher flow rate can lead to a higher burn temperature, which could result in more terpene degradation. A higher flow rate can also increase the degree of bubbling in the impingers, which can lead to more solvent and trapped terpene evaporation. A more controlled pump system would mitigate these factors and will be explored in detail with the SCJREQ inExpose smoking machine.

Terpene recovery may also be influenced by variations in the pellets themselves. A larger set of pellets would need to be analyzed in a repeatable manner to draw conclusions on variability between the pellets.

Cannabinoids in oil by HPLC

| | | | RPC Sample ID: | | | | |
|---|---|---|---|---|---|---|---|
| | | | 293474-4 | 293474-5 | 293474-6 | 293474-7 | 293474-8 |
| | | | Client Sample ID: | | | | |
| | | | K11 | K12 | K13 | K14 | K15 |
| | | | (Cannabis | (Cannabis | (Cannabis | (Cannabis | (Cannabis |
| | | | Smoke In | Smoke In | Smoke In | Smoke In | Smoke In |
| | | | Methanol) | Methanol) | Methanol) | Methanol) | Methanol) |
| | | | Date Sampled: | | | | |
| | | | 19 Oct. 2018 | 19 Oct. 2018 | 19 Oct. 2018 | 19 Oct. 2018 | 19 Oct. 2018 |
| | | | Matrix: | | | | |
| Analytes | Units | RL | other | other | other | other | other |
| Delta 9-Tetrahydrocannabinol (Delta 9-THC) | mg/ml | 0.001 | 0.116 | 0.163 | 0.089 | 0.039 | 0.024 |
| Delta 9-Tetrahydrocannabinolic | mg/ml | 0.001 | <0.001 | ND | ND | ND | ND |
| Acid (Delta 9-THC [symbol] | | | | | | | |
| Cannabidiol (CBD) | mg/ml | 0.001 | 0.029 | 0.040 | 0.021 | 0.011 | 0.006 |
| Cannabidiolic Acid (CBDA) | mg/ml | 0.001 | 0.001 | ND | <0.001 | ND | ND |
| Cannabigerol (CBG) | mg/ml | 0.001 | 0.004 | 0.006 | 0.003 | <0.001 | ND |
| Cannabigerolic Acid (CBGA) | mg/ml | 0.001 | ND | ND | ND | ND | ND |
| Cannabinol (CBN) | mg/ml | 0.001 | 0.010 | 0.014 | 0.008 | 0.004 | 0.003 |
| Cannabichromene (CBC) | mg/ml | 0.001 | 0.003 | 0.005 | 0.002 | ND | ND |
| Total Delta 9-Tetrahydrocannabinol | mg/ml | 0.001 | 0.116 | 0.163 | 0.089 | 0.039 | 0.024 |
| (Delta 9-THC [symbol] | | | | | | | |
| Total Cannabidiol (CBD) | mg/ml | 0.001 | 0.029 | 0.040 | 0.021 | 0.011 | 0.006 |
| Total Cannabigerol (CBG) | mg/ml | 0.001 | 0.004 | 0.006 | 0.003 | <0.001 | ND |

This report relates only to the sample(s) and information provided to the laboratory.
Reported results are expressed on an as received basis.
RL = Reporting Limit,
ND = Not Detected [symbol]
indicates data missing or illegible when filed

Terpenes in cannabis oil

| | | | RPC Sample ID: | | |
|---|---|---|---|---|---|
| | | | 293474-1 | 293474-2 | 283474-3 |
| | | | Client Sample ID: | | |
| | | | K01 | K02 | K03 |
| | | | (Cannabis Smoke | (Cannabis Smoke | (Cannabis Smoke |
| | | | In Hexane) | In Hexane) | In Hexane) |
| | | | Date Sampled: | | |
| | | | 19 Oct. 2018 | 19 Oct. 2018 | 19 Oct. 2018 |
| | | | Matrix: | | |
| Analytes | Units | RL | other | other | other |
| Alpha pinene | mg/mL | 0.001 | <0.001 | <0.001 | <0.001 |
| Beta pinene | mg/mL | 0.001 | <0.001 | <0.001 | <0.001 |
| Myrcene | mg/mL | 0.001 | <0.001 | <0.001 | <0.001 |
| Limonene | mg/mL | 0.001 | <0.001 | <0.001 | <0.001 |
| Terpinolene | mg/mL | 0.001 | <0.001 | <0.001 | <0.001 |
| Linalool | mg/mL | 0.001 | <0.001 | <0.001 | <0.001 |
| Terpineol | mg/mL | 0.001 | <0.001 | <0.001 | <0.001 |
| Caryophylene | mg/mL | 0.001 | <0.001 | 0.001 | <0.001 |
| Humulene | mg/mL | 0.001 | <0.001 | <0.001 | <0.001 |
| 3-carene | mg/mL | 0.001 | <0.001 | <0.001 | <0.001 |
| Cis-ocimene | mg/mL | 0 001 | <0.001 | <0.001 | <0.001 |
| Eucalyptol | mg/mL | 0.001 | <0.001 | <0.001 | <0.001 |
| Trans-ocimene | mg/mL | 0.001 | <0.001 | <0.001 | <0.001 |
| Fenchol | mg/mL | 0.001 | <0.001 | <0.001 | <0.001 |
| Bomeol | mg/mL | 0.001 | <0.001 | <0.001 | <0.001 |
| Valencene | mg/mL | 0.001 | <0.001 | <0.001 | <0.001 |
| Cis-nerolidol | mg/mL | 0.001 | <0.001 | <0.001 | <0.001 |
| Trans-nerolidol | mg/mL | 0.001 | <0.001 | <0.001 | <0.001 |
| Guaiol | mg/mL | 0.001 | <0.001 | <0.001 | <0.001 |
| Alpha-bisabolol | mg/mL | 0.001 | <0.001 | 0.001 | <0.001 |
| Sabinene | mg/mL | 0.001 | <0.001 | <0.001 | <0.001 |
| Total terpenes | mg/mL | 0.001 | <0.001 | 0.002 | <0.001 |

This report relates only to the sample(s) and information provided to the laboratory.
Reported results are expressed on an as received basis.
RL = Reporting Limit, ND = Not Detected

### Trial 6—Evaluation of Trapping Efficiency of Impinger System Using a Terpene Gas Standard

The impinger-based trapping system for terpenes developed in the previous trial was evaluated for trapping efficiency (Trial 6) using a terpene gas standard. The terpene gas standard, which was also used for Trials 3 and 4, contained 10 ppmv of each of the following components: a) Alpha pinene; b) Beta pinene; c) 3-carene; d) Limonene.

These experiments were performed in duplicate (Run 1 and Run 2) using flow rates in the same range as those employed for smoke and vapor collection experiments. Collection parameters for this set of experiments are presented in Table 23 with results summarized in Table 24.

TABLE 23
Terpene Gas Standard Collection Parameters for Trial 6

| | |
|---|---|
| Total terpene concentration | 0.223 mg/L |
| of gas standard | |
| Volume of standard used | 2.54 L (Run 1) |
| | 2.64 L (Run 2) |
| Flow rate through impingers | 0.51 L/min (Run 1) |
| | 0.53 L/min (Run 2) |
| Number of impingers | 3 |
| Solvent | Hexane |
| Solvent volume | 10 mL per impinger |

TABLE 24
Terpene Gas Standard Collection Results for Trial 6

| | Run 1* | | Run 2* | |
|---|---|---|---|---|
| Analyte | Mass (mg) | Recovery (%) | Mass (mg) | Recovery (%) |
| Total Terpenes | 0.42 | 74 | 0.41 | 70 |
| Alpha pinene | 0.11 | 78 | 0.12 | 81 |
| Beta pinene | 0.12 | 85 | 0.11 | 75 |
| Limonene | 0.10 | 71 | 0.09 | 61 |
| 3-carene | 0.09 | 63 | 0.09 | 61 |

*See Certificate of Analysis 293340-OAS Rev01 (Appendix F)

The total terpenes recovered by the impinger system was very good, with an average recovery of 72% between the duplicate runs. In both experiments, all detected terpenes were found in the first impinger, with no terpenes detected in subsequent impingers. These results indicate that the impinger setup is quite efficient for trapping the terpenes.

Unfortunately, due to the lack of availability of a cannabinoid gas standard, the same experiment could not be conducted to determine the recovery of cannabinoids. For that reason, only the relative recoveries can be used to judge trapping efficiency of cannabinoids and for reasons previously mentioned, should be interpreted with caution since 100% conversion of analytes between unburned material and burned material is unlikely.

Terpenes in cannabis oil

| | | | RPC Sample ID: | | | | |
|---|---|---|---|---|---|---|---|
| | | | 293340-1 | 293340-2 | 293340-3 | 293340-4 | 293340-5 |
| | | | Client Sample ID: | | | | |
| | | | J01 (Cannabis | J02 (Cannabis | J03 (Cannabis | J11 (Cannabis | J12 (Cannabis |
| | | | Extract in | Extract in | Extract in | Extract in | Extract in |
| | | | Hexanes) | Hexanes) | Hexanes) | Hexanes) | Hexanes) |
| | | | Date Sampled: | | | | |
| | | | 18 Oct. 2018 | 18 Oct. 2018 | 18 Oct. 2018 | 18 Oct. 2018 | 18 Oct. 2018 |
| | | | Matrix: | | | | |
| Analytes | Units | RL | other | other | other | other | other |
| Alpha pinene | % | 0.001 | 0.011 | <0.001 | <0.001 | 0.012 | <0.001 |
| Beta pinene | % | 0.001 | 0.012 | <0.001 | <0.001 | 0.011 | <0.001 |
| Myrcene | % | 0.001 | <0.001 | <0.001 | <0.001 | <0.001 | <0.001 |
| Limonene | % | 0.001 | 0.010 | <0.001 | <0.001 | 0.009 | <0.001 |
| Terpinolene | % | 0.001 | <0.001 | <0.001 | <0.001 | <0.001 | <0.001 |
| Linalool | % | 0.001 | <0.001 | <0.001 | <0.001 | <0.001 | <0.001 |
| Terpineol | % | 0.001 | <0.001 | <0.001 | <0.001 | <0.001 | <0.001 |
| Caryophylene | % | 0.001 | <0.001 | <0.001 | <0.001 | <0.001 | <0.001 |
| Humulene | % | 0.001 | <0.003 | <0.001 | <0.001 | <0.001 | <0.001 |
| 3-carene | % | 0.001 | 0.009 | <0.001 | <0.001 | 0.009 | <0.001 |
| Cis-ocimene | % | 0.001 | <0.001 | <0.001 | <0.001 | <0.001 | <0.001 |
| Eucalyptol | % | 0.001 | <0.001 | <0.001 | <0.001 | <0.001 | <0.001 |
| Trans-ocimene | % | 0.001 | <0.001 | <0.001 | <0.001 | <0.001 | <0.001 |
| Fenchol | % | 0.001 | <0.001 | <0.001 | <0.001 | <0.001 | <0.001 |
| Borneol | % | 0.001 | <0.001 | <0.001 | <0.001 | <0.001 | <0.001 |

This report relates only to the sample(s) and information provided to the laboratory.
Reported results are expressed on an as received basis.
RL = Reporting Limit,
ND = Not Detected

Terpenes in cannabis oil

| | | | RPC Sample ID: | | | | |
|---|---|---|---|---|---|---|---|
| | | | 293340-1 | 293340-2 | 293340-3 | 293340-4 | 293340-5 |
| | | | Client Sample ID: | | | | |
| | | | J01 (Cannabis | J02 (Cannabis | J03 (Cannabis | J11 (Cannabis | J12 (Cannabis |
| | | | Extract in | Extract in | Extract in | Extract in | Extract in |
| | | | Hexanes) | Hexanes) | Hexanes) | Hexanes) | Hexanes) |
| | | | Date Sampled: | | | | |
| | | | 18 Oct. 2018 | 18 Oct. 2018 | 18 Oct. 2018 | 18 Oct. 2018 | 18 Oct. 2018 |
| | | | Matrix: | | | | |
| Analytes | Units | RL | other | other | other | other | other |
| Valencene | % | 0.001 | <0.001 | <0.001 | <0.001 | <0.001 | <0.001 |
| Cis-nerolidol | % | 0.001 | <0.001 | <0.001 | <0.001 | <0.001 | <0.001 |
| Trans-nerolidol | % | 0.001 | <0.001 | <0.001 | <0.001 | <0.001 | <0.001 |
| Guaiol | % | 0.001 | <0.001 | <0.001 | <0.001 | <0.001 | <0.001 |
| Alpha-bisabolol | % | 0.001 | <0.001 | <0.001 | <0.001 | <0.001 | <0.001 |
| Sabinene | % | 0.001 | <0.001 | <0.001 | <0.001 | <0.001 | <0.001 |
| Total terpenes | % | 0.001 | 0.032 | <0.001 | <0.001 | 0.052 | <0.001 |

This report relates only to the sample(s) and information provided to the laboratory.
Reported results are expressed on an as received basis.
RL = Reporting Limit,
ND = Not Detected

Terpenes in cannabis oil

| | | | RPC Sample ID: |
|---|---|---|---|
| | | | 293340-6 |
| | | | Client Sample ID: |
| | | | J13 (Cannabis |
| | | | Extract in |
| | | | Hexanes) |
| | | | Date Sampled: |
| | | | 18 Oct. 2018 |
| | | | Matrix: |
| Analytes | Units | RL | other |
| Alpha pinene | % | 0.001 | <0.001 |
| Beta pinene | % | 0.001 | <0.001 |
| Myrcene | % | 0.001 | <0.001 |
| Limonene | % | 0.001 | <0.001 |
| Terpinolene | % | 0.001 | <0.001 |
| Linalool | % | 0.001 | <0.001 |
| Terpineol | % | 0.001 | <0.001 |
| Caryophylene | % | 0.001 | <0.001 |
| Humulene | % | 0.001 | <0.001 |
| 3-carene | % | 0.001 | <0.001 |
| Cis-ocimene | % | 0.001 | <0.001 |
| Eucalyptol | % | 0.001 | <0.001 |
| Trans-ocimene | % | 0.001 | <0.001 |
| Fenchol | % | 0.001 | <0.001 |
| Bomeol | % | 0.001 | <0.001 |

Terpenes in cannabis oil

| | | | RPC Sample ID: |
|---|---|---|---|
| | | | 293340-6 |
| | | | Client Sample ID: |
| | | | J13 (Cannabis |
| | | | Extract in |
| | | | Hexanes) |
| | | | Date Sampled: |
| | | | 18 Oct. 2018 |
| | | | Matrix: |
| Analytes | Units | RL | other |
| Valencene | % | 0.001 | <0.001 |
| Cis-nerolidol | % | 0.001 | <0.001 |
| Trans-nerolidol | % | 0.001 | <0.001 |
| Guaiol | % | 0.001 | <0.001 |
| Alpha-bisabolol | % | 0.001 | <0.001 |
| Sabinene | % | 0.001 | <0.001 |
| Total terpenes | % | 0.001 | <0.001 |

This report relates only to the sample(s) and information provided to the laboratory.
Reported results are expressed on an as received basis.
RL = Reporting Limit, ND = Not Detected

### Trial 7—Collection of Cannabis Smoke Using Glass Fiber Filter Pads

In an ideal trapping system, the last impinger should have little or no detectable analyte in it. This is to ensure that analytes are not escaping the trapping system and that recoveries are maximized. In all impinger trials performed to this point, the last impinger contained measurable amounts of cannabinoids, which suggests that some analytes are escaping the system.

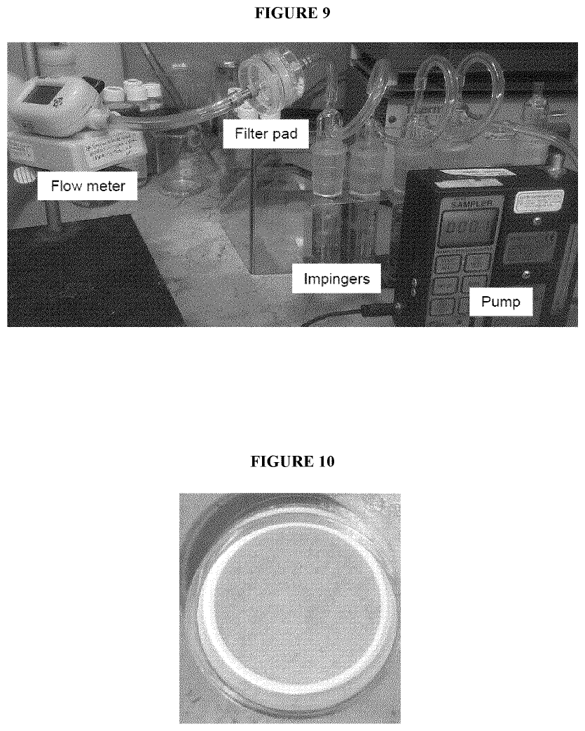

To address the concern of breakthrough, the use of high efficiency glass fiber filter pads was explored for trapping smoke particulate matter. An experiment was designed (Trial 7) in which the trapping system from Trial 5 (i.e. optimized impinger system) was modified to include a 37 mm high-efficiency glass fiber filter pad (pore size of 1 km) placed in series with the impingers. For cannabinoid collection, the trapping system consisted of a filter pad followed by 4 impingers, each containing 25 mL of methanol (FIG. 9). For terpene collection, the trapping system consisted of a filter pad followed by 2 impingers, each containing 10 mL of hexane.

Upon completion of smoke collection, the filter pads were extracted with either 25 mL of methanol (for cannabinoids) or 10 mL of hexane (for terpenes) followed by 20 minutes of sonication. Samples were then filtered with a 0.45 m Teflon filter prior to analysis.

The smoke collection parameters for this experiment are summarized in Table 25 and results are presented in Tables 26 and 27.

TABLE 25
Smoke Collection Parameters for Trial 7

| | |
|---|---|
| Mass of pellets | 226.9 mg (for cannabinoid trapping) |
| | 191.9 mg (for terpene trapping) |
| Flow rate through pipe | 0.56 L/min for cannabinoids |
| | 0.61 L/min for terpenes |
| Trapping system | 1 filter pad and 4 impingers |
| | for cannabinoid trapping |
| | 1 filter pad and 2 impingers for terpene trapping |
| Solvents/volumes | Methanol, 25 mL per impinger, |
| | for cannabinoid trapping Hexane, |
| | 10 mL per impinger, for terpene trapping |

TABLE 26
Cannabinoid Results for Trial 7

| | Mass recovered from | |
|---|---|---|
| Analyte | smoke (mg)* | Relative recovery (%)** |
| Total Della 9-THC (THC + THCA) | 8.73 | 44 |
| Total CBD (CBD + CBDA) | 2.25 | 38 |
| Total CBG (CBG + CBGA) | 0.33 | 39 |
| CBN | 0.80 | (235) |
| CBC | 0.25 | (357) |

*See Certificate of Analysis 293753-OAS (Appendix G).
**Calculated using unburnt pellet extract results as a benchmark.

TABLE 27
Terpene Results for Trial 7

| | Mass recovered from | |
|---|---|---|
| Analyte | smoke (mg)* | Relative recovery (%)** |
| Total Terpenes | 0.08 | 17 |
| Terpineol | 0.02 | 52 |
| Caryophyllene | 0.02 | 10 |
| Trans-nerolidol | 0.01 | n/a |
| Alpha-bisabolol | 0.03 | 78 |

*See Certificate of Analysis 293753-OAS (Appendix G).
**Calculated using unburnt pellet extract results as a benchmark.

The filter pad effectively trapped cannabinoids and terpenes: 99.8% of cannabinoids and 100% of terpenes recovered were trapped on the filter pads. Very little or no analyte was detected in the impingers connected to the filter pad. This data supports the thought that the majority of target analytes are indeed in the particulate fraction and can easily be captured by employing the glass filter.

The smoke particulate matter trapped by the filter pad can be seen in FIG. 10. The filter pad, originally a white color, turned a dark yellow after the smoke collection was complete. Compared to the impinger trapping system (Trial 5), the relative recoveries of THC, CBD, and CBG were very similar, differing only by a few percent. Although the relative recovery of total terpenes was similar to that of Trials 1 and 2, the number of reported terpenes was more comprehensive and included terpineol, caryophyllene, trans-nerolidol, and alpha-bisabolol.

Given the similar performance of the impinger trapping system for cannabinoids and terpenes to the filter pad trap, both approaches were evaluated in more detail for vapor collection efficiency (see Example 5).

### Example 5—Trapping Vapor from Cannabis Pellets Heated in a Vaporizer Using Methods Developed in Example 4

Analytical methods developed to trap smoke (Example 4) were applied to trapping the vapor produced from cannabis pellets (PPP001) heated in the Mighty vaporizer (FIG. 11A). Unlike the Raydiator pipe, the vaporizer does not combust the pellet, but rather heats the material at a set temperature. Temperatures on the vaporizer can be set between 40° C. and 210° C. Cannabis pellets can be placed directly into the heating chamber of the Might Medic® vaporizer (FIG. 11B) or loaded into metal capsules that can be inserted into the heating chamber/receptacle (FIG. 11C).

Prior to performing vapor trapping experiments, the stability of the heating chamber was evaluated to ensure that a continuous stream of air drawn through the heating chamber would not cool the chamber. It was found that even at high draw rates (up to 2 L/min), the vaporizer temperature was maintained within 3° C. of the set temperature of 195° C. (i.e. 195±3° C.).

Cannabinoids in oil by HPLC

| | | | RPC Sample ID: | | | | | |
|---|---|---|---|---|---|---|---|---|
| | | | 293753-04 | 293753-05 | 293753-06 | 293753-07 | 293753-08 | 293753-12 |
| | | | Client Sample ID: | | | | | |
| | | | N11 | N12 | N13 | N14 | N15 | P11 |
| | | | (Cannabis | (Cannabis | (Cannabis | (Cannabis | (Cannabis | (Cannabis |
| | | | Vapor In | Vapor In | Vapor In | Vapor In | Vapor In | Vapor In |
| | | | Methanol) | Methanol) | Methanol) | Methanol) | Methanol) | Methanol) |
| | | | Date Sampled: | | | | | |
| | | | 22 Oct. 2018 | 22 Oct. 2018 | 22 Oct. 2018 | 22 Oct. 2018 | 22 Oct. 2018 | 22 Oct. 2018 |
| | | | Matrix: | | | | | |
| Analytes | Units | RL | other | other | other | other | other | other |
| Delta 9-Tetrahydrocannabinol | mg/ml | 0.001 | 0.310 | ND | ND | ND | <0.001 | 0.347 |
| (Delta 9-THC) | | | | | | | | |
| Delta 9-Tetrahydrocannabinolic | mg/ml | 0.001 | ND | <0.001 | ND | ND | ND | 0.002 |
| Acid (Delta 9-THCA) | | | | | | | | |
| Cannabidiol (CBD) | mg/ml | 0.001 | 0.123 | ND | ND | ND | ND | 0.088 |
| Cannabidiolic Acid (CBDA) | mg/ml | 0.001 | 0.003 | ND | ND | ND | ND | 0.002 |
| Cannabigerol (CBG) | mg/ml | 0.001 | 0.010 | ND | ND | ND | ND | 0.012 |
| Cannabigerolic Acid (CBGA) | mg/ml | 0.001 | 0.001 | <0.001 | ND | ND | ND | <0.001 |
| Cannabinol (CBN) | mg/ml | 0.001 | 0.057 | ND | ND | ND | ND | 0.032 |
| Cannabichromene (CBC) | mg/ml | 0.001 | 0.009 | ND | ND | ND | ND | 0.010 |
| Total Delta 9-Tetrahydrocannabinol | mg/ml | 0.001 | 0.310 | <0.001 | ND | ND | <0.001 | 0.349 |
| (Delta 9-THC) | | | | | | | | |
| Total Cannabidiol (CBD) | mg/ml | 0.001 | 0.126 | ND | ND | ND | ND | 0.090 |
| Total Cannabigerol (CBG) | mg/ml | 0.001 | 0.011 | <0.001 | ND | ND | ND | 0.012 |

This report relates only to the sample(s) and information provided to the laboratory.
Reported results are expressed on an as received basis.
RL = Reporting Limit,
ND = Not Detected

Cannabinoids in oil by HPLC

| | | | RPC Sample ID: | | | |
|---|---|---|---|---|---|---|
| | | | 293753-13 | 293753-14 | 293753-15 | 293753-16 |
| | | | Client Sample ID: | | | |
| | | | P12 | P13 | P14 | P15 |
| | | | (Cannabis Vapor | (Cannabis Vapor | (Cannabis Vapor | (Cannabis Vapor |
| | | | In Methanol) | In Methanol) | In Methanol) | In Methanol) |
| | | | Date Sampled: | | | |
| | | | 22 Oct. 2018 | 22 Oct. 2018 | 22 Oct. 2018 | 22 Oct. 2018 |
| | | | Matrix: | | | |
| Analytes | Units | RL | other | other | other | other |
| Delta 9-Tetrahydrocannabinol (Delta 9-THC) | mg/ml | 0.001 | ND | ND | ND | ND |
| Delta 9-Tetrahydrocannabinolic Acid (Delta 9-THCA) | mg/ml | 0.001 | ND | ND | ND | ND |
| Cannabidiol (CBD) | mg/ml | 0.001 | ND | ND | ND | ND |
| Cannabidiolic Acid (CBDA) | mg/ml | 0.001 | ND | ND | ND | ND |
| Cannabigerol (CBG) | mg/ml | 0.001 | ND | ND | ND | ND |
| Cannabigerolic Acid (CBGA) | mg/ml | 0.001 | 0.001 | ND | ND | ND |
| Cannabinol (CBN) | mg/ml | 0.001 | ND | ND | ND | ND |
| Cannabichromene (CBC) | mg/ml | 0.001 | ND | ND | ND | ND |
| Total Delta 9-Tetrahydrocannabinol (Delta 9-THC) | mg/ml | 0.001 | ND | ND | ND | ND |
| Total Cannabidiol (CBD) | mg/ml | 0.001 | ND | ND | ND | ND |
| Total Cannabigerol (CBG) | mg/ml | 0.001 | 0.001 | ND | ND | ND |

This report relates only to the sample(s) and information provided to the laboratory.
Reported results are expressed on an as received basis.
RL = Reporting Limit,
ND = Not Detected

Terpenes in cannabis oil

| | | | RPC Sample ID: | | | | | |
|---|---|---|---|---|---|---|---|---|
| | | | 293753-01 | 293753-02 | 293753-03 | 293753-09 | 293753-10 | 293753-11 |
| | | | Client Sample ID: | | | | | |
| | | | N01 | N02 | N03 | P01 | P02 | P03 |
| | | | (Cannabis | (Cannabis | (Cannabis | (Cannabis | (Cannabis | (Cannabis |
| | | | Vapor In | Vapor In | Vapor In | Vapor In | Vapor In | Vapor In |
| | | | Hexane) | Hexane) | Hexane) | Hexane) | Hexane) | Hexane) |
| | | | Date Sampled: | | | | | |
| | | | 22 Oct. 2018 | 22 Oct. 2018 | 22 Oct. 2018 | 22 Oct. 2018 | 22 Oct. 2018 | 22 Oct. 2018 |
| | | | Matrix: | | | | | |
| Analytes | Units | RL | other | other | other | other | other | other |
| Alpha pinene | mg/mL | 0.001 | <0.001 | <0.001 | <0.001 | <0.001 | <0.001 | <0.001 |
| Beta pinene | mg/mL | 0.001 | <0.001 | <0.001 | <0.001 | <0.001 | <0.001 | <0.001 |
| Myrcene | mg/mL | 0.001 | <0.001 | <0.001 | <0.001 | <0.001 | <0.001 | <0.001 |
| Limonene | mg/mL | 0.001 | <0.001 | <0.001 | <0.001 | <0.001 | <0.001 | <0.001 |
| Terpinolene | mg/mL | 0.001 | <0.001 | <0.001 | <0.001 | <0.001 | <0.001 | <0.001 |
| Linalool | mg/mL | 0.001 | 0.001 | <0.001 | <0.001 | <0.001 | <0.001 | <0.001 |
| Terpineol | mg/mL | 0.001 | 0.003 | <0.001 | <0.001 | 0.002 | <0.001 | <0.001 |
| Caryophylene | mg/mL | 0.001 | 0.003 | <0.001 | <0.001 | 0.002 | <0.001 | <0.001 |
| Humulene | mg/mL | 0.001 | 0.001 | <0.001 | <0.001 | <0.001 | <0.001 | <0.001 |
| 3-carene | mg/mL | 0.001 | <0.001 | <0.001 | <0.001 | <0.001 | <0.001 | <0.001 |
| Cis-ocimene | mg/mL | 0.001 | <0.001 | <0.001 | <0.001 | <0.001 | <0.001 | <0.001 |
| Eucalyptol | mg/mL | 0.001 | <0.001 | <0.001 | <0.001 | <0.001 | <0.001 | <0.001 |
| Trans-ocimene | mg/mL | 0.001 | <0.001 | <0.001 | <0.001 | <0.001 | <0.001 | <0.001 |
| Fenchol | mg/mL | 0.001 | <0.001 | <0.001 | <0.001 | <0.001 | <0.001 | <0.001 |
| Borneol | mg/mL | 0.001 | <0.001 | <0.001 | <0.001 | <0.001 | <0.001 | <0.001 |
| Valencene | mg/mL | 0.001 | 0.001 | <0.001 | <0.001 | <0.001 | <0.001 | <0.001 |
| Cis-nerolidol | mg/mL | 0.001 | <0.001 | <0.001 | <0.001 | <0.001 | <0.001 | <0.001 |
| Trans-nerolidol | mg/mL | 0.001 | 0.002 | <0.001 | <0.001 | 0.001 | <0.001 | <0.001 |
| Gualol | mg/mL | 0.001 | <0.001 | <0.001 | <0.001 | <0.001 | <0.001 | <0.001 |
| Alpha-bisabolol | mg/mL | 0.001 | 0.005 | <0.001 | <0.001 | 0.003 | <0.001 | <0.001 |
| Sabinene | mg/mL | 0.001 | <0.001 | <0.001 | <0.001 | <0.001 | <0.001 | <0.001 |
| Total terpenes | mg/mL | 0.001 | 0.016 | <0.001 | <0.001 | 0.008 | <0.001 | <0.001 |

This report relates only to the sample(s) and information provided to the laboratory.
Reported results are expressed on an as received basis.
RL = Reporting Limit,
ND = Not Detected

### Trial 8—Collection of Cannabis Vapor at 195° C. From Broken Pellets Using Impingers

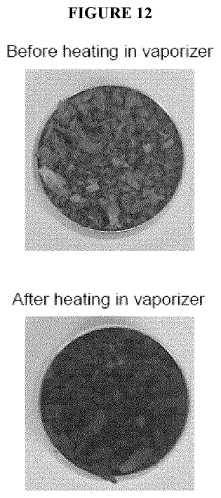

The first vapor trapping experiment (Trial 8) involved the use of fragmented pellet material loaded into capsules. Unlike the Raydiator pipe, where smoke collection ended when all the material had burned, it was more challenging to determine when the pellet material was completely vaporized. For that reason, in order to maintain consistency between vapor collections, all experiments were conducted for six minutes. Specifically, the vaporizer was first turned on and allowed to reach the set temperature of 195° C. at which point the pump was turned on and allowed to draw vapor through the impinger system for six minutes. After six minutes, it was observed that all the pellet material within the capsule had transformed from a green to a brown color (FIG. 12). The vapor collection parameters employed for this set of experiments are shown in Table 28 and the results are given in Tables 29 and 30.

TABLE 28
Vapor Collection Parameters for Trial 8

| Mass of pellets | 258.6 mg (for cannabinoid trapping) | |
|---|---|---|
| (broken pellets) | 258.2 mg (for terpene trapping) | |
| Flow rate through | 0.53 L/min for cannabinoids | |
| vaporizer | 0.50 L/min for terpenes | |
| Trapping system | 5 impingers for cannabinoid trapping | |
| | 3 impingers for terpene trapping | |
| Solvents/volumes | Methanol, 25 mL per impinger, | |
| | for cannabinoid trapping Hexane, | |
| | 10 mL per impinger, for terpene trapping | |
| Vaporizer temperature | 195° | C. |
| Collection time | 6 | minutes |

TABLE 29
Cannabinoid Results for Trial 8

| | Mass recovered from | |
|---|---|---|
| Analyte | vapor (mg)* | Relative recovery (%)** |
| Total Delta 9-THC (THC + THCA) | 5.20 | 23 |
| Total CBD (CBD + CBDA) | 2.10 | 31 |
| Total CBG (CBG + CBGA) | 0.13 | 13 |
| CBN | 0.68 | (174) |
| CBC | 0.15 | (83) |

*See Certificate of Analysis 293615-OAS (Appendix H).
**Calculated using unburnt pellet extract results as a benchmark.

TABLE 30
Terpene Results for Trial 8

| | Mass recovered from | |
|---|---|---|
| Analyte | vapor (mg)* | Relative recovery (%)** |
| Total Terpenes | 0.10 | 15 |
| Linalool | 0.02 | 26 |
| Terpineol | 0.02 | 39 |
| Caryophyllene | 0.04 | 15 |
| Fenchol | 0.01 | 39 |
| Alpha-bisabolol | 0.01 | 19 |

*See Certificate of Analysis 293615-OAS (Appendix H).
**Calculated using unburnt pellet extract results as a benchmark.

Overall, THC, CBD, and CBG recoveries for cannabis vaporized at 195° C. were lower than those observed for the smoked cannabis in the Raydiator pipe. This result is consistent with literature reports comparing smoked and vaporized cannabis [3]. For example, Pomahacova et al. found that the THC recovered from vapor generated at 200° C. was about half ofthat recovered from smoked cannabis cigarettes. Interestingly, at a slightly higher vaporization temperature of 230° C., the researchers observed that THC recoveries from vapor exceeded those observed from the cigarette smoke by roughly 56%. This suggests that in order to improve cannabinoid recoveries from pellets heated in the Mighty vaporizer, the temperature should be increased. Almost all the cannabinoids (i.e. 99.7%) were detected in their neutral, decarboxylated forms, which indicates that heating pellets in a vaporizer may be an efficient way of ensuring decarboxylation. In contrast, smoked cannabis is more likely to result in particulate matter being inhaled, which can contain unconverted cannabinoid acids, as evidenced by Trial 1. This suggests that smoking is a possibly less efficient method for conversion of cannabinoid acids to their neutral forms (i.e. conversion ofTHCA to THC, CBDA to CBD).

Terpenes, which are significantly more volatile than cannabinoids, yielded recoveries for the Mighty that were comparable to those found in the Raydiator smoke from Trials 1, 2, and 7. Terpenes detected included linalool, terpineol, caryophyllene, fenchol, and alpha-bisabolol, all of which were also present in the dry pellet extract (Table 9).

Cannabinoids in oil by HPLC

| | | | RPC Sample ID: | | | | | |
|---|---|---|---|---|---|---|---|---|
| | | | 293615-04 | 293615-05 | 293615-06 | 293615-07 | 293615-08 | 293615-12 |
| | | | Client Sample ID: | | | | | |
| | | | | | | | | M11 |
| | | | | | | | | (Cannabis |
| | | | L11 | L12 | L13 | L14 | L15 | Vapor In |
| | | | (Cannabis | (Cannabis | (Cannabis | (Cannabis | (Cannabis | Methanol) |
| | | | Vapor In | Vapor In | Vapor In | Vapor In | Vapor In | Date Sampled: |
| | | | Methanol) | Methanol) | Methanol) | Methanol) | Methanol) | 19 Oct. 2018 |
| | | | Matrix: | | | | | |
| Analytes | Units | RL | other | other | other | other | other | other |
| Delta 9-Tetrahydrocannabinol | mg/ml | 0.001 | 0.013 | 0.016 | 0.025 | 0.011 | 0.007 | 0.049 |
| (Delta 9-THC) | | | | | | | | |
| Delta 9-Tetrahydrocannabinolic | mg/ml | 0.001 | ND | ND | ND | ND | ND | ND |
| Acid (Delta 9-THCA) | | | | | | | | |
| Cannabidiol (CBD) | mg/ml | 0.001 | 0.005 | 0.007 | 0.011 | 0.005 | 0.003 | 0.020 |
| Cannabidiolic Acid (CBDA) | mg/ml | 0.001 | ND | ND | ND | ND | ND | ND |
| Cannabigerol (CBG) | mg/ml | 0.001 | ND | ND | ND | ND | ND | 0.001 |
| Cannabigerolic Acid (CBGA) | mg/ml | 0.001 | ND | ND | ND | ND | ND | 0.001 |
| Cannabinol (CBN) | mg/ml | 0.001 | 0.001 | 0.002 | 0.003 | 0.001 | 0.001 | 0.006 |
| Cannabichromene (CBC) | mg/ml | 0.001 | ND | ND | 0.001 | ND | ND | 0.002 |
| Total Delta 9-Tetrahydrocannabinol | mg/ml | 0.001 | 0.013 | 0.016 | 0.025 | 0.011 | 0.007 | 0.049 |
| (Della 9-THC) | | | | | | | | |
| Total Cannabidiol (CBD) | mg/ml | 0.001 | 0.005 | 0.007 | 0.011 | 0.005 | 0.003 | 0.020 |
| Total Cannabigerol (CBG) | mg/ml | 0.001 | ND | ND | ND | ND | ND | 0.001 |

This report relates only to the sample(s) and information provided to the laboratory.
Reported results are expressed on an as received basis.
RL = Reporting Limit,
ND = Not Detected

Cannabinoids in oil by HPLC

| | | | RPC Sample ID: | | | |
|---|---|---|---|---|---|---|
| | | | 293615-13 | 293615-14 | 293615-15 | 293615-16 |
| | | | Client Sample ID: | | | |
| | | | M12 | M13 | M14 | M15 |
| | | | (Cannabis Vapor | (Cannabis Vapor | (Cannabis Vapor | (Cannabis Vapor |
| | | | In Methanol) | In Methanol) | In Methanol) | In Methanol) |
| | | | Date Sampled: | | | |
| | | | 19 Oct. 2018 | 19 Oct. 2018 | 19 Oct. 2018 | 19 Oct. 2018 |
| | | | Matrix: | | | |
| Analytes | Units | RL | other | other | other | other |
| Delta 9-Tetrahydrocannabinol (Delta 9-THC) | mg/ml | 0.001 | 0.071 | 0.063 | 0.017 | 0.008 |
| Delta 9-Tetrahydrocannabinolic Acid (Delta 9-THCA) | mg/ml | 0.001 | ND | ND | ND | ND |
| Cannabidiol (CBD) | mg/ml | 0.001 | 0.029 | 0.025 | 0.007 | 0.003 |
| Cannabidiolic Acid (CBDA) | mg/ml | 0.001 | ND | ND | ND | ND |
| Cannabigerol (CBG) | mg/ml | 0.001 | 0.002 | 0.002 | ND | ND |
| Cannabigerolic Acid (CBGA) | mg/ml | 0.001 | ND | ND | ND | ND |
| Cannabinol (CBN) | mg/ml | 0.001 | 0.009 | 0.009 | 0.002 | 0.001 |
| Cannabichromene (CBC) | mg/ml | 0.001 | 0.002 | 0.002 | ND | ND |
| Total Delta 9-Tetrahydrocannabinol (Delta 9-THC) | mg/ml | 0.001 | 0.071 | 0.063 | 0.017 | 0.008 |
| Total Cannabidiol (CBD) | mg/ml | 0.001 | 0.029 | 0.025 | 0.007 | 0.003 |
| Total Cannabigerol (CBG) | mg/ml | 0.001 | 0.002 | 0.002 | ND | ND |

This report relates only to the sample(s) and information provided to the laboratory.
Reported results are expressed on an as received basis.
RL = Reporting Limit,
ND = Not Detected

Terpenes in cannabis oil

| | | | RPC Sample ID: | | | | | |
|---|---|---|---|---|---|---|---|---|
| | | | 293615-01 | 293615-02 | 293615-03 | 293615-09 | 293615-10 | 293615-11 |
| | | | Client Sample ID: | | | | | |
| | | | | | | M01 | M02 | M03 |
| | | | | | | (Cannabis | (Cannabis | (Cannabis |
| | | | L01 | L02 | L03 | Vapor In | Vapor In | Vapor In |
| | | | (Cannabis | (Cannabis | (Cannabis | Hexane) | Hexane) | Hexane) |
| | | | Vapor In | Vapor In | Vapor In | Date Sampled: | | |
| | | | Hexane) | Hexane) | Hexane) | 19 Oct. 2018 | 19 Oct. 2018 | 19 Oct. 2018 |
| | | | Matrix: | | | | | |
| Analytes | Units | RL | other | other | other | other | other | other |
| Alpha pinene | mg/mL | 0.001 | <0.001 | <0.001 | <0.001 | <0.001 | <0.001 | <0.001 |
| Beta pinene | mg/mL | 0.001 | <0.001 | <0.001 | <0.001 | <0.001 | <0.001 | <0.001 |
| Myrcene | mg/mL | 0.001 | <0.001 | <0.001 | <0.001 | <0.001 | <0.001 | <0.001 |
| Limonene | mg/mL | 0.001 | <0.001 | <0.001 | <0.001 | <0.001 | <0.001 | <0.001 |
| Terpinolene | mg/mL | 0.001 | <0.001 | <0.001 | <0.001 | <0.001 | <0.001 | <0.001 |
| Linalool | mg/mL | 0.001 | 0.003 | <0.001 | <0.001 | 0.002 | <0.001 | <0.001 |
| Terpineol | mg/mL | 0.001 | 0.002 | <0.001 | <0.001 | 0.002 | <0.001 | <0.001 |
| Caryophylene | mg/mL | 0.001 | 0.004 | 0.001 | <0.001 | 0.003 | 0.001 | <0.001 |
| Humulene | mg/mL | 0.001 | 0.001 | <0.001 | <0.001 | <0.001 | <0.001 | <0.001 |
| 3-carene | mg/mL | 0.001 | <0.001 | <0.001 | <0.001 | <0.001 | <0.001 | <0.001 |
| Cis-ocimene | mg/mL | 0.001 | <0.001 | <0.001 | <0.001 | <0.001 | <0.001 | <0.001 |
| Eucalyptol | mg/mL | 0.001 | <0.001 | <0.001 | <0.001 | <0.001 | <0.001 | <0.001 |
| Trans-ocimene | mg/mL | 0.001 | <0.001 | <0.001 | <0.001 | <0.001 | <0.001 | <0.001 |
| Fenchol | mg/mL | 0.001 | 0.002 | <0.001 | <0.001 | 0.001 | <0.001 | <0.001 |
| Bomneol | mg/mL | 0.001 | <0.001 | <0.001 | <0.001 | <0.001 | <0.001 | <0.001 |
| Valencene | mg/mL | 0.001 | <0.001 | <0.001 | <0.001 | <0.001 | <0.001 | <0.001 |
| Cis-nerolidol | mg/mL | 0.001 | <0.001 | <0.001 | <0.001 | <0.001 | <0.001 | <0.001 |
| Trans-nerolidol | mg/mL | 0.001 | <0.001 | <0.001 | <0.001 | <0.001 | <0.001 | <0.001 |
| Guaiol | mg/mL | 0.001 | <0.001 | <0.001 | <0.001 | <0.001 | <0.001 | <0.001 |
| Alpha-bisabolol | mg/mL | 0.001 | <0.001 | <0.001 | <0.001 | <0.001 | 0.001 | 0.001 |
| Sabinene | mg/mL | 0.001 | <0.001 | <0.001 | <0.001 | <0.001 | <0.001 | <0.001 |
| Total terpenes | mg/mL | 0.001 | 0.012 | 0.001 | <0.001 | 0.008 | 0.002 | 0.001 |

This report relates only to the sample(s) and information provided to the laboratory.
Reported results are expressed on an as received basis.
RL = Reporting Limit,
ND = Not Detected

### Trial 9—Collection of Cannabis Vapor at 195° C. From Intact Pellets Using Impingers

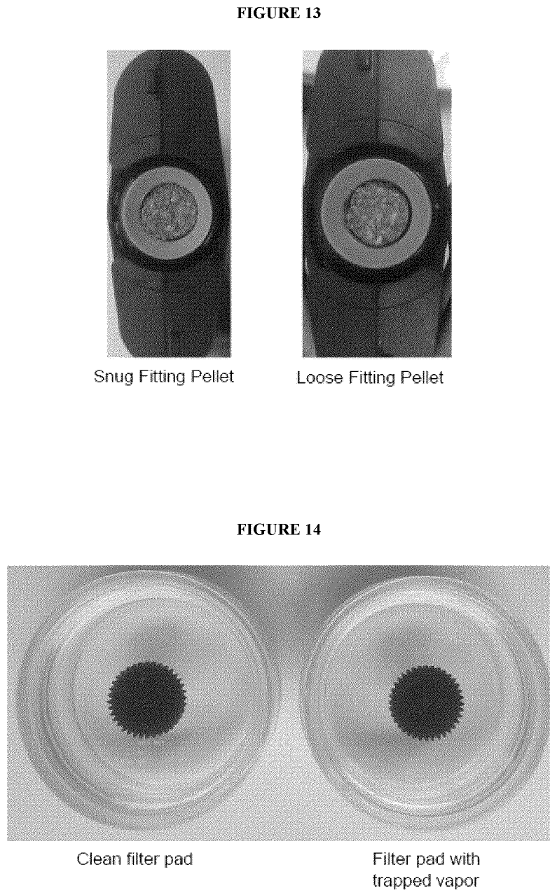

A subsequent experiment (Trial 9) was conducted in order to investigate the effect of using an intact pellet (no capsule) on cannabinoid and terpene recovery using the same collection parameters used in Trial 8. When loading intact pellets into the heating chamber of the vaporizer, it was found that differences in pellet uniformity (i.e. differences in mass and diameter between pellets) affected how pellets fit into the heating chamber (FIG. 13). Larger intact pellets with masses exceeding 300 mg had to be pressed into the heating chamber and fit snuggly with no space between the pellet and the chamber walls. In contrast, smaller intact pellets with masses of roughly 280 mg could easily be placed into the heating chamber, with space around the edges still remaining to better accommodate air flow. Only intact pellets that fit easily into the vaporizer heating chamber were used for Trial 9 experiments. For this set of experiments, vapor collection parameters are summarized in Table 31 and results are given in Tables 32 and 33.

TABLE 31
Vapor Collection Parameters for Trial 9

| Mass of pellets | 256.0 mg (for cannabinoid trapping) | |
|---|---|---|
| (intact pellets) | 268.0 mg (for terpene trapping) | |
| Flow rate through | 0.58 L/min for cannabinoids | |
| vaporizer | 0.59 L/min for terpenes | |
| Trapping system | 5 impingers for cannabinoid trapping | |
| | 3 impingers for terpene trapping | |
| Solvents/volumes | Methanol, 25 mL per impinger, | |
| | for cannabinoid trapping Hexane, | |
| | 10 mL per impinger, for terpene trapping | |
| Vaporizer temperature | 195° | C. |
| Collection time | 6 | minutes |

TABLE 32
Cannabinoid Results for Trial 9

| | Mass recovered from | |
|---|---|---|
| Analyte | vapor (mg)* | Relative recovery (%)** |
| Total Delta 9-THC (THC + THCA) | 1.80 | 8 |
| Total CBD (CBD + CBDA) | 0.78 | 12 |
| Total CBG (CBG + CBGA) | 0.00 | 0 |
| CBN | 0.20 | (52) |
| CBC | 0.03 | (14) |

*See Certificate of Analysis 293615-OAS (Appendix I).
**Calculated using unburnt pellet extract results as a benchmark.

TABLE 33
Terpene Results for Trial 9

| | Mass recovered from | |
|---|---|---|
| Analyte | vapor (mg)* | Relative recovery (%)** |
| Total Terpenes | 0.13 | 19 |
| Linalool | 0.03 | 37 |
| Terpineol | 0.02 | 37 |
| Caryophyllene | 0.05 | 19 |
| Humulene | 0.01 | 9 |
| Fenchol | 0.02 | 75 |

*See Certificate of Analysis 293615-OAS (Appendix I).
**Calculated using unburnt pellet extract results as a benchmark.

Cannabinoid recoveries from the vapor of intact pellets were significantly lower than that of the fragmented (loose) material loaded into the capsules. These initial findings suggest that vaporization efficiency of cannabinoids is reduced in a more compressed pellet. Additional experiments, in which a larger number of intact pellets are evaluated should be conducted to better understand the recoveries of cannabinoids from intact pellets.

Increasing the temperature of the vaporizer to help improve recoveries should also be explored. Although cannabinoid recoveries were lower than those observed in Trial 8, all cannabinoids detected were decarboxylated (i.e. no acidic forms).

Interestingly, unlike cannabinoids, the extraction efficiency of terpene analytes was not affected by using an intact pellet. This is likely due to the much more volatile nature of terpene molecules relative to cannabinoids. Terpenes detected in this set of experiments included linalool, terpineol, caryophyllene, humulene, and fenchol.

Cannabinoids in oil by HPLC

| | | | RPC Sample ID: | | | | | |
|---|---|---|---|---|---|---|---|---|
| | | | 293615-04 | 293615-05 | 293615-06 | 293615-07 | 293615-08 | 293615-12 |
| | | | Client Sample ID: | | | | | |
| | | | | | | | | M11 |
| | | | | | | | | (Cannabis |
| | | | L11 | L12 | L13 | L14 | L15 | Vapor In |
| | | | (Cannabis | (Cannabis | (Cannabis | (Cannabis | (Cannabis | Methanol) |
| | | | Vapor In | Vapor In | Vapor In | Vapor In | Vapor In | Date Sampled: |
| | | | Methanol) | Methanol) | Methanol) | Methanol) | Methanol) | 19 Oct. 2018 |
| | | | Matrix: | | | | | |
| Analytes | Units | RL | other | other | other | other | other | other |
| Delta 9-Tetrahydrocannabinol | mg/ml | 0.001 | 0.013 | 0.016 | 0.025 | 0.011 | 0.007 | 0.049 |
| (Delta 9-THC) | | | | | | | | |
| Delta 9-Tetrahydrocannabinolic | mg/ml | 0.001 | ND | ND | ND | ND | ND | ND |
| Acid (Delta 9-THCA) | | | | | | | | |
| Cannabidiol (CBD) | mg/ml | 0.001 | 0.005 | 0.007 | 0.011 | 0.005 | 0.003 | 0.020 |
| Cannabidiolic Acid (CBDA) | mg/ml | 0.001 | ND | ND | ND | ND | ND | ND |
| Cannabigerol (CBG) | mg/ml | 0.001 | ND | ND | ND | ND | ND | 0.001 |
| Cannabigerolic Acid (CBGA) | mg/ml | 0.001 | ND | ND | ND | ND | ND | 0.001 |
| Cannabinol (CBN) | mg/ml | 0.001 | 0.001 | 0.002 | 0.003 | 0.001 | 0.001 | 0.006 |
| Cannabichromene (CBC) | mg/ml | 0.001 | ND | ND | 0.001 | ND | ND | 0.002 |
| Total Delta 9-Tetrahydrocannabinol | mg/ml | 0.001 | 0.013 | 0.016 | 0.025 | 0.011 | 0.007 | 0.049 |
| (Delta 9-THC) | | | | | | | | |
| Total Cannabidiol (CBD) | mg/ml | 0.001 | 0.005 | 0.007 | 0.011 | 0.005 | 0.003 | 0.020 |
| Total Cannabigerol (CBG) | mg/ml | 0.001 | ND | ND | ND | ND | ND | 0.001 |

This report relates only to the sample(s) and information provided to the laboratory.
Reported results are expressed on an as received basis.
RL = Reporting Limit,
ND = Not Detected

Cannabinoids in oil by HPLC

| | | | RPC Sample ID: | | | |
|---|---|---|---|---|---|---|
| | | | 293615-13 | 293615-14 | 293615-15 | 293615-16 |
| | | | Client Sample ID: | | | |
| | | | M12 | M13 | M14 | M15 |
| | | | (Cannabis Vapor | (Cannabis Vapor | (Cannabis Vapor | (Cannabis Vapor |
| | | | In Methanol) | In Methanol) | In Methanol) | In Methanol) |
| | | | Date Sampled: | | | |
| | | | 19 Oct. 2018 | 19 Oct. 2018 | 19 Oct. 2018 | 19 Oct. 2018 |
| | | | Matrix: | | | |
| Analytes | Units | RL | other | other | other | other |
| Delta 9-Tetrahydrocannabinol (Delta 9-THC) | mg/ml | 0.001 | 0.071 | 0.063 | 0.017 | 0.008 |
| Delta 9-Tetrahydrocannabinolic Acid (Delta 9-THCA) | mg/ml | 0.001 | ND | ND | ND | ND |
| Cannabidiol (CBD) | mg/ml | 0.001 | 0.029 | 0.025 | 0.007 | 0.003 |
| Cannabidiolic Acid (CBDA) | mg/ml | 0.001 | ND | ND | ND | ND |
| Cannabigerol (CBG) | mg/ml | 0.001 | 0.002 | 0.002 | ND | ND |
| Cannabigerolic Acid (CBGA) | mg/ml | 0.001 | ND | ND | ND | ND |
| Cannabinol (CBN) | mg/ml | 0.001 | 0.009 | 0.009 | 0.002 | 0.001 |
| Cannabichromene (CBC) | mg/ml | 0.001 | 0.002 | 0.002 | ND | ND |
| Total Delta 9-Tetrahydrocannabinol (Delta 9-THC) | mg/ml | 0.001 | 0.071 | 0.063 | 0.017 | 0.008 |
| Total Cannabidiol (CBD) | mg/ml | 0.001 | 0.029 | 0.025 | 0.007 | 0.003 |
| Total Cannabigerol (CBG) | mg/ml | 0.001 | 0.002 | 0.002 | ND | ND |

This report relates only to the sample(s) and information provided to the laboratory.
Reported results are expressed on an as received basis.
RL = Reporting Limit,
ND = Not Detected

Terpenes in cannabis oil

| | | | RPC Sample ID: | | | | | |
|---|---|---|---|---|---|---|---|---|
| | | | 293615-01 | 293615-02 | 293615-03 | 293615-09 | 293615-10 | 293615-11 |
| | | | Client Sample ID: | | | | | |
| | | | | | | M01 | M02 | M03 |
| | | | | | | (Cannabis | (Cannabis | (Cannabis |
| | | | L01 | L02 | L03 | Vapor In | Vapor In | Vapor In |
| | | | (Cannabis | (Cannabis | (Cannabis | Hexane) | Hexane) | Hexane) |
| | | | Vapor In | Vapor In | Vapor In | Date Sampled: | | |
| | | | Hexane) | Hexane) | Hexane) | 19 Oct. 2018 | 19 Oct. 2018 | 19 Oct. 2018 |
| | | | Matrix: | | | | | |
| Analytes | Units | RL | other | other | other | other | other | other |
| Alpha pinene | mg/mL | 0.001 | <0.001 | <0.001 | <0.001 | <0.001 | <0.001 | <0.001 |
| Beta pinene | mg/mL | 0.001 | <0.001 | <0.001 | <0.001 | <0.001 | <0.001 | <0.001 |
| Myrcene | mg/mL | 0.001 | <0.001 | <0.001 | <0.001 | <0.001 | <0.001 | <0.001 |
| Limonene | mg/mL | 0.001 | <0.001 | <0.001 | <0.001 | <0.001 | <0.001 | <0.001 |
| Terpinolene | mg/mL | 0.001 | <0.001 | <0.001 | <0.001 | <0.001 | <0.001 | <0.001 |
| Linalool | mg/mL | 0.001 | 0.003 | <0.001 | <0.001 | 0.002 | <0.001 | <0.001 |
| Terpineol | mg/mL | 0.001 | 0.002 | <0.001 | <0.001 | 0.002 | <0.001 | <0.001 |
| Caryophylene | mg/mL | 0.001 | 0.004 | 0.001 | <0.001 | 0.003 | 0.001 | <0.001 |
| Humulene | mg/mL | 0.001 | 0.001 | <0.001 | <0.001 | <0.001 | <0.001 | <0.001 |
| 3-carene | mg/mL | 0.001 | <0.001 | <0.001 | <0.001 | <0.001 | <0.001 | <0.001 |
| Cis-ocimene | mg/mL | 0.001 | <0.001 | <0.001 | <0.001 | <0.001 | <0.001 | <0.001 |
| Eucalyptol | mg/mL | 0.001 | <0.001 | <0.001 | <0.001 | <0.001 | <0.001 | <0.001 |
| Trans-ocimene | mg/mL | 0.001 | <0.001 | <0.001 | <0.001 | <0.001 | <0.001 | <0.001 |
| Fenchol | mg/mL | 0.001 | 0.002 | <0.001 | <0.001 | 0.001 | <0.001 | <0.001 |
| Borneol | mg/mL | 0.001 | <0.001 | <0.001 | <0.001 | <0.001 | <0.001 | <0.001 |
| Valencene | mg/mL | 0.001 | <0.001 | <0.001 | <0.001 | <0.001 | <0.001 | <0.001 |
| Cis-nerolidol | mg/mL | 0.001 | <0.001 | <0.001 | <0.001 | <0.001 | <0.001 | <0.001 |
| Trans-nerolidol | mg/mL | 0.001 | <0.001 | <0.001 | <0.001 | <0.001 | <0.001 | <0.001 |
| Guaiol | mg/mL | 0.001 | <0.001 | <0.001 | <0.001 | <0.001 | <0.001 | <0.001 |
| Alpha-bisabolol | mg/mL | 0.001 | <0.001 | <0.001 | <0.001 | <0.001 | 0.001 | 0.001 |
| Sabinene | mg/mL | 0.001 | <0.001 | <0.001 | <0.001 | <0.001 | <0.001 | <0.001 |
| Total terpenes | mg/mL | 0.001 | 0.012 | 0.001 | <0.001 | 0.008 | 0.002 | 0.001 |

This report relates only to the sample(s) and information provided to the laboratory.
Reported results are expressed on an as received basis.
RL = Reporting Limit,
ND = Not Detected

### Trial 10—Collection of Cannabis Vapor at 210° C. Using Glass Fiber Filter Pads

To assess the influence of temperature on analyte recovery as well as the effect of using a filter pad with the vaporizer, a final experiment (Trial 10) was conducted. This set of experiments was performed using broken pellet material loaded in capsules and employing the highest temperature setting of 210° C. on the Mighty vaporizer. Vapor was collected using the filter pad collection setup and extraction procedures described in Trial 7. Experimental parameters are given in Table 34 and results are presented in Tables 35 and 36.

TABLE 34
Vapor Collection Parameters for Trial 10

| | |
|---|---|
| Mass of pellets | 259.1 mg (for cannabinoid trapping) |
| (broken pellets) | 232.3 mg (for terpene trapping) |
| Flow rate through | 0.53 L/min for cannabinoids |
| vaporizer | 0.57 L/min for terpenes |
| Trapping system | 1 filter pad and 4 impingers for cannabinoid trapping |
| | 1 filter pad and 2 impingers for terpene trapping |
| Solvents/volumes | Methanol, 25 mL per impinger, for cannabinoid |
| | trapping Hexane, 10 mL per impinger, for terpene |
| | trapping |
| Vaporizer | 210° C. |
| temperature | |
| Collection time | 6 minutes |

TABLE 35
Cannabinoid Results for Trial 10

| | | Relative |
|---|---|---|
| | Mass recovered from | recovery |
| Analyte | vapor (mg)* | (%)** |
| Total Delta 9-THC (THC + THCA) | 7.75 | 34 |
| Total CBD (CBD + CBDA) | 3.15 | 47 |
| Total CBG (CBG + CBGA) | 0.28 | 29 |
| CBN | 1.43 | (367) |
| CBC | 0.23 | (124) |

*See Certificate of Analysis 293753-OAS (Appendix J).
**Calculated using unburnt pellet extract results as a benchmark.

TABLE 36
Terpene Results for Trial 10

| | Mass recovered from | Relative recovery |
|---|---|---|
| Analyte | vapor (mg)* | (%)** |
| Total Terpenes | 0.16 | 28 |
| Linalool | 0.01 | 14 |
| Terpineol | 0.03 | 65 |
| Caryophyllene | 0.03 | 13 |
| Humulene | 0.01 | 11 |
| Valencene | 0.01 | n/a |
| Trans-nerolidol | 0.02 | n/a |
| Alpha-bisabolol | 0.05 | 108 |

*See Certificate of Analysis 293753-OAS (Appendix J).
**Calculated using unburnt pellet extract results as a benchmark.

Consistent with previous results, it was found that all detectable cannabinoids and terpenes were trapped on the filter pad and not in the impingers. These findings support the use of filter pads for efficient trapping of both cannabis vapor and cannabis smoke. These results also highlight the fact that cannabinoids and terpenes produced from cannabis smoke or vapor are predominantly found in the particulate fraction.

Although the observed color is not as intense for the vapor as that observed for smoke, the filter pad still turned a light yellow after trapping vapor (FIG. 14).

As seen in Table 35, increasing the temperature of the vaporizer from 195° C. to 210° C. resulted in more favorable cannabinoid recoveries than those observed in Trials 8 and 9. These findings are consistent with the previously discussed findings of Pomahacova et al. that demonstrated THC recoveries increased with increasing vaporization temperature [3]. Moreover, the decarboxylation efficiency was comparable to the previous vaporizer trials, with less than 1% of all detected cannabinoids reported in the acid form.

Also, as seen in Table 36, terpene recoveries were improved with higher temperature. Terpenes detected included linalool, terpineol, caryophyllene, humulene, valencene, trans-nerolidol, and alpha-bisabolol. Aside from valencene and trans-nerolidol, all terpenes detected in this round of experiments were also found to be in the dry pellet material.

Cannabinoids in oil by HPLC

| | | | RPC Sample ID: | | | | | |
|---|---|---|---|---|---|---|---|---|
| | | | 293753-04 | 293753-05 | 293753-06 | 293753-07 | 293753-08 | 293753-12 |
| | | | Client Sample ID: | | | | | |
| | | | N11 | N12 | N13 | N14 | N15 | P11 |
| | | | (Cannabis | (Cannabis | (Cannabis | (Cannabis | (Cannabis | (Cannabis |
| | | | Vapor In | Vapor In | Vapor In | Vapor In | Vapor In | Vapor In |
| | | | Methanol) | Methanol) | Methanol) | Methanol) | Methanol) | Methanol) |
| | | | Date Sampled: | | | | | |
| | | | 22 Oct. 2018 | 22 Oct. 2018 | 22 Oct. 2018 | 22 Oct. 2018 | 22 Oct. 2018 | 22 Oct. 2018 |
| | | | Matrix: | | | | | |
| Analytes | Units | RL | other | other | other | other | other | other |
| Delta 9-Tetrahydrocannabinol | mg/ml | 0.001 | 0.310 | ND | ND | ND | <0.001 | 0.347 |
| (Delta 9-THC) | | | | | | | | |
| Delta 9-Tetrahydrocannabinolic | mg/ml | 0.001 | ND | <0.001 | ND | ND | ND | 0.002 |
| Acid (Delta 9-THCA) | | | | | | | | |
| Cannabidiol (CBD) | mg/ml | 0.001 | 0.123 | ND | ND | ND | ND | 0.088 |
| Cannabidiolic Acid (CBDA) | mg/ml | 0.001 | 0.003 | ND | ND | ND | ND | 0.002 |
| Cannabigerol (CBG) | mg/ml | 0.001 | 0.010 | ND | ND | ND | ND | 0.012 |
| Cannabigerolic Acid (CBGA) | mg/ml | 0.001 | 0.001 | <0.001 | ND | ND | ND | <0.001 |
| Cannabinol (CBN) | mg/ml | 0.001 | 0.057 | ND | ND | ND | ND | 0.032 |
| Cannabichromene (CBC) | mg/ml | 0.001 | 0.009 | ND | ND | ND | ND | 0.010 |
| Total Delta 9-Tetrahydrocannabinol | mg/ml | 0.001 | 0.310 | <0.001 | ND | ND | <0.001 | 0.349 |
| (Delta 9-THC) | | | | | | | | |
| Total Cannabidiol (CBD) | mg/ml | 0.001 | 0.126 | ND | ND | ND | ND | 0.090 |
| Total Cannabigerol (CBG) | mg/ml | 0.001 | 0.011 | <0.001 | ND | ND | ND | 0.012 |

This report relates only to the sample(s) and Information provided to the laboratory.
Reported results are expressed on an as received basis.
RL = Reporting Limit,
ND = Not Detected

Cannabinoids in oil by HPLC

| | | | RPC Sample ID: | | | |
|---|---|---|---|---|---|---|
| | | | 293753-13 | 293753-14 | 293753-15 | 293753-16 |
| | | | Client Sample ID: | | | |
| | | | P12 | P13 | P14 | P15 |
| | | | (Cannabis Vapor | (Cannabis Vapor | (Cannabis Vapor | (Cannabis Vapor |
| | | | In Methanol) | In Methanol) | In Methanol) | In Methanol) |
| | | | Date Sampled: | | | |
| | | | 22 Oct. 2018 | 22 Oct. 2018 | 22 Oct. 2018 | 22 Oct. 2018 |
| | | | Matrix: | | | |
| Analytes | Units | RL | other | other | other | other |
| Delta 9-Tetrahydrocannabinol (Delta 9-THC) | mg/ml | 0.001 | ND | ND | ND | ND |
| Delta 9-Tetrahydrocannabinolic Acid (Delta 9-THCA) | mg/ml | 0.001 | ND | ND | ND | ND |
| Cannabidiol (CBD) | mg/ml | 0.001 | ND | ND | ND | ND |
| Cannabidiolic Acid (CBDA) | mg/ml | 0.001 | ND | ND | ND | ND |
| Cannabigerol (CBG) | mg/ml | 0.001 | ND | ND | ND | ND |
| Cannabigerolic Acid (CBGA) | mg/ml | 0.001 | 0.001 | ND | ND | ND |
| Cannabinol (CBN) | mg/ml | 0.001 | ND | ND | ND | ND |
| Cannabichromene (CBC) | mg/ml | 0.001 | ND | ND | ND | ND |
| Total Delta 9-Tetrahydrocannabinol (Delta 9-THC) | mg/ml | 0.001 | ND | ND | ND | ND |
| Total Cannabidiol (CBD) | mg/ml | 0.001 | ND | ND | ND | ND |
| Total Cannabigerol (CBG) | mg/ml | 0.001 | 0.001 | ND | ND | ND |

This report relates only to the sample(s) and information provided to the laboratory.
Reported results are expressed on an as received basis.
RL = Reporting Limit,
ND = Not Detected

Terpenes in cannabis oil

| | | | RPC Sample ID: | | | | | |
|---|---|---|---|---|---|---|---|---|
| | | | 293753-01 | 293753-02 | 293753-03 | 293753-09 | 293753-10 | 293753-11 |
| | | | Client Sample ID: | | | | | |
| | | | N01 | N02 | N03 | P01 | P02 | P03 |
| | | | (Cannabis | (Cannabis | (Cannabis | (Cannabis | (Cannabis | (Cannabis |
| | | | Vapor In | Vapor In | Vapor In | Vapor In | Vapor In | Vapor In |
| | | | Hexane) | Hexane) | Hexane) | Hexane) | Hexane) | Hexane) |
| | | | Date Sampled: | | | | | |
| | | | 22 Oct. 2018 | 22 Oct. 2018 | 22 Oct. 2018 | 22 Oct. 2018 | 22 Oct. 2018 | 22 Oct. 2018 |
| | | | Matrix: | | | | | |
| Analytes | Units | RL | other | other | other | other | other | other |
| Alpha pinene | mg/mL | 0.001 | <0.001 | <0.001 | <0.001 | <0.001 | <0.001 | <0.001 |
| Beta pinene | mg/mL | 0.001 | <0.001 | <0.001 | <0.001 | <0.001 | <0.001 | <0.001 |
| Myrcene | mg/mL | 0.001 | <0.001 | <0.001 | <0.001 | <0.001 | <0.001 | <0.001 |
| Limonene | mg/mL | 0.001 | <0.001 | <0.001 | <0.001 | <0.001 | <0.001 | <0.001 |
| Terpinolene | mg/mL | 0.001 | <0.001 | <0.001 | <0.001 | <0.001 | <0.001 | <0.001 |
| Linalool | mg/mL | 0.001 | 0.001 | <0.001 | <0.001 | <0.001 | <0.001 | <0.001 |
| Terpineol | mg/mL | 0.001 | 0.003 | <0.001 | <0.001 | 0.002 | <0.001 | <0.001 |
| Caryophylene | mg/mL | 0.001 | 0.003 | <0.001 | <0.001 | 0.002 | <0.001 | <0.001 |
| Humulene | mg/mL | 0.001 | 0.001 | <0.001 | <0.001 | <0.001 | <0.001 | <0.001 |
| 3-carene | mg/mL | 0.001 | <0.001 | <0.001 | <0.001 | <0.001 | <0.001 | <0.001 |
| Cis-ocimene | mg/mL | 0.001 | <0.001 | <0.001 | <0.001 | <0.001 | <0.001 | <0.001 |
| Eucalyptol | mg/mL | 0.001 | <0.001 | <0.001 | <0.001 | <0.001 | <0.001 | <0.001 |
| Trans-ocimene | mg/mL | 0.001 | <0.001 | <0.001 | <0.001 | <0.001 | <0.001 | <0.001 |
| Fenchol | mg/mL | 0.001 | <0.001 | <0.001 | <0.001 | <0.001 | <0.001 | <0.001 |
| Borneol | mg/mL | 0.001 | <0.001 | <0.001 | <0.001 | <0.001 | <0.001 | <0.001 |
| Valencene | mg/mL | 0.001 | 0.001 | <0.001 | <0.001 | <0.001 | <0.001 | <0.001 |
| Cis-nerolidol | mg/mL | 0.001 | <0.001 | <0.001 | <0.001 | <0.001 | <0.001 | <0.001 |
| Trans-nerolidol | mg/mL | 0.001 | 0.002 | <0.001 | <0.001 | 0.001 | <0.001 | <0.001 |
| Guaiol | mg/mL | 0.001 | <0.001 | <0.001 | <0.001 | <0.001 | <0.001 | <0.001 |
| Alpha-bisabolol | mg/mL | 0.001 | 0.005 | <0.001 | <0.001 | 0.003 | <0.001 | <0.001 |
| Sabinene | mg/mL | 0.001 | <0.001 | <0.001 | <0.001 | <0.001 | <0.001 | <0.001 |
| Total terpenes | mg/mL | 0.001 | 0.016 | <0.001 | <0.001 | 0.008 | <0.001 | <0.001 |

This report relates only to the sample(s) and information provided to the laboratory.
Reported results are expressed on an as received basis.
RL = Reporting Limit,
ND = Not Detected

### CONCLUSION

The difference between vaporizer and smoking is not what we or anyone would have expected: i.e., we expected that vaporizing results in complete conversion of CBDa to CBD (also applies to other cannabinoids). We did not expect the cannabinoids to be on particulate matter content of the vapor.

One explanation for this is that the pipe design results in a unique environment that aerosolizes the carboxylated cannabinoid (CBDa) and does not completely decarboxylate all of the cannabinoids into their neutral form. The particles in the aerosol (smoke/pipe or vapor/vaporizer) appeared to contain the majority of the cannabinoids and terpenes. We assumed in the case of vaporization, where you're boiling the metabolites, these would leave in their gaseous states and not travel on particles, but this appears to be wrong according to their filter experiments.

Two methods of trapping (impingers and filter pads) were explored in this work and both approaches were found to be effective for recovering cannabinoids and terpenes from cannabis smoke and vapor.

The filter pad approach may offer several advantages over the impinger method. Some advantages may include the fact that the filter pad involves a simpler setup and uses less tubing and solvent. Moreover, the mass of particulate trapped on the filter can be determined quickly by gravimetry. Although recoveries were calculated and reported for all trials, values should be considered rough estimates. Due to limitations of the analytical platform used, flow rates between experiments could not be finely controlled. As a result, one would expect to observe differences in cannabinoid/terpene recoveries between experiments.

### Example 6: Raydiator Pipe and Methods of Use

### Directions for Use

Referring to FIG. 15, twist cover 900 to the left or the right to open the bowl. Add the PPP001 or placebo pellet that was prescribed to you to the bowl. Using a lighter or wooden match, place the flame over the bowl, and then start to lightly inhale from the end of the pipe. Smoke should then begin to go into your mouth and windpipe. Keep smoking and keep cover open. If you are smoking cannabis for the first time, follow the titration method provided by the study nurse the first week, in accordance to your physician's prescription. Then take as many puffs as needed to smoke the content of the bowl. It is recommended to smoke the dose within 15 minutes (dosing period deter-mined with phase 1 clinical trial). If the pipe goes out, you will need to relight, with the same procedure as outlined in step 3.

### Directions for Cleaning

Once you have fully smoked the contents of the pipe, you will need to clean the pipe. Do these cleaning steps once a day. You will see a small amount of resin in the base of the pipe. Just scrape off the bulk of the resin. Drop the pipe into a resealable plastic bag (like a Ziploc®) with 90-100% isopropyl alcohol (available in pharmacy) and a teaspoon of non-iodized salt for a one to two-hour soak. You can also use the isopropyl alcohol without the salt but it will require a good three-hour soak in that case. The non-iodized salt is used as an agitator for a faster cleaning. Let the pipe completely dry prior to re-igniting.

### Example 7: Cannabinoid Recovery from the Vapor of Cannabis Formulations Heated in a Mighty Vaporizer

Intact PPP001 pellets (total of 10) were heated in a Mighty Medic vaporizer at 210° C. for 6 minutes with a pump flow rate 0.75 L/min and vapor was trapped on glass fiber filter cassettes. Both filters and cassettes were extracted with 30 mL of methanol and the extracts analyzed for cannabinoids. Masses and cannabinoid recoveries for pellets are shown in Tables 37 and 38.

TABLE 37
Mass of pellets and mass of particulate
matter trapped on filter cassettes

| | | Particulate mass | Normalized |
|---|---|---|---|
| | | trapped on filter | particulate mass |
| Pellet # | Pellet mass (mg) | cassette (mg) | (mg/g) |
| 1 | 278.0 | 10.8 | 38.8 |
| 2 | 281.6 | 13.5 | 47.9 |
| 3 | 287.4 | 10.7 | 37.2 |
| 4 | 287.4 | 13.5 | 47.0 |
| 5 | 273.2 | 14.2 | 52.0 |
| 6 | 286.8 | 12.9 | 45.0 |
| 7 | 283.2 | 12.1 | 42.7 |
| 8 | 287.0 | 12.0 | 41.8 |
| 9 | 283.8 | 12.1 | 42.6 |
| 10 | 283.2 | 13.5 | 47.7 |

TABLE 38
Masses of cannabinoids recovered from extraction of filters and cassettes

| | Mass recovered (mg) | | | | | | | | | |
|---|---|---|---|---|---|---|---|---|---|---|
| Analyte | 1 | 2 | 3 | 4 | 5 | 6 | 7 | 8 | 9 | 10 |
| Δ-9-THC | 4.45 | 4.66 | 4.58 | 4.53 | 4.75 | 4.73 | 4.72 | 4.51 | 4.74 | 4.43 |
| Δ-9-THCA | ND | ND | ND | ND | ND | ND | ND | ND | ND | ND |
| CBD | 1.62 | 1.73 | 1.71 | 1.66 | 1.76 | 1.75 | 1.66 | 1.65 | 1.67 | 1.59 |
| CBDA | ND | ND | ND | ND | ND | ND | ND | ND | ND | ND |
| CBG | <0.50 | <0.50 | <0.50 | <0.50 | <0.50 | <0.50 | <0.50 | <0.50 | <0.50 | <0.50 |
| CBGA | ND | ND | ND | ND | ND | ND | ND | ND | ND | ND |
| CBN | 0.64 | 0.68 | 0.66 | 0.61 | 0.70 | 0.68 | 0.65 | 0.63 | 0.66 | 0.59 |
| CBC | <0.50 | <0.50 | <0.50 | <0.50 | <0.50 | <0.50 | <0.50 | <0.50 | <0.50 | <0.50 |

ND = Not Detected. See Certificate of Analysis 316735-OAS-MG in Appendix

After heating and collecting vapor for 6 minutes, pellets 1 and 10 were extracted with 30 mL of methanol. The vaporizer mouthpiece and connector tubing were also extracted with 30 mL of methanol for both these pellet runs (see Table 39).

TABLE 39
Masses of cannabinoids recovered from extraction of heated
pellets, vaporizer mouthpieces, and connector tubing

| | Mass recovered (mg) | | | | | |
|---|---|---|---|---|---|---|
| | | Vaporizer | Connector | | Vaporizer | Connector |
| Analyte | Pellet 1 | Mouthpiece | Tubing | Pellet 10 | Mouthpiece | Tubing |
| Δ-9-THC | 7.94 | 1.51 | ND | 8.88 | 2.34 | ND |
| Δ-9-THCA | ND | ND | ND | ND | ND | ND |
| CBD | 3.00 | 0.57 | ND | 3.50 | 0.89 | ND |
| CBDA | ND | ND | ND | ND | ND | ND |
| CBG | 0.68 | <0.50 | ND | 0.74 | <0.50 | ND |
| CBGA | ND | ND | ND | ND | ND | ND |
| CBN | 3.29 | <0.50 | ND | 4.00 | <0.50 | ND |
| CBC | <0.50 | <0.50 | ND | <0.50 | <0.50 | ND |

ND = Not Defected. See Certificate of Analysis 316735-OAS-MG in Appendix

Previous time-course data for PPP001 pellets was collected using broken up pellet material and showed that over 85% of particulate mass collected over 16 minutes of vaporizing was collected within the first 6 minutes (see RPC Reference No. ADM-J1119). It was recognized that the mass transfer dynamics of broken pellets may not be the same as for intact pellets. The mass of particulate matter collected on filter cassettes for two different intact pellets was recorded every two minutes over the course of 30 minutes. The time-course is shown in FIG. 16 and the masses are presented in Table 40.

TABLE 40
Particulate mass collected on filter cassettes at 2-minute intervals

Mass (mg)

| Time (min) | Pellet #1 (0.2744 g) | Pellet #2 (0.2952 g) |
|---|---|---|
| 0 | 0 | 0.0 |
| 2 | 5.6 | 5.9 |
| 4 | 9.1 | 8.3 |
| 6 | 11.6 | 10.6 |
| 8 | 12.9 | 12.1 |
| 10 | 14.3 | 13.2 |
| 12 | 15.1 | 14.1 |
| 14 | 15.6 | 15.0 |
| 16 | 16.3 | 15.8 |
| 18 | 16.9 | 16.5 |
| 20 | 17.3 | 16.9 |
| 22 | 17.7 | 17.4 |
| 24 | 17.9 | 17.6 |
| 26 | 18.1 | 17.9 |
| 28 | 18.1 | 18.4 |
| 30 | 18.2 | 18.2 |

The filters and cassettes for both pellets were extracted with 30 mL methanol and analyzed for cannabinoids to see what the difference in THC/CBD recovery was for a 6-minute vapor collection compared to an exhaustive 30-minute collection. The results are shown in Table 41.

TABLE 41
Masses of cannabinoids recovered from
extraction of filters and cassettes

Mass recovered (mg)

| Analyte | Pellet #1 | Pellet #2 |
|---|---|---|
| Δ-9-THC | 5.68 | 5.28 |
| Δ-9-THCA | ND | ND |
| CBD | 2.79 | 2.72 |
| CBDA | ND | ND |
| CBG | 0.32 | 0.32 |
| CBGA | ND | ND |
| CBN | 2.92 | 2.90 |
| CBC | 0.22 | 0.22 |

ND = Not Detected.
See Certificate of Analysis 317212-OAS-A in Appendix

### Appendix:

Legend for Certificate of Analysis 316735-OAS-MG

| RPC Sample ID | Description |
|---|---|
| 316735-01 | Pellet 1 filter/filter cassette extract |
| 316735-02 | Pellet 2 filter/filter cassette extract |
| 316735-03 | Pellet 3 filter/filter cassette extract |
| 316735-04 | Pellet 4 filter/filter cassette extract |
| 316735-05 | Pellet 5 filter/filter cassette extract |
| 316735-06 | Pellet 6 filter/filter cassette extract |
| 316735-07 | Pellet 7 filter/filter cassette extract |
| 316735-08 | Pellet 8 filter/filter cassette extract |
| 316735-09 | Pellet 9 filter/filter cassette extract |
| 316735-10 | Pellet 10 filter/filter cassette extract |
| 316735-11 | Pellet 1 extract after heating |
| 316735-12 | Vaporizer mouthpiece extract after |
| | heating Pellet 1 |
| 316735-13 | Connector tubing extract after |
| | heating Pellet 1 |
| 316735-14 | Pellet 10 extract after heating |
| 316735-15 | Vaporizer mouthpiece extract after |
| | heating Pellet 10 |
| 316735-16 | Connector tubing extract after |
| | heating Pellet 10 |
| 317212-01 | Pellet 1 filter/filter cassette extract |
| 317212-02 | Pellet 2 filter/filter cassette extract |

Cannabinoids in oil by HPLC

| | | | RPC Sample ID: | | | | |
|---|---|---|---|---|---|---|---|
| | | | 316735-01 | 316735-02 | 316735-03 | 316735-04 | 316735-05 |
| | | | Client Sample ID: | | | | |
| | | | PV-1 | PV-2 | PV-3 | PV-4 | PV-5 |
| | | | Data Sampled: | | | | |
| | | | 5 Jun. 2019 | 5 Jun. 2019 | 5 Jun. 2019 | 5 Jun. 2019 | 5 Jun. 2019 |
| | | | Matrix: | | | | |
| Analytes | Units | RL | other | other | other | other | other |
| Delta 9-Tetrahydrocannabinol (Delta 9-THC) | mg | 0.50 | 4.45 | 4.66 | 4.58 | 4.53 | 4.75 |
| Delta 9-Tetrahydrocannabinolic Acid (Delta 9-THCA) | mg | 0.50 | ND | ND | ND | ND | ND |
| Cannabidiol (CBD) | mg | 0.50 | 1.62 | 1.73 | 1.71 | 1.66 | 1.76 |
| Cannabidiolic Acid (CBDA) | mg | 0.50 | ND | ND | ND | ND | ND |
| Cannabigerol (CBG) | mg | 0.50 | <0.50 | <0.50 | <0.50 | <0.50 | <0.50 |
| Cannabigerolic Acid (CBGA) | mg | 0.50 | ND | ND | ND | ND | ND |
| Cannabinol (CBN) | mg | 0.50 | 0.64 | 0.68 | 0.66 | 0.61 | 0.70 |
| Cannabichromene (CBC) | mg | 0.50 | <0.50 | <0.50 | <0.50 | <0.50 | <0.50 |
| Total Delta 9-Tetrahydrocannabinol (Delta 9-THC) | mg | 0.70 | 4.45 | 4.66 | 4.58 | 4.53 | 4.75 |
| Total Cannabidiol (CBD) | mg | 0.70 | 1.62 | 1.73 | 1.71 | 1.66 | 1.76 |
| Total Cannabigerol (CBG) | mg | 0.70 | <0.70 | <0.70 | <0.70 | <0.70 | <0.70 |

This report relates only to the sample(s) and information provided to the laboratory.
Reported results are expressed on an as received basis.
RL = Reporting Limit,
ND = Not Detected

Cannabinoids in oil by HPLC

| | | | RPC Sample ID: | | | | |
|---|---|---|---|---|---|---|---|
| | | | 316735-06 | 316735-07 | 316735-08 | 316735-09 | 316735-10 |
| | | | Client Sample ID: | | | | |
| | | | PV-6 | PV-7 | PV-8 | PV-9 | PV-10 |
| | | | Date Sampled: | | | | |
| | | | 5 Jun. 2019 | 5 Jun. 2019 | 5 Jun. 2019 | 5 Jun. 2019 | 5 Jun. 2019 |
| | | | Matrix: | | | | |
| Analytes | Units | RL | other | other | other | other | other |
| Delta 9-Tetrahydrocannabinol (Delta 9-THC) | mg | 0.50 | 4.73 | 4.72 | 4.51 | 4.74 | 4.43 |
| Delta 9-Tetrahydrocannabinolic Acid (Delta 9-THCA) | mg | 0.50 | ND | ND | ND | ND | ND |
| Cannabidiol (CBD) | mg | 0.50 | 1.75 | 1.66 | 1.65 | 1.67 | 1.59 |
| Cannabidiolic Acid (CBDA) | mg | 0.50 | ND | ND | ND | ND | ND |
| Cannabigerol (CBG) | mg | 0.50 | <0.50 | <0.50 | <0.50 | <0.50 | <0.50 |
| Cannabigerolic Acid (CBGA) | mg | 0.50 | ND | ND | | ND | ND |
| Cannabinol (CBN) | mg | 0.50 | 0.68 | 0.65 | 0.63 | 0.66 | 0.59 |
| Cannabichromene (CBC) | mg | 0.50 | <0.50 | <0.50 | <0.50 | <0.60 | <0.50 |
| Total Delta 9-Tetrahydrocannabinol (Delta 9-THC) | mg | 0.70 | 4.73 | 4.72 | 4.51 | 4.74 | 4.43 |
| Total Cannabidiol (CBD) | mg | 0.70 | 1.75 | 1.66 | 1.65 | 1.67 | 1.59 |
| Total Cannabigerol (CBG) | mg | 0.70 | <0.70 | <0.70 | <0.70 | <0.70 | <0.70 |

This report relates only to the sample(s) and information provided to the laboratory.
Reported results are expressed on an as received basis.
RL = Reporting Limit,
ND = Not Detected

Cannabinoids in oil by HPLC

| | | | RPC Sample ID: | | | | |
|---|---|---|---|---|---|---|---|
| | | | 316735-11 | 316735-12 | 316735-13 | 316735-14 | 316735-15 |
| | | | Client Sample ID: | | | | |
| | | | EX-1 | EX-2 | EX-3 | EX-4 | EX-5 |
| | | | Date Sampled: | | | | |
| | | | 5 Jun. 2019 | 5 Jun. 2019 | 5 Jun. 2019 | 5 Jun. 2019 | 5 Jun. 2019 |
| | | | Matrix: | | | | |
| Analytes | Units | RL | other | liquid | liquid | other | liquid |
| Dalta 9-Tetrahydrocannabinol (Delta 9-THC) | mg | 0.50 | 7.94 | 1.51 | ND | 8.88 | 2.34 |
| Delta 9-Tetrahydrocannabinolic Acid (Delta 9-THCA) | mg | 0.50 | ND | ND | ND | ND | ND |
| Cannabidiol (CBD) | mg | 0.50 | 3.00 | 0.57 | ND | 3.50 | 0.89 |
| Cannabidiolic Acid (CBDA) | mg | 0.50 | ND | ND | ND | ND | ND |
| Cannabigerol (CBG) | mg | 0.50 | 0.68 | <0.50 | ND | 0.74 | <0.50 |
| Cannabigerolic Acid (CBGA) | mg | 0.50 | ND | ND | ND | ND | ND |
| Cannabinol (CBN) | mg | 0.50 | 3.29 | <0.50 | ND | 4.00 | <0.50 |
| Cannabichromene (CBC) | mg | 0.50 | <0.50 | <0.50 | ND | <0.50 | <0.50 |
| Total Delta 9-Tetrahydrocannabinol (Delta 9-THC) | mg | 0.70 | 7.94 | 1.51 | ND | 8.88 | 2.34 |
| Total Cannabidiol (CBD) | mg | 0.70 | 3.00 | <0.70 | ND | 3.50 | 0.89 |
| Total Cannabigerol (CBG) | mg | 0.70 | <0.70 | <0.70 | ND | 0.74 | <0.70 |

This report relates only to the sample(s) and information provided to the laboratory.
Reported results are expressed on an as received basis.
RL = Reporting Limit,
ND = Not Detected

Cannabinoids in oil by HPLC

| | | | RPC Sample ID: |
|---|---|---|---|
| | | | 316735-16 |
| | | | Client Sample ID: |
| | | | EX-6 |
| | | | Date Sampled: |
| | | | 5 Jun. 2019 |
| | | | Matrix: |
| Analytes | Units | RL | liquid |
| Delta 9-Tetrahydrocannabinol | mg | 0.50 | ND |
| (Delta 9-THC) | | | |
| Delta 9-Tetrahydrocannabinolic | mg | 0.50 | ND |
| Acid (Delta 9-THCA) | | | |
| Cannabidiol (CBD) | mg | 0.50 | ND |
| Cannabidiolic Acid (CBDA) | mg | 0.50 | ND |
| Cannabigerol (CBG) | mg | 0.50 | ND |
| Cannabigerolic Acid (CBGA) | mg | 0.50 | ND |
| Cannabinol (CBN) | mg | 0.50 | ND |
| Cannabichromene (CBC) | mg | 0.50 | ND |
| Total Delta 9-Tetrahydrocannabinol | mg | 0.70 | ND |
| (Delta 9-THC) | | | |
| Total Cannabidiol (CBD) | mg | 0.70 | ND |
| Total Cannabigerol (CBG) | mg | 0.70 | ND |

This report relates only to the sample(s) and information provided to the laboratory.
Reported results are expressed on an as received basis.
RL = Reporting Limit,
ND = Not Detected

Cannabinoids in oil by HPLC

| | | | RPC Sample ID: | | | | | |
|---|---|---|---|---|---|---|---|---|
| | | | 317212-01 | 317212-02 | 317212-03 | 317212-04 | 317212-05 | 317212-06 |
| | | | Client Sample ID: | | | | | |
| | | | EPV 1 | EPV 2 | LDN 1 | LDN 2 | LDN 3 | LDN 4 |
| | | | Date Sampled: | | | | | |
| | | | 10 Jun. | 10 Jun. | 11 Jun. | 11 Jun. | 11 Jun. | 11 Jun. |
| | | | 2019 | 2019 | 2019 | 2019 | 2019 | 2019 |
| | | | Matrix: | | | | | |
| Analytes | Units | RL | other | other | other | other | other | other |
| Delta 9-Tetrahydrocannabinol | mg | 0.05 | 5.68 | 5.28 | 6.32 | ND | 3.28 | ND |
| (Delta 9-THC) | | | | | | | | |
| Delta 9-Tetrahydrocannabinolic | mg | 0.05 | ND | ND | ND | ND | ND | ND |
| Acid (Delta 9-THCA) | | | | | | | | |
| Cannabidiol (CBD) | ma | 0.05 | 2.79 | 2.72 | 2.32 | ND | 1.22 | ND |
| Cannabidiolic Acid (CBDA) | mg | 0.05 | ND | ND | ND | ND | ND | ND |
| Cannabigerol (CBG) | mg | 0.05 | 0.32 | 0.32 | 0.30 | ND | <0.05 | ND |
| Cannabigerolic Acid (CBGA) | mg | 0.05 | ND | ND | ND | ND | ND | ND |
| Cannabinol (CBN) | mg | 0.05 | 2.92 | 2.90 | 0.25 | ND | 0.38 | ND |
| Cannabichromene (CBC) | mg | 0.05 | 0.22 | 0.22 | ND | ND | ND | ND |
| Total Delta 9-Tetrahydrocannabinol | mg | 0.07 | 5.68 | 5.28 | 6.32 | ND | 3.28 | ND |
| (Delta 9-THC) | | | | | | | | |
| Total Cannabidiol (CBD) | mg | 0.07 | 2.79 | 2.72 | 2.32 | ND | 1.22 | ND |
| Total Cannabigerol (CBG) | mg | 0.07 | 0.23 | 0.32 | 0.30 | ND | <0.07 | ND |

This report relates only to the sample(s) and information provided to the laboratory.
Reported results are expressed on an as received basis.
RL = Reporting Limit,
ND = Not Detected

Cannabinoids in oil by HPLC

| | | | RPC Sample ID: | | | |
|---|---|---|---|---|---|---|
| | | | 317212-07 | 317212-08 | 317212-09 | 317212-10 |
| | | | Client Sample ID: | | | |
| | | | LDN 5 | LDN 6 | LDN 7 | LDN 8 |
| | | | Date Sampled: | | | |
| | | | 11 Jun. 2019 | 11 Jun. 2019 | 11 Jun. 2019 | 11 Jun. 2019 |
| | | | Matrix: | | | |
| Analytes | Units | RL | other | other | other | other |
| Delta 9-Tetrahydrocannabinol (Delta 8-THC) | mg | 0.05 | 6.24 | ND | 3.31 | ND |
| Delta 9-Tetrahydrocannabinolic Acid (Delta 9-THCA) | mg | 0.05 | ND | ND | ND | ND |
| Cannabidiol (CBD) | mg | 0.05 | 2.31 | ND | 1.11 | ND |
| Cannabidiolic Acid (CBDA) | mg | 0.05 | ND | ND | ND | ND |
| Cannabigerol (CBG) | mg | 0.05 | 0.30 | ND | ND | ND |
| Cannabigerolic Acid (CBGA) | mg | 0.05 | <0.05 | ND | ND | ND |
| Cannabinol (CBN) | mg | 0.05 | 0.22 | ND | 0.26 | ND |
| Cannabichromene (CBC) | mg | 0.05 | ND | ND | ND | ND |
| Total Delta 9-Tetrahydrocannabinol (Delta 9-THC) | mg | 0.07 | 6.24 | ND | 3.31 | ND |
| Total Cannabidiol (CBD) | mg | 0.07 | 2.31 | ND | 1.11 | ND |
| Total Cannabigerol (CBG) | mg | 0.07 | 0.30 | ND | ND | ND |

This report relates only to the sample(s) and information provided to the laboratory.
Reported results are expressed on an as received basis.
RL = Reporting Limit,
ND = Not Detected

### Example 8: PPP-011

Compatibility of the Drug Substance with the Excipients

There may be no excipients in the finished drug product. Exemplary embodiments comprise of pure synthetic THC and synthetic CBD contained within a stainless steel and aluminum drip dosing capsule. Ethanol may be used to prepare the intermediate liquid formulation and, in some cases, be considered an excipient.

To deposit the synthetic THC and CBD onto the drip pad, they may be dissolved in a solution. In their pure forms, CBD is a white to yellowish powder and THC is an oily, sticky resin. Both cannabinoids are not soluble in water but are soluble in ethanol. Solvents such as ethanol may offer protection against degradation, especially for THC, which is known to be particularly labile. THC is more stable in ethanol than it is in other solvents such as carbon tetrachloride or hexane (NTP, 1996). Despite the added stability provided by ethanol, the THC in ethanol (raw material) and the PPP011 intermediate liquid formulation should not be handled in the open air during the manufacturing procedure. All manufacturing activities involving these solutions may occur under nitrogen.

### Physicochemical Characteristics of the Drug Substance

The low aqueous solubility of THC and CBD and the sensitivity of THC to oxygen are two characteristics that may affect the performance and manufacturability of PPP011.

### Solubility

THC and CBD are highly lipophilic molecules with very low aqueous solubility (Grotenhermen, 2003). The log P values (where P is the octanol/water partition coefficient) for THC and CBD are 6.99 and 5.79, respectively (United Nations Office on Drugs and Crime, 2009). They are also small molecules with identical molecular weights of 314.36 g/mol. These two properties (high lipid solubility and molecular weights less than 400 g/mol) may allow them to cross the blood brain barrier via lipid-mediated free diffusion (Pardridge, 2012). Ability to access the central nervous system contributes to the analgesic and euphoric effects of cannabinoids.

PPP011 may be manufactured by loading a liquid formulation of THC, CBD, and ethanol into a drip dosing capsule and subsequently evaporating the ethanol. The low aqueous solubility of THC and CBD was a factor in the choice of carrier liquid for the intermediate formulation—it was restricted to either an oil or a solvent. Ethanol was chosen for these embodiments because it may be removed from the finished drug product.

### Sensitivity to Heat and Oxygen

Heat, light, humidity, acidity, and oxidation may affect the stability of cannabis and cannabinoids (Garrett and Hunt, 1974; Fairbairn et al., 1976). Use of heat and exposure to oxygen are avoided during the PPP011 manufacturing procedure. Manufacturability of PPP011 may be affected by the need to avoid oxygen exposure throughout the manufacturing process. This is achieved by working under nitrogen for at least part of the procedure, including the packaging of the drug product. In some embodiments, all of the manufacturing process is conducted under nitrogen or vacuum.

### Excipients

The drug product may be free of excipients. It may comprise pure synthetic THC and synthetic CBD contained within a stainless steel and aluminum drip dosing capsule. Ethanol may be used to prepare the intermediate liquid formulation and can therefore be considered an excipient. However, because it is present at a very low level in the finished drug product (0.2 mg per dose), it does not affect the performance of the drug. Ethanol is the only excipient used in the PPP011 manufacturing process, so may only interact with the drug substances and not other excipients.

### Formulation Development

PPP011 is a vaporized drug that delivers the same amount of THC and CBD as vaporized PPP001, a plant-based investigational drug developed by Tetra Bio-Pharma. The initial concept for PPP011 was to create a drug that was similar to PPP001 in terms of intended usage, administration route, and cannabinoid dose, but with a cleaner formulation. The final PPP011 formulation is excipient-free, composed only of synthetic THC and CBD, which may offer several advantages over PPP001 including reduced potential for the formation of by-products generated by burning or heating cannabis plant material; reduced potential for microbial contamination, which may be present in plant-based drugs; and more flexibility in controlling absolute amounts and ratios of THC and CBD in the drug. Other advantages may include single use formulations, as the synthetic THC and CBD may be consumed in a single use. This may also aid in decreasing the potential for abuse.

### Comparison of PPP011 and PPP001

Table 42 compares the formulation, dose, and administration of PPP011 and PPP001.

TABLE 42
Comparison of PPP011 and PPP001

| Attribute | PPP011 (synthetic drug formulation) | PPP001 (cannabis plant pellet) |
|---|---|---|
| Source of drug | Synthetic cannabinoids | Cannabis plant |
| substance | | |
| Drug substance | Synthetic THC (in ethanol) + synthetic | 3 strains of cannabis dried and milled |
| | CBD (crystalline) | individually, then blended together |
| Dosage form | Stainless steel/aluminum drip dosing | 280 mg pellet of compressed cannabis |
| | capsule containing pure THC and CBD | blend |
| | (no excipients) | |
| Dose | 17 mg THC + 5 mg CBD | 26.6 mg THC (9.5%) + 7 mg CBD |
| | | (2.5%) (+other cannabinoids and |
| | | terpenes in plant) |
| Administration | Inhalation (vaping only) | Inhalation (vaping or smoking) |
| route | | |
| Device for | Mighty Medic ® vaporizer | Mighty Medic ® vaporizer for vaping or |
| administration | | titanium pipe for smoking |

As Table 42 indicates, the doses of PPP011 and PPP001 may not be equivalent. However, when cannabis plant material is smoked or vaporized, only a percentage of the cannabinoids are recovered in the smoke or vapor (Gieringer et al., 2004; Hazekamp et al., 2006; Pomahacova et al., 2009); this is also the case when synthetic THC and/or CBD is vaporized (Hazekamp et al., 2006; Solowij et al., 2014). PPP011 may be formulated to deliver the same amount of THC and CBD as PPP001 to the subject via the smoke/vapor. To this end, experiments were carried out to determine the percentage of THC and CBD that entered the vapor when each drug product was vaporized using the Mighty Medic®. Subsequently, calculations were performed to determine the amount of THC and CBD needed in PPP011 such that the vapor produced by that formulation contained the same amount of THC and CBD as the vapor of PPP001.

FIG. 17 shows a visual comparison of PPP011 (synthetic cannabinoids) and PPP001 (cannabis plant pellet), as well as photographs of the Mighty Medic® vaporizer. Panel A shows the top and side view of a drip dosing capsule, which is composed of a stainless steel drip pad placed inside an aluminum dosing capsule. The drip dosing capsule is loaded into the filling chamber of the Mighty Medic® vaporizer. PPP011 may be manufactured by loading a liquid formulation of THC, CBD, and ethanol into the drip dosing capsule, followed by evaporation of the ethanol. Panel B shows the PPP001 cannabis plant pellet packaged inside a blister pack. As pictured in Panels C and D, both the drip dosing capsule and the plant pellet fit into the filling chamber of the Mighty Medic®. To access the filling chamber, the cooling unit of the Mighty Medic® is removed (Panel E). To inhale the drug, the cooling unit is re-mounted and the mouthpiece is flipped out (compare Panels F and G).

### Justification of PPP011 Delivery Method, Delivery Device, Packaging, and Formulation

The clinical development program for improving quality of life and managing pain in patients with advanced cancer was initiated with PPP001, which may comprise three cannabis strains that are dried and milled, then blended together in a fixed ratio and compressed into a pellet suitable for smoking or vaping (see Table 42 and FIG. 17). PPP011 represents an improvement upon the original plant-based formulation.

### Vaporization (vs Smoking) as Delivery Method

The decision to pursue a vaporized drug product was based on safety. Vaping has been suggested as a safer intrapulmonary delivery system than smoking since it involves heating (as opposed to combusting) cannabis, thereby avoiding the formation of pyrolytic toxic compounds such as carbon monoxide and carcinogens (Solowij, 2018). Indeed, studies comparing the content of cannabis smoke and cannabis vapor have shown that the number of harmful, non-cannabinoid compounds is drastically reduced in vapor vs smoke (Gieringer, 2001; Gieringer et al., 2004). Similarly, when the smoke and vapor of PPP001 were analyzed, numerous toxic compounds present in the smoke (including formaldehyde, carbon monoxide, ozone, and benzene) were not found in the vapor (see Table 44).

### Drip Dosing Capsules and Mighty Medic® for Drug Packaging and Delivery

The Mighty Medic® is used herein for clinical trials because it is currently the only battery-powered medical vaporizer manufactured in compliance with medical device quality requirements. In Canada, it is certified as a Class 2 medical device (licence number 96431). Storz & Bickel is the only company worldwide to produce medical herbal vaporizers and it currently has three models: the Volcano Medic®, the Volcano Medic 2@, and the Mighty Medic® (Storz & Bickel, 2019a). The Volcano vaporizers are not considered portable—they require a power cord and weigh approximately four pounds. Thus, the Mighty Medic® may represent a more convenient option for patients.

The drip dosing capsules are a commercial product available from Storz & Bickel and are designed to function with the Mighty Medic®. When the Mighty Medic® was approved as a medical device, the drip dosing capsules were included in this approval. The drip pad and dosing capsule are made of stainless steel and aluminum, respectively, both of which are ideal because they are low-VOC materials that may tolerate a temperature of 210° C. without the risk of ignition.

### Mylar Foil Bags for Drug Packaging

Pure THC is an oily, sticky resin that is known to be labile. It is unstable in air, light, and at high temperatures, and it may be less stable as a thin film than it is in solution (Mechoulam, 1970; National Institutes of Health, 1996). Because of these properties of THC, PPP011 may be manufactured under nitrogen. Furthermore, each drip dosing capsule may be individually packaged under nitrogen so that it is not exposed to oxygen until the patient is ready to use it. One method is to package each drip dosing capsule in its own small mylar foil bag and seal under nitrogen. As an added layer of protection, groups of 24 small mylar foil bags (a one-week supply of the drug) are then placed inside a large mylar foil bag, which is also sealed under nitrogen. This double layer of protection may remain intact during storage and shipping. Both the large and small mylar foil bags are 4.3 mils thick and have an extremely low oxygen transmission rate of 0.001/cc/m2/24 hours. Other primary packaging, secondary packaging, scavengers or sensors as described herein may be used.

### Synthetic (vs Plant-Based) Cannabinoids as Raw Materials

The development of PPP011 involved switching from a plant-based vaporized drug to a synthetic one. One advantage of this switch is reduced potential for microbial contamination. Cannabis plants cannot be grown under conditions that are sterile enough to keep microbe levels below the required safety limits. Even if they could be grown this way, maintaining sterility would be difficult throughout the other steps in the production process (harvesting, drying, processing). For patient safety, Canadian regulations require that medical cannabis is treated with gamma irradiation before it is dispensed (Hazekamp, 2016). Irradiation eliminates bacteria and fungi from the final product. This is an important practice, since inhaled microbes, particularly fungi, may be fatal for immunocompromised individuals who may be using cannabis to achieve relief from their condition (e.g., cancer patients). In these patients, pulmonary fungal infections may become invasive and result in death (Hazekamp, 2016; Stone et al., 2019). In contrast to relying on an isolated step in the manufacturing process to produce raw materials that are free of microbes (i.e., irradiation), the quality and purity of the raw materials for PPP011 can be controlled throughout the manufacturing process since they are chemically synthesized.

In addition to causing infections, fungi produce a variety of mycotoxins, some of which are classified as Group 1 human carcinogens (Di Stefano et al., 2014). In products meant for animal or human consumption (e.g., foods, medicinal herbs), mycotoxins are notoriously difficult to degrade completely without also destroying the nutritional and functional qualities of the product. There are conflicting results on the ability of gamma irradiation to degrade mycotoxins in food, which may be due to the moisture content of the food in question and the particular toxin being tested (Doyle et al., 1982; Lucas, 2008; Aquino, 2011; Di Stefano et al., 2014). Thus, as demonstrated in, for example, Tetra Bio-Pharma, 2019, even irradiated cannabis products may not be mycotoxin-free. Because PPP011 is made with synthetic THC and CBD rather than cannabis plant material, the risk of contamination from microbes or mycotoxins is extremely low. As shown below, of the bacteria measured in the PPP011 drug product, all were absent, and yeasts/molds were below the specified limit.

### Ethanol (vs Oil) as Carrier Liquid

An important decision for PPP011 was the choice of carrier liquid for the intermediate formulation. To deposit the synthetic THC and CBD onto the drip pad, the cannabinoids may be dissolved in a solution; in their pure forms, CBD is a white to yellowish powder and THC is an oily, sticky resin. THC and CBD are not soluble in water (with log P values of 6.99 and 5.79, respectively), so initially, an oil was considered. In this scenario, patients would have dispensed the drug into the dosing capsules themselves. Control of dosing would have been more difficult to achieve using this approach rather than the chosen approach, which involves pre-loading the drip dosing capsules with the drug.

An additional reason why oil was not used as the carrier liquid was lack of information on the safety of vaping various oils. Most of the studies on the safety of liquid formulations for vaping have been performed with solutions known as “e-liquids,” which are used in electronic cigarettes (e-cigarettes). These e-liquids typically contain compounds such as propylene glycol and vegetable glycerine, which, when heated, produce vapor that contains carcinogens and respiratory irritants (e.g., formaldehyde, acetaldehyde, acrolein) (Kosmider et al., 2014; Sleiman et al., 2016; Ogunwale, Li, et al., 2017). While some commercially available cannabis oils designed for vaping also contain propylene glycol and/or glycerine as thinning agents, others use MCT oil, which is made by extracting medium chain triglycerides from coconut or palm oil. MCT oil is often ingested as food or as a nutritional supplement, and is sometimes touted as a safe, healthy alternative to other thinning agents in cannabis oil; however, the potential health effects of vaporizing MCT are largely unstudied (Troutt and DiDonato, 2017).

In addition to the general lack of information on vaporized MCT, another safety concern was the possibility of exogenous lipoid pneumonia (ELP). This is an extremely rare condition that is caused by aspiration or inhalation of lipid particles into the lung and has most often been associated with the use of mineral oil as a laxative. It can also be caused by nasal administration of oily substances and by inhalation of vaporized oils. Most cases of ELP have been caused by mineral or animal oils, but vegetable oils have been involved in a small number of cases (e.g., olive oil, peanut oil, sesame oil) (Gondouin et al., 1996; Hadda and Khilnani, 2010).

There is one documented case of ELP in a patient who inhaled vaporized cannabis oil 2-10 times per day for 10 years. The oil was prepared by mixing a cannabis extract with petroleum jelly, vitamin E, or another oil-based substance (Vethanayagam et al., 2000). It has been suggested that inhalation of MCT oil vapor is less likely to cause ELP than other oils. Although possibly a safer oil for vaporization, MCT oil was deemed an inappropriate carrier liquid for PPP011 and ethanol was instead chosen.

Ethanol is an ideal carrier liquid for PPP011 because it can be evaporated, resulting in a drug product with no excipients, except for a very small amount of residual ethanol. Thus, any concerns related to the safety of a given carrier liquid are reduced.

### Evidence of a Cleaner Vapor for PPP011: VOCs in the Vapor of PPP011 vs PPP001

The minimal, excipient-free formulation of PPP011 (current investigational drug) results in a cleaner vapor with fewer VOCs than the vapor of PPP001 (plant pellet). This was demonstrated by heating PPP011 drip dosing capsules and PPP001 plant pellets in a Mighty Medic® and analyzing the vapor using non-targeted VOC screening via gas chromatography mass spectrometry (GC-MS). These results are summarized in Tables 44 and 45. Analytical Platform and Screening Method

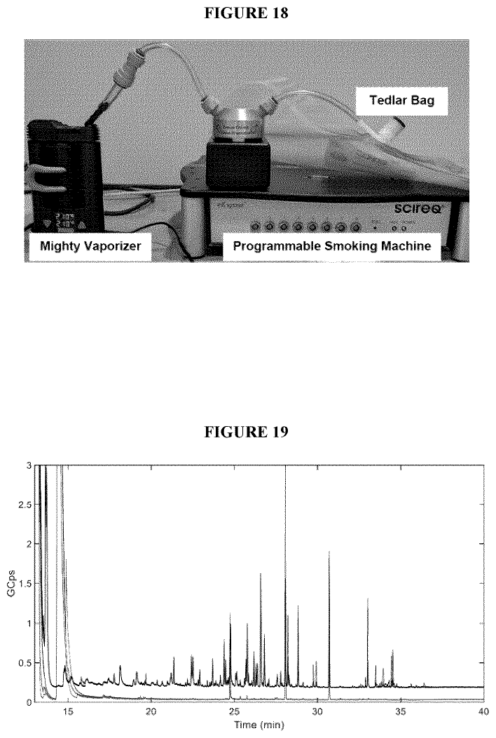

A method was developed by RPC to collect the PPP011 and PPP001 vapor in a collection vessel suitable for sampling via GC-MS. The GC-MS instrument was equipped with a sample pre-concentration trap (SPT) and capillary column (analytical column). This allows for a large sample volume to be injected and then concentrated on the SPT prior to injection onto the analytical column. Thus, molecules in the vapor are concentrated, enabling increased limits of detection. A sample volume of 60 mL loaded onto the SPT was found suitable for VOC profiling of the vapor. The vapor collection system consisted of a Mighty Medic®, a programmable pump, Tygon tubing, and a Tedlar bag (FIG. 18). The vaporizer was set to 210° C. and vapor from PPP011 or PPP001 was collected into the Tedlar bag for 6 minutes at a flow rate of 0.75 L/min. Three samples were run for each drug product.

The experiments conducted in this study involved a non-targeted screening approach. Non-targeted screening is employed when it is not known what VOCs are present in the sample matrix. For this reason, there are no standards to compare against the unknown spectra as one cannot predict what standards to purchase prior to conducting the screening experiments. Thus, non-targeted screening relies heavily on library (database) searching. For this study, VOCs in the samples were tentatively identified by searching a National Institute of Standards and Technology (NIST) database of compounds. Where possible, database matches were confirmed with the retention time from a reference standard.

The GC-MS instrument can detect very low amounts of analyte; therefore, if a harmful (or potentially harmful) compound is detected, it does not necessarily mean it is present in an amount that could cause harm. For example, normal metabolic processes in humans produce chemicals that are detectable as VOCs in exhaled breath. Some VOCs present in the breath of healthy individuals are isoprene, acetone, ethanol, methanol, and acetaldehyde (Fenske and Paulson, 1999).

### Results: VOCs Detected in PPP011 vs PPP001

Table 44 and Table 45 list the VOCs identified in the vapor of PPP001 and PPP011. In some cases, compound identifications could be confirmed by cross referencing the tentative identification with a standard. In these cases, the compound name is highlighted in green. Using 60 mL samples loads for both drug products, 100 VOCs were identified in the vapor of PPP001 whereas only 8 VOCs were identified in the vapor of PPP011. FIG. 19, which shows an overlay of the PPP001 (black) and PPP011 (red) vapor chromatograms, provides a visual representation of the cleaner PPP011 vapor.

TABLE 44
VOCs Identified in Vapor of PPP001
Plant Pellets (60 mL Sample Loads)

| Name | CAS # |
|---|---|
| Acetaldehyde | 75-07-0 |
| Acetone | 67-64-1 |
| Acetic acid, methyl ester | 79-20-9 |
| Methyl nitrate | 598-58-3 |
| Propanal, 2-methyl- | 78-84-2 |
| Glutaraldehyde | 111-30-8 |
| 1,3-Pentadiene, 2-methyl-, (E)- | 926-54-5 |
| 3-Buten-2-ol, 2-methyl | 115-18-4 |
| Butanal, 3-methyl- | 590-86-3 |
| Butanal, 2-methyl- | 96-17-3 |
| 2,4-Hexadienal, (E,E)- | 142-83-6 |
| 2-Penten-1-ol, (E)- | 1576-96-1 |
| 1-Ethylcyclopropanol | 57872-31-8 |
| 1,2-CyclopentanedioI, 3-methyl- | 27583-37-5 |
| 2-Vinylfuran | 1487-18-9 |
| 2-Hexenal, (E)- | 6728-26-3 |
| Bicyclo[2.2.1]hept-2-en-7-ol | 53783-87-2 |
| Disulfide, dimethyl | 624-92-0 |
| Pyridine | 110-86-1 |
| 3-Hexanone | 589-38-8 |
| Prenol | 556-82-1 |
| l,5-Dimethyl-6-oxa- | 82461-31-2 |
| bicyclo[3.l.0]hexane | |
| Pyrrole | 109-97-7 |
| Hexanal | 66-25-1 |
| Pyridine, 3-methyl- | 108-99-6 |
| Bicyclo[3.l.l]hept-2-en-6-one, 2,7,7- | 473-06-3 |
| trimethyl- | |
| Pyrazine, methyl- | 109-08-0 |
| 2,3-Butanediol | 513-85-9 |
| DL-2,3-Butanediol | 6982-25-8 |
| 3-Decyn-2-ol | 69668-93-5 |
| 2-Nonen-1-ol, (E)- | 31502-14-4 |
| Ethanone, 1-(3-methylenecyclopentyl)- | 54829-98-0 |
| 1-Undecyne | 2243-98-3 |
| 1 H-Pyrrole, 2,3-dimethyl- | 600-28-2 |
| Bicyclo[2.2.l]hept-2-ene, 1,7,7- | 464-17-5 |
| trimethyl- | |
| 1 H-Pyrrole, 2-methyl- | 636-41-9 |
| 2-Furanmethanol | 98-00-0 |
| 3-Methylcyclopentyl acetate | 24070-70-0 |
| 2-Heptanone | 110-43-0 |
| Bicylco[3.1.0]hex-2-ene, 2-methyl-5- | 2867-05-02 |
| (1-methylethyl)- | |
| Heptanal | 111-71-7 |
| Pyrazine, 2,6-dimethyl- | 108-50-9 |
| Bicyclo[2.2.l]heptane, 2,2-dimethyl-3- | 5794-03-06 |
| methylene, (lR)- | |
| Camphene | 79-92-5 |
| Bicyclo[3.1.0]hex-2-ene, 4-methylene- | 36262-09-6 |
| 1-(1-methylethyl)- | |
| cis-2,6-Dimethyl-2,6-octadiene | 2492-22-0 |
| Furan, 2-pentyl- | 3777-69-3 |
| Dimethyl trisulfide | 3658-80-8 |
| 2,3-Dehydro-1,8-cineole | 92760-25-3 |
| (2R,5S)-2-Methyl-5-(prop-1-en-2-yl)- | 54750-69-5 |
| 2-vinyltetrahydrofuran | |
| 2,6-Dimethyl-1,3,5,7-octatetraene, E,E- | 460-01-5 |
| 5-Hepten-2-one, 6-methyl- | 110-93-0 |
| 2-Carene | 554-61-0 |
| 1,3,8-p-Menthatriene | 18368-95-1 |
| m-Cymene | 535-77-3 |
| beta-Phellandrene | 555-10-2 |
| gamma-Terpinene | 99-85-4 |
| Linalool oxide | 5989-33-3 |
| cis-Sabinene hydrate | 15537-55-0 |
| Fenchone | 1195-79-5 |
| Cyclopentane, nitro- | 2562-38-1 |
| Ylangene | 14912-44-8 |
| Aromandendrene | 489-39-4 |
| alpha-Santalene | 512-61-8 |
| trans-alpha-Bergamotene | 13474-59-4 |
| Cedren-13-ol, 8- | 18319-35-2 |
| beta-Curcumene | 28976-67-2 |
| alpha-Gurjunene | 489-40-7 |
| beta-Bisabolene | 495-61-4 |
| alpha-Guaiene | 3691-12-01 |
| Sesquicineole | 90131-02-5 |
| beta-Longipinene | 41432-70-6 |
| cis-alpha-Bisabolene | 29837-07-8 |
| gamma-Gurjunene | 22567-17-5 |
| beta-Guaiene | 88-84-6 |
| (4aR,8aS)-4a-Methyl-1-methylene-7- | 58893-88-2 |
| (propan-2-ylidene)decahydronapthalene | |
| Selina-3,7(11)-diene | 6813-21-4 |
| Nerolidol | 142-50-7 |
| Caryophyllene oxide | 1139-30-6 |
| gamma-Selinene | 515-17-3 |
| alpha-Acorenol | 28296-85-7 |

TABLE 45
VOCs Identified in Vapor of PPP011 Drip
Dosing Capsules (60 mL Sample Loads)

| Name | CAS # |
|---|---|
| Acetaldehyde | 75-07-0 |
| Butanal | 123-72-8 |
| 1-Butanol | 71-36-3 |
| 2-Heptanone | 110-43-0 |
| 2,6-Dimethyl-1,3,5,7-octatetraene, | 460-01-5 |
| E,E- | |
| 1,3,8-p-Menthatriene | 18368-95-1 |
| 2-Cyclohexen-1-one, 3-methyl-6-(1- | 491-09-8 |
| methylethylidene)- | |

Of the 8 VOCs listed in Table 45 for the PPP011 vapor, 4 were also found in the vapor of PPP001 plant pellets (acetaldehyde, 2-heptanone, 2,6-dimethyl-1,3,5,7-octatetraene, E,E-, and 1,3,8-p-menthatriene). Of all the peaks in the PPP011 chromatograms, the ethanol peaks eluting at 14.4 minutes were by far the largest, with the largest peak exceeding 25 GCps. This is a reflection of the method used to evaporate the ethanol from the drip dosing capsules after loading them with the intermediate formulation. Embodiments of the manufacturing process for clinical batches involve drying the drip dosing capsules in a vacuum oven; however, when the VOC study was conducted, the drip dosing capsules were left in a glove bag (purged and filled with nitrogen) under a gentle stream of nitrogen for at least 90 minutes to remove ethanol. This latter process is less efficient than the vacuum oven, which explains the large ethanol peak in the PPP011 samples.

When the drip dosing capsules are dried in the vacuum oven, there is approximately 0.2 mg of residual ethanol per capsule. Studies have not yet been performed to quantify the level of ethanol in the vapor of PPP011, but based on the amount remaining in each capsule, the level would be very low. To put this value of 0.2 mg into perspective, consider the following: a study with ethanol-based hand sanitizer measured ethanol in the vapor released during hand disinfection. The authors then calculated exposure to inhaled ethanol by combining the measured ethanol concentration in the vapor, average breathing frequency, and average tidal volume. During a 30-second hand disinfection, the inhaled dose of ethanol was approximately 75 mg (Bessonneau and Thomas, 2012). Healthcare workers sanitize their hands as many as 30 times per shift (Boyce and Pittet, 2002), which would result in a daily inhaled dose of 2,250 mg of ethanol. If all the residual ethanol on the PPP011 drip pad was inhaled by the patient, the daily inhaled dose of ethanol from the drug product would be 0.6 mg (0.2 mg per dose×3 doses), which is 3,750 times less than the daily dose of inhaled ethanol for a healthcare worker who sanitizes their hands 30 times.

To see if less concentrated VOC components could be identified in the vapor of PPP011, samples were re-run with the same GC-MS method used for 60 mL sample loads, but with a larger loading volume of 720 mL. Increasing the sample volume allowed for the tentative identification of 15 additional VOCs (total of 23 identified VOCs; see Table 45-2, below). Thus, even with a PPP011 vapor sample that was 12 times larger than the PPP001 sample, substantially fewer VOCs were detected for PPP011. These data indicate that PPP011 produces a significantly cleaner vapor than PPP001.

TABLE 45-2
VOCs identified in vapor of PPP011 capsules, 720 ml sample loads

| Name | CAS # |
|---|---|
| Propane, 1-chloro-2-methyl- | 513-36-0 |
| Acetaldehyde | 75-07-0 |
| Butanal | 123-72-8 |
| 1,2-Propanediol, 3-methoxy- | 623-39-2 |
| 1-Butanol | 71-36-3 |
| Pentanal, 3-methyl- | 15877-57-3 |
| 2-Octanone, 1-nitro | 16067-01-9 |
| 3-Heptanone | 106-35-4 |
| 2-Heptanone | 110-43-0 |
| 3-Hexanol, 5-methyl- | 623-55-2 |
| Tetraethyl silicate | 78-10-4 |
| 2,6-Dimethyl-1,3,5,7-octatetraene, E,E- | 460-01-5 |
| 5-Hepten-2-one, 6-methyl- | 110-93-0 |
| 1,3,8-p-Menthatriene | 18368-95-1 |
| Isobutyl ether | 628-55-7 |
| Benzene, 1-methyl-4-(1-methylethenyl)- | 1195-32-0 |
| Benzene, 1-methyl-3-(1-methylethenyl)- | 1124-20-5 |
| 1,3,8-p-Menthatriene | 18368-95-1 |
| p-Mentha-1,5,8-triene | 21195-59-5 |
| 2-Cyclohexen-1-one, 3-methyl-6-(1-methylethylidene)- | 491-09-8 |
| Butylated Hydroxytoluene | 128-37-0 |
| Dodecanoic acid, 1-methylethyl ester | 10233-13-3 |

### Evidence of Equivalent Dosing: Cannabinoid Levels in the Vapor of PPP011 vs PPP001

PPP011 (current investigational drug) was designed to deliver the same amount of THC and CBD as vaporized PPP001 (cannabis plant pellet), but with a cleaner formulation. As shown in Table 43, the doses of cannabinoids in PPP011 and PPP001 are not equivalent. PPP011 contains 17 mg synthetic THC and 5 mg synthetic CBD, whereas PPP001 contains 26.6 mg THC and 7 mg CBD. However, the level of THC and CBD in the vapor of these two drug products is equivalent for the following reason: when cannabis plant material or synthetic cannabinoids are vaporized, only a percentage of the cannabinoids are recovered in the vapor (Gieringer et al., 2004; Hazekamp et al., 2006; Pomahacova et al., 2009; Solowij et al., 2014; Osborne et al., 2017). As discussed below, the relative recoveries of THC and CBD in the vapor of PPP011 and PPP001 are different—they are higher for PPP011. Thus, in order for the drugs to produce vapor with equivalent amounts of THC and CBD, a lower loading dose of cannabinoids is required for PPP011.

To choose the dose of PPP011, experiments were carried out to determine the percentage of THC and CBD that entered the vapor when PPP001 and PPP011 were vaporized using the Mighty Medic®. Subsequently, calculations were performed to determine the amount of THC and CBD needed in PPP011 such that the vapor produced by that formulation contained the same amount of THC and CBD as the vapor of PPP001. This was an iterative process, involving several rounds of experiments, each with PPP011 formulations of different concentrations. Results from all the experiments were used to construct a curve of “recovery in vapor” vs “loading dose” to determine the required dose for PPP011.

### Experimental Set-up for Vapor Analysis

The system shown in FIG. 4 was used to capture the cannabinoids in the vapor of PPP001 and PPP011. A smoking machine, consisting of a software-programmable air pump and pump controller unit, draws vapor produced by the Mighty Medic® through a filter cassette, which traps the cannabinoids. The cannabinoids are extracted from the filter cassette in methanol and the extract is then analyzed by high performance liquid chromatography (HPLC) to quantify the cannabinoids.

Examples 1-5 detail the development of the vapor collection system. Solvent-filled impingers and glass fiber filter pads were explored as methods for trapping cannabinoids in the vapor. Both methods were effective, but the filter pad approach offered several advantages over the impinger method, including the ability to easily track the mass of particulate on the filter by gravimetry. Pump flow rates were very repeatable, with a relative standard deviation of less than 3%. Pump accuracy was maintained, with a percent difference between set and measured flow rates of less than 5%. These results demonstrated that the smoking machine was ideal for collection of samples for quantitative analysis.

To accurately compare cannabinoid levels in the vapor of PPP001 and PPP011, it was first necessary to determine how long it took to completely vaporize each drug product. With the vaporizer set to 210° C. and the pump flow rate at 0.75 L/min (the conditions used for all vapor collection studies), the mass of particulate matter collected on the filter cassettes was measured at 2-minute intervals. Earlier preliminary testing had indicated that PPP001 (cannabis plant pellet) took longer to vaporize than PPP011 (drip dosing capsules); therefore, PPP001 was vaporized for a total of 30 minutes and PPP011 for 14 minutes. For PPP001, approximately 95% of particulate mass collected over 30 minutes was collected within the first 20 minutes. For PPP011, approximately 95% of particulate mass collected over 14 minutes was collected within the first 6 minutes. Thus, for analyses of cannabinoid levels in the vapor of each drug product, PPP001 was vaporized for 20 minutes and PPP011 for 6 minutes.

Clinical batches of PPP011 are produced by drying the drip dosing capsules in a vacuum oven after they are loaded with the liquid formulation. This results in a very low level of residual ethanol (0.2 mg per dose). For the vapor collection studies at RPC, the drip dosing capsules were prepared in the following way:
Liquid formulations were opened inside a glove bag purged and filled with nitrogen.
The liquid formulation (100 μl) was pipetted into each drip dosing capsule while working inside the glove bag.
The drip dosing capsules were left in the glove bag under a gentle stream of nitrogen for at least 90 minutes to remove ethanol.
The drip dosing capsules were placed in the Mighty Medic® and vaporized for 6 minutes at 210° C.

PPP011 Liquid Formulations Used for Vapor Analysis Studies

All PPP011 liquid formulations were prepared at Quantum Pharma and shipped to RPC for vapor analysis studies. Throughout the course of these studies, formulations at 7 different THC and CBD concentrations were prepared. The formulations are listed in order of highest to lowest concentration in Table 46. For initial feasibility testing of the drug, two formulations were prepared, one at a very high (CP33) and one at a very low (CP32) concentration. These formulations were not included as part of the data set used to calculate the dose of PPP011.

In Table 46, “target concentration” represents the THC and CBD concentrations that Quantum Pharma targeted when preparing the formulation. After each formulation arrived at RPC, the THC and CBD concentrations were measured by HPLC. The % error for each formulation (the difference between the target and the measured concentration) is shown. For all calculations, the measured concentrations were used. In every case, 100 μl of formulation was loaded into the drip dosing capsule. As an example, for CP39, the loading dose was 16.10 mg THC and 4.55 mg CBD.

TABLE 46
Calculated and Measured THC and CBD Concentrations
in PPP011 Liquid Formulations

| | | | Measured | | | |
|---|---|---|---|---|---|---|
| | Target | | Concentration | | % Error in | |
| | Concentration | | (by HPLC) | | Target vs | |
| | THC | CBD | THC | CBD | Measured* | |
| Formulation | (mg/ml) | (mg/ml) | (mg/ml) | (mg/ml) | THC | CBD |
| CP33 | 532.0 | 140.0 | 511.3 | 138.8 | 3.9 | 0.9 |
| CP38 | 210.0 | 50.0 | 196.0 | 49.0 | 6.7 | 2.0 |
| CP39 | 175.0 | 50.0 | 161.0 | 45.5 | 8.0 | 9.0 |
| CP35 | 168.5 | 58.2 | 165.0 | 57.5 | 2.1 | 1.2 |
| CP36 | 139.7 | 48.2 | 137.0 | 48.0 | 1.9 | 0.5 |
| CP37 | 112.3 | 38.8 | 112.0 | 38.5 | 0.3 | 0.8 |
| CP32 | 66.5 | 17.5 | 65.2 | 17.5 | 2.0 | 0.0 |

*The measured concentration was always the same or lower than the target concentration.

All the formulations that were included in the dose calculations for PPP011 were prepared partially (CP35, CP36, CP37, CP38) or fully (CP39) under nitrogen. For those prepared partially under nitrogen, ethanol was evaporated from the raw material (THC in ethanol) by mixing on a stir plate and streaming nitrogen gas over the surface of the liquid. The beaker of raw material was covered in plastic film with an entry point for nitrogen inflow and an exit point for nitrogen outflow. Crystalline CBD was then added, and the mixture was reconstituted with the appropriate volume of ethanol. The formulation was open to the air during manipulations (e.g., addition of CBD). After preparation, the formulation was transferred to an amber glass bottle that was purged with nitrogen before closing. CP39 was prepared fully under nitrogen inside a glove bag. During evaporation of ethanol from the THC solution, the beaker was left uncovered since it was prepared in an environment with very little oxygen.

CP32 and CP33, which were used for early exploratory research on the PPP011 formulation, were open to the air during the entire preparation period. Ethanol was evaporated from the THC simply by spinning the solution in an open beaker.

To gain insight into the stability of the liquid formulation, several formulations were re-tested by HPLC after they had been stored in the fridge for a range of time periods. As shown in Table 47, the THC and CBD concentrations of all formulations (including CP32 and CP33, which were exposed to the air for many hours during preparation and subsequent experimentation) were only reduced by approximately 0-5%, even after storage for up to 4 months.

TABLE 47
Measured THC and CBD Concentrations (by HPLC) of PPP011
Liquid Formulations Stored for Various Lengths of Time

| | Storage | THC | % THC | CBD | % CBD |
|---|---|---|---|---|---|
| Formulation | Period (days) | (mg/ml) | Remaining | (mg/ml) | Remaining |
| CP33 | 120 | 488 | 95.4 | 132 | 95.1 |
| CP38 | 36 | 192 | 98.0 | 47 | 95.9 |
| CP35 | 62 | 157 | 95.2 | 55 | 95.7 |
| CP32 | 123 | 65.5 | 100.5 | 17.5 | 100.0 |

### Results: Data and Calculations to Determine Dose of PPP011

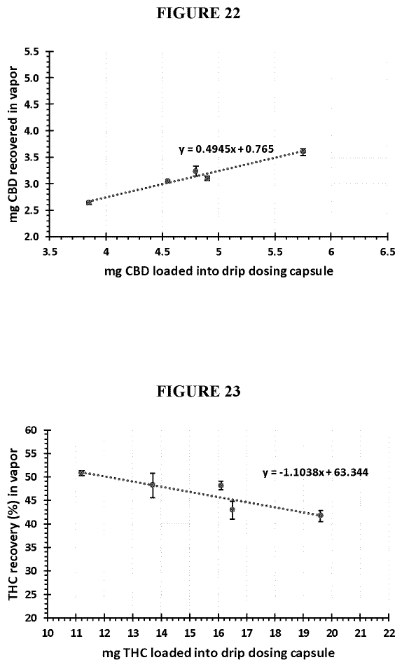

Briefly, the dose of PPP011 was determined in the following way:
Determine the relative recovery of THC and CBD in the vapor of PPP001 (cannabis plant pellet).
Based on relative recovery, calculate the amount of THC and CBD in the vapor of a PPP001 pellet that exactly meets the drug product's specifications (26.6 mg THC and 7 mg CBD) (Table 48).
Determine the relative and absolute recovery of THC and CBD in the vapor of several PPP011 formulations at different concentrations (Table 49).
Construct a curve of “absolute recovery in vapor” vs “loading dose” to determine the dose of PPP011 that results in a vapor match with PPP001 (FIG. 21, FIG. 22, and Table 50).

### Step 1

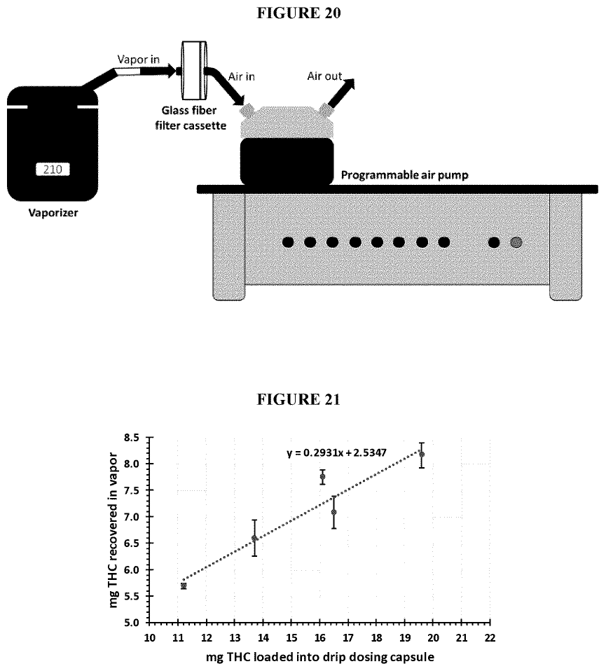

To determine the relative recovery of THC and CBD in the vapor of PPP001 (cannabis plant pellet), 10 pellets from lot 20180910@CP06 were vaporized for 20 minutes at 210° C. and the vapor was collected as shown in FIG. 20. This lot of PPP001 had a THC content of 7.6% and a CBD content of 2.1%. The average pellet weight (±SE) was 290.1 (±2.2) mg. The average relative recovery in the vapor (±SE) was 27.6 (±0.5) % for THC and 45.2 (±0.7) % for CBD.

### Step 2

To determine the amounts of THC and CBD in the vapor of a PPP001 pellet that exactly meets the drug product's specifications, the relative recoveries from Step 1 were used to calculate the absolute recoveries in a pellet with 26.6 mg THC and 7 mg CBD. The results are shown in Table 48. Thus, the goal for PPP011 was to determine the amounts of THC and CBD that needed to be loaded into the drip dosing capsules to produce vapor with 7.3 mg THC and 3.2 mg CBD.

TABLE 48
Target Amount in Vapor Based on PPP001
Target amount in vapor (mg) −/+ 10%

| | Relative | Absolute amount | | |
|---|---|---|---|---|
| Amount of THC | recovery of | of THC in vapor | | |
| in pellet (mg) | THC in vapor (%) | (i.e., target) (mg) | −10% | +10% |
| 26.6 | 27.6 | 7.3 | 6.6 | 8.1 |

Target amount in vapor (mg) −/+ 10%

| | Relative | Absolute amount | | |
|---|---|---|---|---|
| Amount of CBD | recovery of | of CBD in vapor | | |
| in pellet (mg) | CBD in vapor (%) | (i.e., target) (mg) | −10% | +10% |
| 7.0 | 45.2 | 3.2 | 2.8 | 3.5 |

### Step 3

To determine the relationship between THC and CBD loading dose and recovery in the vapor for PPP011, liquid formulations at various concentrations were prepared (see Table 46) and drip dosing capsules were loaded (in a glove bag under nitrogen) with 100 μl of the formulation. The ethanol was evaporated from the formulation under a gentle stream of nitrogen and the drip dosing capsules were vaporized for 6 minutes at 210° C. Table 49 shows the relative and absolute recovery of THC and CBD in the PPP011 vapor.

TABLE 49
Relative and Absolute Recovery in Vapor for PPP011

| | Amount of THC | Sample size | Average relative | Absolute amount |
|---|---|---|---|---|
| | in drip dosing | (# of capsules | recovery of THC in | of THC in |
| Formulation | capsule (mg) | vaporized) | vapor (±SE) (%) | vapor (±SE) (mg) |
| CP38 | 19.6 | 10 | 41.7 (±1.2) | 8.2 (±0.2) |
| CP35 | 16.5 | 10 | 42.9 (±1.8) | 7.1 (±0.3) |
| CP39 | 16.1 | 20 | 48.1 (±0.9) | 7.7 (±0.1) |
| CP36 | 13.7 | 10 | 48.1 (±2.5) | 6.6 (±0.3) |
| CP37 | 11.2 | 5 | 50.8 (±0.5) | 5.7 (±0.1) |
| | Amount of CBD | Sample size | Average relative | Absolute amount |
| | in drip dosing | (# of capsules | recovery of CBD in | of CBD in |
| Formulation | capsule (mg) | vaporized) | vapor (±SE) (%) | vapor (±SE) (mg) |
| CP35 | 5.8 | 10 | 62.5 (±1.2) | 3.6 (±0.1) |
| CP38 | 4.9 | 10 | 63.3 (±0.9) | 3.1 (±0.04) |
| CP36 | 4.8 | 10 | 67.4 (±1.9) | 3.2 (±0.1) |
| CP39 | 4.6 | 20 | 67.0 (±0.7) | 3.0 (±0.03) |
| CP37 | 3.9 | 5 | 68.5 (±0.8) | 2.6 (±0.03) |

### Step 4

To determine the amounts of THC and CBD in PPP011 that would provide a vapor match with PPP001, a curve of “absolute recovery in vapor” vs “loading dose” was constructed for THC (FIG. 21) and CBD (FIG. 22). Using the slope of each line, the dose of PPP011 was calculated (Table 50). Based on these data, the chosen dose of PPP011 was 17 mg THC and 5 mg CBD.

TABLE 50
Calculated Dose of PPP011

| | | Target | | | Amount to load | |
|---|---|---|---|---|---|---|
| | | amount in | | | (mg) to hit | |
| | Target | vapor (mg) | | Amount to load in drip | −/+10% of | |
| | amount in | −/+10% | | dosing capsule (mg) to | vapor target | |
| Cannabinoid | vapor (mg) | −10% | +10% | hit exact vapor target | −10% | +10% |
| THC | 7.3 | 6.6 | 8.1 | 16.4 | 13.9 | 18.9 |
| CBD | 3.2 | 2.8 | 3.5 | 4.9 | 4.2 | 5.5 |

### Physicochemical and Biological Properties

Because the formulation for the finished drug product is so minimal (pure THC and CBD), the physicochemical characteristics of the drug substance also apply here. Two properties of PPP011 that are valuable for its performance are the ability of the drug to vaporize sufficiently (such that a substantial fraction of the loading dose is contained in the vapor), and the ability of the drug to vaporize efficiently at a temperature that the delivery device is capable of reaching.

### Fraction of PPP011 Loading Dose in Vapor

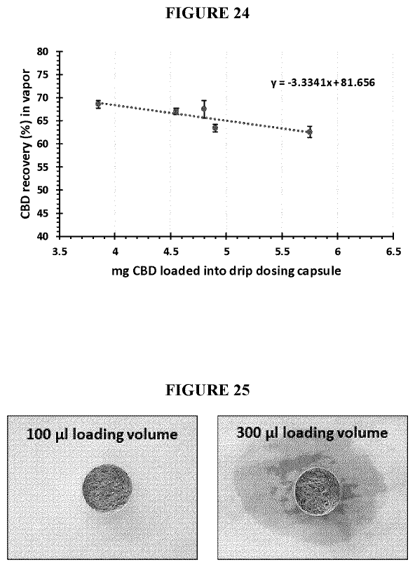

Research was carried out to determine the proportion of THC and CBD that entered the vapor when PPP011 was vaporized using the Mighty Medic®. FIG. 23 and FIG. 24 show a curve of “relative recovery in vapor” vs “loading dose” for THC and CBD, respectively. According to the curve, when a dose of PPP011 (17 mg THC and 5 mg CBD) is vaporized at 210° C., 45% of the THC and 65% of the CBD are present in the vapor. This was a measurement for determining the dose of PPP011 since patients are only exposed to the vaporized fraction of the drug; thus, the dose in the vapor is the true dose that the patient receives.

### Vaporization Temperature of PPP011

A key parameter relevant to the performance of PPP011 is the vaporization temperature of the drug product and the ability of the Mighty Medic® to meet this temperature requirement. Vaporizers are used to heat cannabis or synthetic cannabinoids to a temperature that is high enough to generate cannabinoid vapors (around 180-190° C.), but below the point of combustion where smoke and associated toxins are produced (near 230° C.) (Gieringer et al., 2004). During development of the system for capturing cannabinoids in cannabis vapor, experiments indicated that setting the Mighty Medic® at its maximum temperature (210° C.) resulted in higher relative recoveries in the vapor than setting the device at 195° C. (See Trials 9 and 10 in Example 5). These findings are consistent with two published studies that tested a range of vaporization temperatures using the Volcano@vaporizer (also a Storz & Bickel product), which has a maximum temperature of 230° C. In both studies, THC recovery increased with vaporization temperature, and the highest temperature tested (226° C. in one study and 230° C. in the other) resulted in the greatest recovery (Hazekamp et al., 2006; Pomahacova et al., 2009).

Maximum cannabinoid recovery is not the only consideration when choosing a vaporization temperature. A balance must be achieved between efficient recovery of cannabinoids, user comfort, and abuse potential.

### Manufacturing Process Development

Briefly, PPP011 may be manufactured in the following way:
Prepare a formulation of 170 mg/ml of synthetic THC and 50 mg/ml of synthetic CBD in ethanol by:
   evaporating ethanol from a solution of THC in ethanol;
   adding crystalline CBD;
   and reconstituting with ethanol to achieve final concentrations.
Load 100 μl of the formulation (17 mg THC and 5 mg CBD) into a drip dosing capsule.
Dry the drip dosing capsule to remove the ethanol.
Individually package each drip dosing capsule to keep it protected from oxygen until it is ready to be used.

One of the technically challenging elements of the manufacturing process for PPP011 is performing the entire process under nitrogen. Oxygen degrades cannabinoids (particularly THC); therefore, handling of the raw materials, preparation of the intermediate formulation, loading of the drip dosing capsules, and packaging of the final drug product are performed inside a nitrogen-purged enclosure. Oxygen exposure is avoided during ethanol evaporation from the drug substance (THC in ethanol) and ethanol evaporation from the drip dosing capsules because these procedures are performed under vacuum.

Evaporation of Ethanol from the Drug Substance (THC in Ethanol)

To manufacture PPP011, ethanol may be evaporated from the drug substance (THC in ethanol) so the THC can be more concentrated in the intermediate liquid formulation. A rotary evaporator is used for this step. Rotary evaporation is a standard method for solvent removal due to its speed and thus its ability to remove large volumes of solvent. Earlier batches of PPP011 for R&D work were prepared without a rotary evaporator. Instead, ethanol was evaporated from the drug substance by mixing on a stir plate and streaming nitrogen gas over the surface of the liquid. The beaker of raw material was covered in plastic film with an entry point for nitrogen inflow and an exit point for nitrogen outflow. This method was adequate for the smaller batches that were required for R&D, but it was slow and therefore may not be appropriate for larger batches.

### Mixing of the Intermediate Liquid Formulation

The intermediate formulation may be mixed using a digital benchtop mixer. During dispensing, the formulation may be mixed continuously. This is done to ensure that the formulation remains homogenous throughout the whole dispensing procedure. The intermediate formulations prepared for R&D work were mixed with a magnetic stir bar on a stir plate and were not mixed continuously during dispensing. While a stir bar was adequate for smaller R&D batches, a benchtop mixer may be more effective and more appropriate for mixing larger volumes.

Dispensing of the Intermediate Liquid Formulation into the Drip Dosing Capsules

For speed, accuracy, and reproducibility, a dual syringe continuous liquid dispenser may be used to dispense the PPP011 intermediate liquid formulation into the drip dosing capsules. With the dual syringe design, one syringe fills while the other dispenses, which ensures that the instrument is always ready. This may be ideal for time critical applications that require repetitive dispensing of a single liquid. In contrast, with a single syringe dispenser, the user must wait for the syringe to fill between each round of repeated dispensing. For R&D batches, the formulation was dispensed with a manual aspiration pipette.

### Drying of the Drip Dosing Capsules

A vacuum oven was chosen for drying the drip dosing capsules because it is able to remove the ethanol in a relatively short period of time without heat, it provides protection from oxygen during the drying process because the drug product is under vacuum, and it is able to dry many doses at once. In addition, the oven can be backfilled with nitrogen, so the drug is protected when racks of drip dosing capsules are being loaded and unloaded.

The ability to remove the ethanol without using heat is important for PPP011 because of the sensitivity of cannabinoids to high temperatures. By choosing a vacuum oven as part of the manufacturing process, this provided the option to use very low heat if ethanol evaporation had proven difficult. However, experiments with the vacuum oven and the PPP011 drug product showed that vacuum alone was able to bring the ethanol down to a very low level (0.2 mg per dose).

During R&D work, ethanol was evaporated by leaving the drip dosing capsules inside a nitrogen atmosphere for approximately 90 minutes under a gentle stream of nitrogen. Using this technique, more than 1 mg of ethanol remained on each drip pad. Because the drip dosing capsules were prepared this way for VOC analysis, a large ethanol peak was visible on each PPP011 chromatogram.

### Sealing of the Drip Dosing Capsules

Suitability of the primary and secondary packaging (drip dosing capsules and mylar foil bags) for PPP011.

The mylar foil bags containing the PPP011 drug product are sealed using a precision horizontal impulse heat sealer. The sealer allows the user to specify a sealing pressure, temperature, and time. A digital pressure monitor allows the sealer to cycle only if the sealing pressure is within the range that has been set. For precise temperature control, the heat seal band serves as its own sensing device. The controller instantly responds to the feedback it receives from the heat seal band, thereby keeping the temperature constant. Other primary packaging, secondary packaging, scavengers or sensors as described herein may be used.

For R&D work, the drug product was not sealed since the drip dosing capsules were loaded with formulation, dried, and then used immediately for experiments. For the exploratory stability study, each drip dosing capsule was packaged in a 40 mL precleaned clear glass EPA vial with a 24 mm open-top PTFE lined septa cap.

### Container Closure System

### PPP011 Packaging

For the PPP011 drug product, the drip dosing capsules and the small mylar foil bags may be both considered primary packaging and the large mylar foil bags may be secondary packaging. The primary or secondary packaging may also comprise oxygen or moisture scavengers, indicators of oxygen or moisture content, or both.

The drip dosing capsules were chosen to contain the PPP011 formulation because they are a commercial product available from Storz & Bickel and are designed to function with the Mighty Medic®. When the Mighty Medic® was approved as a medical device, the drip dosing capsules were included in this approval. The drip pad and dosing capsule are made of stainless steel and aluminum, respectively, both of which are ideal because they are low-VOC materials that can tolerate a temperature of 210° C. without the risk of ignition.

Because THC is sensitive to oxygen and light, PPP011 has three packaging criteria: 1) protection from light; 2) sealed in an environment with no or little oxygen; and 3) each dose is sealed individually so that it is not exposed to oxygen until the patient is ready to use it. A simple but effective way to meet these needs is to package each drip dosing capsule in its own small mylar foil bag and seal under nitrogen. As an added layer of protection, groups of 24 small mylar foil bags (a one-week supply of the drug) are then placed inside a large mylar foil bag, which is also sealed under nitrogen. This double layer of protection will remain intact during storage and shipping. Both the large and small mylar foil bags are 4.3 mils thick and have an extremely low oxygen transmission rate of 0.001/cc/m2/24 hours. Other primary packaging, secondary packaging, scavengers or sensors as described herein may be used.

Mylar is a polyester film known for its high tensile strength and barrier properties. The bags used for packaging PPP011 are made from polyester film that has been metallized by applying a uniform layer of aluminium. The layer of metal provides a barrier against light and is also important for preventing THC absorption. THC is known to diffuse into plastics (Garrett and Hunt, 1974; Christophersen, 1986) and so should not be stored in plastic packaging.

### PPP011 Dosing Device

Under laboratory testing conditions, the device used to deliver PPP011 (the Mighty Medic®) delivers an accurate and reproducible dose of the drug. This is shown in Table 49, FIG. 21, and FIG. 22. When the loading dose is varied, the dose in the vapor varies accordingly. When the same dose is loaded into several drip dosing capsules, the amount of THC and CBD in the vapor is consistent between samples (standard error ranges from 0.03 to 0.3 mg). Experiments in the laboratory use a standardized and reproducible method for drawing vapor from the Mighty Medic® (i.e., a smoking machine). This machine can be set to draw vapor continuously (at a specified flow rate) or intermittently (with a set puff duration, puff volume, and puff frequency). Although subjects in a trial can be trained to inhale the drug with a specific puff duration and puff frequency, their puff volume is dependent on their inhalation capacity. Thus, the dose of PPP011 they receive will be affected by their ability to inhale the drug.

### Microbiological Attributes

Microbial limits testing will be performed on all lots of PPP011. The dosage form is relatively dry (THC resin and CBD powder inside a stainless steel and aluminum drip dosing capsule) and the raw materials are synthetic (rather than plant-derived); therefore, there is no reason to expect microbial growth. However, the drug does not possess any growth inhibitory properties, and as such, the following tests will be performed: total aerobic bacteria count, total combined yeasts and molds count, bile tolerant gram-negative bacteria, Escherichia coli, Salmonella species, Staphylococcus aureus, and Pseudomonas aeruginosa.

### Compatibility

General Compatibility of PPP011 with the Mighty Medic®

To deliver PPP011 to patients, testing using an existing commercial system for vaporizing liquid cannabinoid preparations was conducted using the Storz & Bickel Mighty Medic® vaporizer and drip dosing capsules. Thus, the functionality of the system was proven before research was undertaken to use it for PPP011. Patients are already using this system to vape a solution of dronabinol (synthetic THC) dissolved in alcohol. Dronabinol in pure alcohol is not available commercially, but pharmacies will prepare it this way for patients (according to a physician's prescription). The operating manuals for the Volcano Medic® and the Volcano Medic 2@include instructions for vaping dronabinol in alcohol (Storz & Bickel, 2017, 2019b).

The Volcano® is recommended by the manufacturer for vaping dronabinol in alcohol because it is equipped with an air pump that can be used to blow off the alcohol. The pump pulls ambient air into the device and the air is heated as it flows along the device's heater in a spiral pattern. The hot air exits the top of the device, then passes through the filling chamber where the cannabinoids are contained. Vaporized cannabinoids are collected in a valve balloon attached to the filling chamber. When the balloon is full, the user detaches it and inhales the vapor from the balloon. To remove the alcohol from a drip pad loaded with dronabinol in alcohol, the device is heated to 100° C. and the air pump is turned on to blow hot air through the drip pad; this is done without the balloon attached. A temperature of 100° C. is high enough to quickly evaporate the alcohol, but too low to vaporize the THC. In contrast to the Volcano®, the Mighty® does not have an air pump, but instead relies on the user to draw air through the vaporizer during each inhalation. Thus, the Mighty® does not have the functionality to evaporate alcohol into the air before the user begins inhaling.

The Mighty® is an appropriate vaporizer for PPP011 because the drug does not require the user to drip an alcohol-based cannabinoid solution onto the drip pad and then evaporate the alcohol before inhaling the vapor. Instead, this step is already integrated into the manufacturing process, producing a pre-loaded, pre-evaporated drip dosing capsule that is ready for the patient to use.

The Mighty Medic® is a battery-powered medical vaporizer manufactured in compliance with medical device quality requirements. In Canada, it is certified as a Class 2 medical device (license number 96431). The drip dosing capsules are designed to function with the Mighty Medic®. When the vaporizer was approved as a medical device, the drip dosing capsules were included in this approval.

### Loading Volume for PPP011 Intermediate Liquid Formulation

To ensure that the PPP011 liquid formulation was compatible with the drip dosing capsules, an important parameter to determine was the volume of liquid that could be loaded onto the drip pad without leaking through the holes in the bottom of the dosing capsule (see FIG. 17A). Drip dosing capsules were placed on tissue paper and loaded with varying volumes of denatured ethanol. Leaking was observed at loading volumes of 200 and 300 μL, whereas no leaking was observed at 100 μL (FIG. 25). Subsequently, volumes between 100 and 200 μL were loaded in 25-μL increments. No leaking was observed at 125 μL, whereas leaking was observed at 150 μL. Thus, 100 μL was chosen as a safe loading volume.

### Vaporization Temperature for PPP011

One parameter relevant to the compatibility of PPP011 with the Mighty Medic® is the vaporization temperature of the drug product and the ability of the device to meet this temperature requirement. The maximum temperature on the Mighty Medic® is 210° C. Higher temperatures (up to 230° C.) result in greater cannabinoid recovery in the vapor. However, there are other factors to consider when choosing the vaporization temperature for a drug product.

In a study commissioned by Storz & Bickel (LabAssistent Phytochemical Services, 2010), 210° C. was selected as the optimal temperature for the Volcano Medic® (for both ground cannabis plant material and an isolate of pure THC dissolved in ethanol). Although vaporization temperatures of 2200 and 230° C. resulted in higher cannabinoid recoveries in the vapor, higher temperatures may generate vapor with a harsher taste, leading to irritation of the throat and coughing in some users.

Abuse potential was addressed in this study by attempting to vaporize any cannabinoids remaining on the drip pad into a second valve balloon and measuring the cannabinoid content of the second balloon. In this study, at a vaporization temperature of 210° C., almost 6 mg (60%) of the loaded dose (10 mg) of THC was recovered from the vapor contained within the first valve balloon. The second balloon contained less than 0.5 mg of THC. This implies that if a device was left unattended after the user had finished inhaling their balloon, a person would be unable to vaporize an appreciable amount of THC from the residual material in the filling chamber. Thus, a very low abuse potential can be achieved by using the device at 210° C.—it is not necessary to set the temperature any higher to extract the vast majority of THC from the drip dosing capsule (LabAssistent Phytochemical Services, 2010).

Using the system developed, residual cannabinoids were measured on the drip dosing capsule after vaporizing PPP011 at 210° C. for 6 minutes with a pump flow rate of 0.75 L/min. The final dose of PPP011 had not been chosen yet when this experiment was conducted, so approximately 13 mg THC and 3.5 mg CBD were loaded onto the drip pad. Several milligrams (1.5-3.5 mg) remained on the capsules after vaporization, but of the cannabinoids measured, all were undetectable. Thus, the residual mass is not due to THC or CBD. It is hypothesized that this residual material is composed of THC and CBD degradation products; upcoming studies aim to characterize these degradants.

### Vaporization Time for PPP011

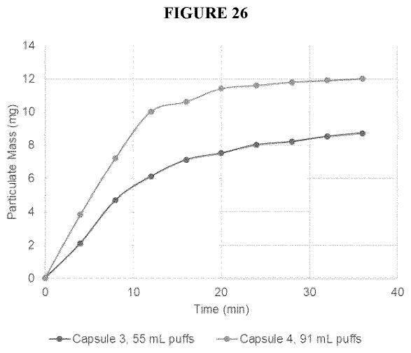

For experiments to determine the dose of PPP011, data were collected by setting the pump flow rate on the smoking machine to 0.75 L/min (i.e., continuous flow). At this rate, approximately 95% of particulate mass collected over 14 minutes was collected within the first 6 minutes. To better understand how long it might take a person to exhaustively vape a PPP011 drip dosing capsule, experiments were performed with the smoking machine using two different puffing topographies. The first topography, published by CORESTA (Cooperation Centre for Scientific Research Relative to Tobacco), consists of a puff duration of 3 seconds, a puff volume of 55 mL, and a puff frequency of 2 puffs/min (CORESTA, 2015). The second smoking topography, published in an academic paper and designed to mirror typical puffing topographies of e-cigarette users, consists of a puff duration of 4 seconds, a puff volume of 91 mL, and a puff frequency of 2 puffs/min (Ogunwale, Chen, et al., 2017). With the smoking machine set to either the 55 mL or 91 mL topography, PPP011 dosing capsules were heated at 210° C. in the Mighty Medic® and particulate mass collected on the filter cassette was recorded every 4 minutes for 36 minutes. Results are shown in FIG. 26.

For both puffing topographies, the curve of particulate mass over time increased sharply until the 12-minute mark, and then began to level off. For the 55 mL topography, 70% of the total particulate mass collected over 36 minutes was collected by 12 minutes. For the 91 mL topography, 83% was collected by this time-point. By 20 minutes, 86% of total particulate mass was collected for the 55 mL topography, and 95% for the 91 mL topography.

Drug delivery may be dependent on puffing topography. The PPP011 clinical protocol specifies the puff duration (3 seconds) and frequency (2 puffs/min), but it is not possible to control a patient's puff volume. In one study that quantified the variation in puffing behaviours among electronic cigarette users, significant variation was observed for all parameters, including mean puff volume, which ranged from 29 mL in one subject to 388 mL in another (Robinson et al., 2015). This same observation was made in previous clinical trials conducted by Tetra Bio-Pharma (PPP001-Ph1-02 and PPP001-Ph1-03): pharmacokinetic parameters were variable between subjects, which was attributed to differences in the subjects' abilities to inhale the drug.

A vaping time of 20 minutes may be sufficient for most patients to consume the majority of the drug. Patients with a larger puff volume will be able to consume almost the whole dose by approximately 15 minutes, but patients with a smaller puff volume may require twice that amount of time. Thus, the PPP011 clinical protocol specifies a vaping time ranging from 15-30 minutes. In a video that will be shown to all trial participants, which explains how to inhale the drug, participants will be told that 20 minutes should be enough time for most of them to consume the full dose.

All citations are hereby incorporated by reference.

The present invention has been described with regard to one or more embodiments. However, it will be apparent to persons skilled in the art that a number of variations and modifications can be made without departing from the scope of the invention as defined in the claims.

### REFERENCES

K. O. Fehr, H. Kalant, Analysis of Cannabis Smoke Obtained Under Different Combustion Conditions, Can. J. Physiol. Pharmacol. 50 (1972) 761-767. doi:10.1139/y72-111.
J. Meehan-Atrash, W. Luo, R. M. Strongin, Toxicant Formation in Dabbing: The Terpene Story, ACS Omega. 2 (2017) 6112-6117. doi:10.1021/acsomega.7b01130.
B. Pomahacova, F. Van der Kooy, R. Verpoorte, Cannabis smoke condensate III: The cannabinoid content of vaporised Cannabis sativa, Inhal. Toxicol. 21 (2009) 1108-1112. doi:10.3109/08958370902748559.
A. Hazekamp, K. Tejkalovi, S. Papadimitriou, Cannabis: From Cultivar to Chemovar II A Metabolomics Approach to Cannabis Classification, Cannabis Cannabinoid Res. 1 (2016) 202-215. doi:10.1089/can.2016.0017.
R. N. Smith, C. G. Vaughan, The decomposition of acidic and neutral cannabinoids in organic solvents, J. Pharm. Pharmacol. 29 (1977) 286-290. https://onlinelibrary.wiley.com/doi/pdf/10.1111/j.2042-7158.1977.tb11313.x.
United Nations Office on Drugs and Crime, Recommended methods for the identification and analysis of cannabis and cannabis products, New York, 2009. doi:ISBN 978-92-1-148242-3.
Aquino, S. (2011). “Gamma radiation against toxigenic fungi in food, medicinal and aromatic herbs.” In A. M6ndez-Vilas (Ed.), Science Against Microbial Pathogens: Communicating Current Research and Technological Advances (pp. 272-281). Formatex Research Center.
Bessonneau, V, & Thomas, O. (2012). “Assessment of exposure to alcohol vapor from alcohol-based hand rubs.” International Journal of Environmental Research and Public Health, 9(3), 868-879.
Boyce, J M, & Pittet, D. (2002). “Guideline for Hand Hygiene in Health-Care Settings: Recommendations of the Healthcare Infection Control Practices Advisory Committee and the HICPAC/SHEA/APIC/IDSA Hand Hygiene Task Force.” Morbidity and Mortality Weekly Report, 51(RR-16), 1-48.
Christophersen, A S. (1986). “Tetrahydrocannabinol stability in whole blood: Plastic versus glass containers.” Journal of Analytical Toxicology, 10, 129-131.
CORESTA. (2015). CORESTA Recommended Method N° 81: Routine Analytical Machine for E-Cigarette Aerosol Generation and Collection—Definitions and Standard Conditions. Paris, France.
Di Stefano, V, Pitonzo, R, Cicero, N, & D'Oca, M C. (2014). “Mycotoxin contamination of animal feedingstuff: detoxification by gamma-irradiation and reduction of aflatoxins and ochratoxin A concentrations.” Food Additives and Contaminants—Part A Chemistry, Analysis, Control, Exposure and Risk Assessment, 31(12), 2034-2039.
Doyle, MP, Applebaum, R S, Brackett, RE, & Marth, EH. (1982). “Physical, chemical and biological degradation of mycotoxins in foods and agricultural commodities.” Journal of Food Protection, 45(10), 964-971.
Fairbairn, J W, Liebmann, JA, & Rowan, MG. (1976). “The stability of cannabis and its preparations on storage.” Journal of Pharmacy and Pharmacology, 28(1), 1-7.
Fenske, JD, & Paulson, SE. (1999). “Human breath emissions of VOCs.” Journal of the Air and Waste Management Association, 49(5), 594-598.
Garrett, ER, & Hunt, CA. (1974). “Physicochemical properties, solubility, and protein binding of delta-9-tetrahydrocannabinol.” Journal of Pharmaceutical Sciences, 63(7), 1056-1064.
Gieringer, D, St. Laurent, J, & Goodrich, S. (2004). “Cannabis Vaporizer Combines Efficient Delivery of THC with Effective Suppression of Pyrolytic Compounds.” Journal of Cannabis Therapeutics, 4(1), 7-27.
Gieringer, DH. (2001). “Cannabis ‘vaporization’: a promising strategy for smoke harm reduction.” Journal of Cannabis Therapeutics, 1(3-4), 153-170.
Gondouin, A, Manzoni, P, Ranfaing, E, Brun, J, Cadranel, J, Sadoun, D, . . . Dalphin, UC. (1996). “Exogenous lipid pneumonia: A retrospective multicentre study of 44 cases in France.” European Respiratory Journal, 9(7), 1463-1469.
Grotenhermen, F. (2003). “Pharmacokinetics and pharmacodynamics of cannabinoids.” Clinical Pharmacokinetics, 42(4), 327-60.
Hadda, V, & Khilnani, GC. (2010). “Lipoid pneumonia: An overview.” Expert Review of Respiratory Medicine, 4(6), 799-807.
Hazekamp, A. (2016). “Evaluating the effects of gamma-irradiation for decontamination of medicinal cannabis.” Frontiers in Pharmacology, 7(108), 1-12.
Hazekamp, A, Ruhaak, R, Zuurman, L, van Gerven, J, & Verpoorte, R. (2006). “Evaluation of a vaporizing device (Volcano) for the pulmonary administration of tetrahydrocannabinol.” Journal of Pharmaceutical Sciences, 95(6), 1308-17.
Kosmider, L, Sobczak, A, Fik, M, Knysak, J, Zaciera, M, Kurek, J, & Goniewicz, ML. (2014). “Carbonyl compounds in electronic cigarette vapors: effects of nicotine solvent and battery output voltage.” Nicotine & Tobacco Research: Official Journal of the Society for Research on Nicotine and Tobacco, 16(10), 1319-26.
LabAssistent Phytochemical Services. (2010). The Volcano® Medic cannabis vaporizer: Optimal temperature for single-dose administration of 100 mg cannabis or 10 mg Dronabinol. Leiden, The Netherlands.
Lucas, PG. (2008). “Regulating compassion: An overview of Canada's federal medical cannabis policy and practice.” Harm Reduction Journal, 5(5), 1-13.
Mechoulam, R. (1970). “Marihuana chemistry.” Science, 168(3936), 1159-1165.
NTP. (1996). Toxicology and Carcinogenesis Studies of 1-Trans-Delta9-Tetrahydrocannabinol (CAS No. 1972-08-3) in F344/N Rats and B6C3F1 Mice (Gavage Studies).
Ogunwale, MA, Chen, Y, Theis, WS, Nantz, MH, Conklin, DJ, & Fu, X-A. (2017). “A novel method of nicotine quantification in electronic cigarette liquids and aerosols.” Analytical Methods, 9(29), 4261-4266.
Ogunwale, MA, Li, M, Ramakrishnam Raju, M V., Chen, Y, Nantz, MH, Conklin, DJ, & Fu, X-A. (2017). “Aldehyde detection in electronic cigarette aerosols.” ACS Omega, 2(3), 1207-1214.
Osborne, A L, Solowij, N, Babic, I, Huang, X-F, & Weston-Green, K. (2017). “Improved Social Interaction, Recognition and Working Memory with Cannabidiol Treatment in a Prenatal Infection (poly I:C) Rat Model.” Neuropsychopharmacology, 42(7), 1447-1457.
Pardridge, WM. (2012). “Drug transport across the blood-brain barrier.” Journal of Cerebral Blood Flow and Metabolism, 32(11), 1959-1972.
Pomahacova, B, Van der Kooy, F, & Verpoorte, R. (2009). “Cannabis smoke condensate III: The cannabinoid content of vaporised Cannabis sativa.” Inhalation Toxicology, 21(13), 1108-1112.
Robinson, RJ, Hensel, EC, Morabito, PN, & Roundtree, KA. (2015). “Electronic cigarette topography in the natural environment.” PLoS ONE, 10(6), 1-14.
Sleiman, M, Logue, J M, Montesinos, VN, Russell, ML, Litter, MI, Gundel, LA, & Destaillats, H. (2016). “Emissions from electronic cigarettes: Key parameters affecting the release of harmful chemicals.” Environmental Science & Technology, 50(17), 9644-51.
Solowij, N. (2018). “Peering Through the Haze of Smoked vs Vaporized Cannabis-To Vape or Not to Vape?” JAMA Network Open, 1(7), e184838.
Solowij, N, Broyd, SJ, van Hell, HH, & Hazekamp, A. (2014). “A protocol for the delivery of cannabidiol (CBD) and combined CBD and A9-tetrahydrocannabinol (THC) by vaporisation.” BMC Pharmacology and Toxicology, 15(1), 58.
Stone, T, Henkle, J, & Prakash, V. (2019). “Pulmonary mucormycosis associated with medical marijuana use.” Respiratory Medicine Case Reports, 26, 176-179.
Storz & Bickel. (2017). Volcano Medic: Instructions for Use. Tuttlingen, Germany.
Storz & Bickel. (2019a). “Storz and Bickel GmbH and Co. KG—Timeline.” https://www.storz-bickel.com/eu/en/sb/.
Storz & Bickel. (2019b). Volcano Medc 2: Instructions for Use. Tuttlingen, Germany.
Tetra Bio-Pharma. (2019). “Tetra Bio-Pharma Redefines Quality Standards for Pharmaceutical Grade Cannabis-Derived Products.” https://ir.tetrabiopharma.com/newsroom/press-releases/news-details/2019/Tetra-Bio-Pharma-Redefines-Quality-Standards-for-Pharmaceutical-Grade-Cannabis-Derived.
Troutt, WD, & DiDonato, MD. (2017). “Carbonyl compounds produced by vaporizing cannabis oil thinning agents.” Journal of Alternative and Complementary Medicine, 23(11), 879-884.
United Nations Office on Drugs and Crime. (2009). Recommended methods for the identification and analysis of cannabis and cannabis products: Manual for use by national drug analysis laboratories. New York: United Nations.
Vethanayagam, D, Pugsley, S, Dunn, EJ, Russell, D, Kay, J M, & Allen, C. (2000). “Exogenous lipid pneumonia related to smoking weed oil following cadaveric renal transplantation.” Canadian Respiratory Journal, 7(4), 338-42.

1.-80. (canceled)

81. A pharmaceutical composition for inhalation by a subject, the pharmaceutical composition comprising: tetrahydrocannabinol (THC); tetrahydrocannabinolic acid (THCA);
cannabidiol (CBD); cannabidiolic acid (CBDA); and cannabigerol (CBG).

82. The pharmaceutical composition of claim 81, in which:
the THC and THCA are present in the pharmaceutical composition at a combined concentration of 1-30%;
the CBD and CBDA are present in the pharmaceutical composition at a combined concentration of 1-20% w/w; and/or
the CBG is present in the pharmaceutical composition at a concentration of 0.1-2% w/w.

83. The pharmaceutical composition of claim 81, wherein the pharmaceutical composition consists of compressed Cannabis sativa, and the Cannabis sativa comprises: THC and THCA at a combined content of 8-11% w/w; CBD and CBDA at a combined content of 2-3% w/w; and CBG at a content of 0.05-0.48% w/w.

84. The pharmaceutical composition of claim 83, wherein the Cannabis Sativa comprises Wilbur, Tamaracouta, and Great Bear cultivars.

85. The pharmaceutical composition of claim 81, wherein the pharmaceutical composition is in unit dose form.

86. The pharmaceutical composition of claim 85, wherein: combustion of the unit dosage form produces smoke that comprises particulate matter; and heating of the unit dosage form to a temperature of 180° C.-230° C. produces vapor that comprises particulate matter.

87. The pharmaceutical composition of claim 86, wherein the particulate matter comprises reproducible amounts of THC, CBD, and CBG for inhalation, thereby enabling reproducible dosing.

88. A composition for vaporization by a subject, the composition comprising: a non-combustible substrate; and a cannabinoid in a neutral form embedded on the non-combustible substrate.

89. The composition of claim 88, wherein the cannabinoid(s) are: synthetic or botanically-derived THC; synthetic or botanically-derived CBD; or synthetic or botanically-derived THC and synthetic or botanically-derived CBD.

90. The composition of claim 88, wherein the non-combustible substrate is a stainless steel mesh.

91. The composition of claim 88, wherein the composition does not comprise an oil, wax, or carrier.

92. The composition of claim 91, wherein the cannabinoid(s) comprise(s): 0.1-50 mg THC; 0.1-100 mg CBD; or 0.1-50 mg THC and 0.1-100 mg CBD.

93. The composition of claim 92, wherein the cannabinoids comprise 13.9-18.9 mg of THC and 4.2-5.5 mg CBD.

94. The composition of claim 88, wherein the composition is in a unit dose form.

95. The composition of claim 94, wherein:
(i) heating of the unit dosage form to a temperature of 180° C.-230° C. produces vapor that comprises particulate matter; and/or
(ii) the particulate matter comprises reproducible amounts of THC and CBD for inhalation, thereby enabling reproducible dosing.

96. A method of preparation of a unit dosage form medicament comprising a noncombustible substrate and a synthetic or botanically-derived cannabinoid or cannabinoids in a neutral form embedded on the non-combustible substrate, the method comprising:
disbursing a liquid composition onto the non-combustible substrate, the liquid composition comprising the cannabinoid or cannabinoids dissolved in a solvent; and
drying the liquid composition to remove the solvent and embed the cannabinoid or cannabinoids onto the non-combustible substrate; and
packaging the non-combustible substrate in an air-tight container to protect it from oxygen exposure.

97. The method of claim 96, wherein the solvent is ethanol.

98. The method of claim 96, wherein drying comprises drying in a vacuum oven.

99. The method of claim 96, wherein one or more steps of the method are conducted in an oxygen-free environment and/or under nitrogen gas.

100. The method of claim 96, wherein the unit dosage form medicament is packaged in an oxygen-free environment in an air-tight container with an oxygen scavenger.
